(19) United States
(12) Patent Application Publication
(10) Pub. No.: US 2024/0060119 A1
(43) Pub. Date: Feb. 22, 2024

(54) METHODS AND COMPOSITIONS FOR SYNCHRONIZING POLYMERASE ACTIVITY IN SITU

(71) Applicant: 10x Genomics, Inc., Pleasanton, CA (US)

(72) Inventor: Felice Alessio BAVA, Rome (IT)

(21) Appl. No.: 18/361,069

(22) Filed: Jul. 28, 2023

Related U.S. Application Data
(60) Provisional application No. 63/393,713, filed on Jul. 29, 2022.

(51) Int. Cl. C12Q 1/682; C12Q 1/6841

(52) U.S. Cl. CPC C12Q 1/682; C12Q 1/6841

(57) ABSTRACT

The present disclosure in some aspects relates to methods and compositions for improved detection and quantification of one or more analytes present in a biological sample. In some aspects, the methods and compositions provided herein address issues associated with the heterogeneity of rolling circle amplification (RCA) products during in situ analyses. In some aspects, the methods and compositions disclosed herein provide more homogenous RCA products for improved sample imaging.

### CROSS-REFERENCE TO RELATED APPLICATION

This application claims priority to U.S. Provisional Patent Application No. 63/393,713, filed Jul. 29, 2022, entitled “METHODS AND COMPOSITIONS FOR SYNCHRONIZING POLYMERASE ACTIVITY INSITU,” which is herein incorporated by reference in its entirety for all purposes.

### FIELD

The present disclosure generally relates to methods and compositions for in situ detection of a plurality of molecules of one or more analytes in a sample.

### BACKGROUND

Genomic, transcriptomic, and proteomic profiling of cells and tissue samples using microscopic imaging can resolve multiple analytes of interest at the same time, thereby providing valuable information regarding analyte abundance and localization in situ. Thus, these in situ assays are important tools, for example, for understanding the molecular basis of cell identity and developing treatment for diseases. In multiplex assays where multiple signals are detected simultaneously, it is important that as much information as possible is collected. However, due to the heterogeneity of analyte abundance (e.g., gene expression levels) and variations among reactions at different locations of a sample, there can be a wide and heterogeneous size and intensity distribution of signal “spots” in the sample. Large signal spots may overlap with one another and/or mask adjacent smaller signal spots, rendering the smaller spots unresolvable. In addition, some analytes may be associated with bright signal spots (e.g., due to high analyte abundance and/or preferential signal amplification), while other analytes may be associated with signal spots that are too dim to be detected simultaneously with the bright spots. There is a need for new and improved methods for in situ assays. The present disclosure addresses these and other needs.

### SUMMARY

In some aspects, provided herein is a method for analyzing a biological sample, comprising: a) contacting the biological sample with a polymerase under a first temperature, wherein the biological sample comprises a plurality of circular nucleic acid molecules and the polymerase is capable of rolling circle amplification (RCA) of the plurality of circular nucleic acid molecules; b) performing RCA in the biological sample using the polymerase under one or more second temperatures higher than the first temperature; c) lowering the temperature of the biological sample to a third temperature; d) performing RCA of the plurality of circular nucleic acid molecules using the polymerase under one or more fourth temperatures higher than the first temperature and/or the third temperature; and e) detecting signals associated with RCA products of the plurality of circular nucleic acid molecules in the biological sample.

In some embodiments, the polymerase is substantially inactive under the first temperature. In any of the preceding embodiments, on average, the extension of nucleic acid molecules hybridized to the plurality of circular nucleic acid molecules by the polymerase under the first temperature may be no more than 5, no more than 10, no more than 50, or no more than 100 nucleotides per hour. In any of the preceding embodiments, on average, the polymerase may produce no more than 1, no more than 5, no more than 10, or no more than 20 copies of the circular nucleic acid molecules per hour under the first temperature, individually. In any of the preceding embodiments, the percent of nucleic acid molecules hybridized to the plurality of circular nucleic acid molecules that are bound to the polymerase under the first temperature may be no more than 5, no more than 10, no more than 25, no more than 50 percent, or no more than 75 percent of the polymerase binding capacity.

In any of the preceding embodiments, the biological sample and the polymerase can be incubated under the first temperature in the presence or absence of dNTPs and/or derivatives and/or analogs thereof. In any of the preceding embodiments, the biological sample and the polymerase can be incubated under the first temperature in the presence of less than about 10 nM, less than about 50 nM, less than about 100 nM, less than about 200 nM, less than about 500 nM, less than about 1 μM, less than about 5 μM, less than about 50 μM, or less than about 100 μM of dNTPs. In any of the preceding embodiments, the biological sample and the polymerase can be incubated under the first temperature in the presence or absence of a cofactor of the polymerase. In some embodiments, the cofactor is Mg²⁺. In any of the preceding embodiments, the biological sample and the polymerase can be incubated under the first temperature in the presence or absence of a di-cation that is not a cofactor of the polymerase. In some instances, the di-cation is Ca²⁺. In any of the preceding embodiments, the biological sample and the polymerase can be incubated under the first temperature for about 5 minutes, about 10 minutes, about 30 minutes, about 1 hour, about 2 hours, about 6 hours, or longer.

In any of the preceding embodiments, the first temperature can be lower than about 15° C., lower than about 10° C., or lower than about 5° C. In some embodiments, the first temperature is about 4° C.

In any of the preceding embodiments, the polymerase can be more active under the one or more second temperatures than under the first temperature. In any of the preceding embodiments, on average, the extension of nucleic acid molecules hybridized to the plurality of circular nucleic acid molecules by the polymerase under the one or more second temperatures may be more than 100, more than 200, more than 500, more than 1,000, more than 1,500, or more than 2,000 bases per minute.

In any of the preceding embodiments, the biological sample and the polymerase may be incubated under the same second temperature. In some embodiments, the same second temperature is about 37° C. In any of the preceding embodiments, the biological sample and the polymerase may be incubated under one second temperature and under a subsequent second temperature higher or lower than the one second temperature. In some embodiments, the one second temperature is about 37° C. and the subsequent second temperature is about 60° C.

In any of the preceding embodiments, the biological sample and the polymerase may be incubated under the one or more second temperatures in the presence of dNTPs and/or derivatives and/or analogs thereof. In any of the preceding embodiments, the biological sample and the polymerase may be incubated under the one or more second temperatures in the presence of a cofactor of the polymerase. In some embodiments, the cofactor is Mg²⁺. In any of the preceding embodiments, the biological sample and the polymerase may be incubated under the one or more second temperatures in the absence of a di-cation that is not a cofactor of the polymerase. In some embodiments, the di-cation is Ca²⁺.

In any of the preceding embodiments, the RCA under the one or more second temperatures may be performed in the same reaction mixture as that of the contacting step under the first temperature. In any of the preceding embodiments, the RCA under the one or more second temperatures may be performed in a different reaction mixture from that of the contacting step under the first temperature.

In any of the preceding embodiments, the biological sample and the polymerase may be incubated under the one or more second temperatures, independently, for about 20 minutes, about 30 minutes, about 1 hour, about 2 hours, about 3 hours, about 4 hours, about 5 hours, about 6 hours, about 8 hours, about 10 hours, about 12 hours, or longer. In any of the preceding embodiments, the one or more second temperatures may be between about 25° C. and about 60° C. In any of the preceding embodiments, the one or more second temperatures may be, independently, about 15° C., about 20° C., about 25° C., about 30° C., about 35° C., about 40° C., about 45° C., about 50° C., about 55° C., or about 60° C.

In any of the preceding embodiments, the polymerase may be substantially inactive under the third temperature. In any of the preceding embodiments, the third temperature may be the same as the first temperature or no more 5° C. higher or lower than the first temperature. In any of the preceding embodiments, the third temperature may be at least about 10° C., at least about 15° C., at least about 20° C., at least about 25° C., at least about 30° C., at least about 35° C., at least about 40° C., at least about 45° C., at least about 50° C., or at least about 55° C. lower than the one or more second temperatures.

In any of the preceding embodiments, on average, the extension of nucleic acid molecules hybridized to the plurality of circular nucleic acid molecules by the polymerase under the third temperature may be no more than 5, no more than 10, no more than 50, or no more than 100 nucleotides per hour. In any of the preceding embodiments, the biological sample and the polymerase may be incubated under the third temperature in the presence or absence of dNTPs and/or derivatives and/or analogs thereof. In any of the preceding embodiments, the biological sample and the polymerase may be incubated under the third temperature in the presence of less than about 10 nM, less than about 50 nM, less than about 100 nM, less than about 200 nM, less than about 500 nM, less than about 1 μM, less than about 5 μM, less than about 50 μM, or less than about 100 μM of dNTPs. In any of the preceding embodiments, the biological sample and the polymerase may be incubated under the third temperature in the presence or absence of a cofactor of the polymerase. In some embodiments, the cofactor is Mg²⁺. In any of the preceding embodiments, the biological sample and the polymerase may be incubated under the third temperature in the presence or absence of a di-cation that is not a cofactor of the polymerase. In some instances, the di-cation is Ca²⁺.

In any of the preceding embodiments, the biological sample and the polymerase may be incubated under the third temperature for about 5 minutes, about 10 minutes, about 30 minutes, about 1 hour, about 2 hours, about 6 hours, or longer. In any of the preceding embodiments, the biological sample and the polymerase may be incubated under the third temperature in the same reaction mixture as that of the RCA under the one or more second temperatures. In any of the preceding embodiments, the biological sample and the polymerase may be incubated under the third temperature in a different reaction mixture from that of the RCA under the one or more second temperatures.

In any of the preceding embodiments, the third temperature may be lower than about 15° C., lower than about 10° C., or lower than about 5° C. In any of the preceding embodiments, the third temperature can be about 4° C.

In any of the preceding embodiments, the method can further comprise an inactivation step between b) and c) to inactivate and/or degrade the polymerase. In some embodiments, the inactivation step comprises incubating the biological sample at a temperature that inactivates the polymerase. In any of the preceding embodiments, the inactivation step can comprise incubating the biological sample at a temperature of 65° C. for at least about 5 seconds, at least about 30 seconds, at least about 1 minute, at least about 5 minutes, or longer. In any of the preceding embodiments, the inactivation step can comprise incubating the biological sample at a temperature of at least 90° C. for at least about 5 seconds, at least about 30 seconds, at least about 1 minute, at least about 5 minutes, or longer.

In any of the preceding embodiments, the inactivation step can comprise treating the biological sample with a proteinase that degrades the polymerase. In some embodiments, the proteinase is proteinase K. In any of the preceding embodiments, the method can further comprise inactivating the proteinase. In any of the preceding embodiments, inactivating the proteinase can comprise incubating the biological sample at a temperature of at least 90° C. for at least about 5 seconds, at least about 30 seconds, at least about 1 minute, at least about 5 minutes, or longer, wherein inactivating the proteinase comprises treating the biological sample with an agent that inactivates the proteinase. In some embodiments, the agent that inactivates the proteinase is a serine protease inhibitor. In some embodiments, the agent comprises phenylmethylsulfonyl fluoride (PMSF), diisopropyl fluorophosphate (DFP), and/or 4-benzenesulfonyl fluoride hydrochloride (AEBSF).

In any of the preceding embodiments, molecules of the polymerase in the biological sample may be irreversibly inactivated and/or degraded. In any of the preceding embodiments, the method can further comprise contacting the biological sample with additional molecules of the polymerase between the inactivation step and lowering the temperature in step c), during step c), and/or after step c). In any of the preceding embodiments, the biological sample and the additional molecules of the polymerase can be incubated under the third temperature for about 5 minutes, about 10 minutes, about 30 minutes, about 1 hour, about 2 hours, about 6 hours, or longer.

In any of the preceding embodiments, the one or more fourth temperatures may be the same as the one or more second temperatures. In any of the preceding embodiments, the one or more fourth temperatures comprise a first fourth temperature of about 37° C. and a subsequent fourth temperature of about 60° C.

In any of the preceding embodiments, the detecting in d) can comprise detecting signals associated with RCA products at multiple locations in the biological sample.

In some aspects, provided herein is a method for analyzing a biological sample, comprising: a) contacting the biological sample with a polymerase and a temperature-sensitive polymer under a first temperature, wherein: the biological sample comprises a plurality of circular nucleic acid molecules, and the temperature-sensitive polymer inhibits rolling circle amplification (RCA) by the polymerase under the first temperature; b) performing RCA of the plurality of circular nucleic acid molecules using the polymerase under a second temperature, which inactivates and/or degrades the temperature-sensitive polymer; and c) detecting signals associated with RCA products of the plurality of circular nucleic acid molecules in the biological sample. In some embodiments, the first temperature is lower than the second temperature. In some embodiments, the second temperature is at least 30° C., at least 35° C., at least 40° C., at least 45° C., at least 50° C., at least 55° C., or at least 60° C. In some embodiments, the first temperature is higher than the second temperature. In some embodiments, the temperature-sensitive polymer comprises a heparin moiety.

In some aspects, provided herein is a method for analyzing a biological sample, comprising: a) contacting the biological sample with a polymerase and a polymerase inhibitor comprising a heparin moiety under a first temperature, wherein: the biological sample comprises a plurality of circular nucleic acid molecules, and the polymerase inhibitor inhibits rolling circle amplification (RCA) by the polymerase under the first temperature; b) performing RCA of the plurality of circular nucleic acid molecules using the polymerase under a second temperature, wherein the polymerase inhibitor is inactivated and/or degraded by a heparin lyase; and c) detecting signals associated with RCA products of the plurality of circular nucleic acid molecules in the biological sample. In any of the preceding embodiments, the polymerase may be substantially inactive in the presence of the polymerase inhibitor. In any of the preceding embodiments, the first temperature may be lower than the second temperature.

In some aspects, provided herein is a method for analyzing a biological sample, comprising: a) contacting the biological sample with a polymerase under a first temperature, wherein the biological sample comprises a plurality of circular nucleic acid molecules and the polymerase is capable of rolling circle amplification (RCA) of the plurality of circular nucleic acid molecules; b) performing RCA in the biological sample using the polymerase under one or more second temperatures higher than the first temperature; c) contacting the biological sample with a proteinase that degrades the polymerase molecules in the biological sample; d) inactivating the proteinase; e) lowering the temperature of the biological sample to a third temperature; f) contacting the biological sample with additional molecules of the polymerase to perform RCA in the biological sample; g) performing RCA of the plurality of circular nucleic acid molecules using the polymerase under one or more fourth temperatures higher than the first temperature and/or the third temperature; and h) detecting signals associated with RCA products of the plurality of circular nucleic acid molecules in the biological sample. In some embodiments, the first contacting step comprises contacting the biological sample with proteinase K. In some embodiments, the proteinase is inactivated by incubating the biological sample at about 90° C. for about 5 seconds, about 30 seconds, about 1 minute, about 5 minutes, or longer, and/or by treating the biological sample with an agent that inactivates the proteinase. In any of the preceding embodiments, the polymerase molecules in the biological sample may be irreversibly inactivated and/or degraded.

In some aspects, provided herein is a method for analyzing a biological sample, comprising: a) contacting the biological sample with a polymerase and a first amount of dNTPs comprising one or more bases, wherein the biological sample comprises a plurality of circular nucleic acid molecules and the polymerase is capable of rolling circle amplification (RCA) of the plurality of circular nucleic acid molecules; b) performing RCA in the biological sample using the polymerase until the first amount of dNTPs is exhausted; c) contacting the biological sample with a second amount of dNTPs comprising all four of A, T, C, and G bases or variants or analogs thereof, d) performing RCA in the biological sample using the polymerase and the second amount of dNTPs; and e) detecting signals associated with RCA products of the plurality of circular nucleic acid molecules in the biological sample. In any of the preceding embodiments, the first amount of dNTPs may comprise all four of A, T, C, and G bases and/or derivatives and/or analogs thereof. In any of the preceding embodiments, the first amount of dNTPs may not comprise any one, two, or three of A, T, C, and G bases and/or derivatives and/or analogs thereof. In any of the preceding embodiments, the first amount of dNTPs can be less than about 10 nM, less than about 50 nM, less than about 100 nM, less than about 200 nM, less than about 500 nM, less than about 1 μM, less than about 5 μM, less than about 50 μM, or less than about 100 μM of dNTPs. In any of the preceding embodiments, the first amount of dNTPs may be exhausted in less than about 1 minute, less than about 5 minutes, less than about 10 minutes, less than about 15 minutes, less than about 20 minutes, less than about 25 minutes, or less than about 30 minutes from the initiation of RCA. In any of the preceding embodiments, the second amount of dNTPs may be more than about 100 μM, more than about 150 μM, more than about 200 μM, more than about 250 μM, or more than about 300 μM, more than about 400 μM, more than about 500 μM, more than about 750 μM, or more than about 1000 μM. In any of the preceding embodiments, the RCA in d) may be performed for more than about 30 minutes, more than about 1 hour, more than about 1.5 hours, more than about 2 hours, more than about 2.5 hours, more than about 3 hours, or more than about 6 hours, without exhausting the second amount of dNTPs.

In any of the preceding embodiments, the polymerase can be selected from the group consisting of Phi29 DNA polymerase, Phi29-like DNA polymerase, M2 DNA polymerase, B103 DNA polymerase, GA-1 DNA polymerase, phi-PRD1 polymerase, Vent DNA polymerase, Deep Vent DNA polymerase, Vent (exo-) DNA polymerase, KlenTaq DNA polymerase, DNA polymerase I, Klenow fragment of DNA polymerase I, DNA polymerase III, T3 DNA polymerase, T4 DNA polymerase, T5 DNA polymerase, T7 DNA polymerase, Bst polymerase, rBST DNA polymerase, N29 DNA polymerase, TopoTaq DNA polymerase, T7 RNA polymerase, SP6 RNA polymerase, T3 RNA polymerase, and variants or derivatives thereof. In any of the preceding embodiments, the polymerase can be a Phi29 polymerase.

### BRIEF DESCRIPTION OF THE DRAWINGS

The drawings illustrate certain embodiments of the features and advantages of this disclosure. These embodiments are not intended to limit the scope of the appended claims in any manner.

FIG. 1 provides a schematic of an exemplary method comprising performing rolling circle amplification (RCA) of a circular nucleic acid molecule using a polymerase with temperature modulation.

FIG. 2 provides a schematic of an exemplary method comprising performing RCA of a circular nucleic acid molecule with a temperature-sensitive polymerase trap to modulate polymerase activity.

FIG. 3 provides a schematic of an exemplary method comprising performing RCA of a circular nucleic acid molecule with a reversible polymerase trap (e.g., a polymerase inhibitor comprising heparin that is reversible by a heparin lyase).

FIG. 4 provides a schematic of an exemplary method comprising performing RCA of a circular nucleic acid molecule with polymerase digestion by a proteinase to modulate polymerase activity.

FIG. 5 provides a schematic of an exemplary method comprising modulating polymerase activity by modulating the amount of dNTPs.

FIGS. 6A-6B show empirical cumulative distribution function plotted against the size of RCPs produced in an example of RCA performed at oscillating temperatures, as compared to control. FIG. 6A shows a graph of the size of RCPs associated with three target genes. FIG. 6B shows a graph of the size of RCPs associated with one of the three target genes following the stripping of a first set of probes for three target genes and incubation with probes for one of the three target genes.

### DETAILED DESCRIPTION

All publications, comprising patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Overview

Rolling circle amplification (RCA) typically produces rolling circle amplification products (RCP) of variable size, which creates challenges for in situ data analysis. Specifically, a sample containing heterogonous RCPs may suffer from reduced signal, optical crowding, and an overall loss of sensitivity. In some aspects, the RCP heterogeneity results from variable diffusion time of one or more reaction component (e.g., polymerase, cofactor, primer) across a biological sample (e.g., a tissue section). In some aspects, the RCP heterogeneity results from variable local concentration of polymerase and/or reagent in a biological sample. In some aspects, the RCP heterogeneity results from unsynchronized timing of RCA reactions in situ. In some aspects, the RCP heterogeneity results from unsynchronized polymerase activity in situ.

Polymerases (e.g., Phi29 polymerase) have ON and OFF rates, which can be assumed to be stochastic. To counter these stochastic rates of polymerase activity, one or more corrective stimuli can be applied to synchronize polymerase activity by forcing the polymerase into the ON or OFF state. Enhanced control over polymerase activity would result in more uniform waves of polymerization by the polymerase, and subsequently, more uniform RCPs in length and size. In some aspects, provided herein are compositions and methods for synchronizing polymerase activity to reduce RCP variability. In some aspects, provided herein are compositions and methods for synchronizing polymerase ON and/or OFF rates. In some aspects, synchronizing polymerase activity is useful in a complex environment such as in a tissue sample. In some aspects, provided herein are compositions and methods for generating in situ RCPs that are more homogeneous in size and intensity. In some aspects, provided herein are compositions and methods to improve in situ target detection and analysis.

In some embodiments, provided herein are compositions and methods for synchronizing polymerase activity using one or more corrective stimuli. In some embodiments, the stimulus for synchronizing polymerase activity is one or more temperatures, wherein a first temperature reduces polymerase activity and one or more second temperatures increase polymerase activity. In some embodiments, the subsequent second temperature is used to inactivate polymerase and other proteins in a biological sample. In some embodiments, a third temperature is used to reduce polymerase activity and one or more fourth temperatures increase polymerase activity. In some embodiments, a temperature cycle is used to synchronize polymerase activity and to produce uniform waves of polymerization. In some embodiments, provided herein are compositions and methods for synchronizing polymerase activity using a temperature to heat inactivate polymerase, wherein the heat inactivation is irreversible. In some embodiments, the temperature used for heat inactivating polymerase is at least 65° C. In some embodiments, the temperature used for heat inactivating polymerase is 90° C.

In some embodiments, provided herein are compositions and methods for synchronizing polymerase activity using a proteinase to degrade polymerase. In some embodiments, the proteinase used for degrading polymerase is proteinase K. In some embodiments, the proteinase K is inhibited with one or more protein K inhibitors. In some embodiments, the proteinase K is heat inactivated, wherein a temperature used for heat inactivating proteinase K is 90° C. In some embodiments, after proteinase K is degraded, new polymerase is added to restore polymerase activity. In some embodiments, provided herein are compositions and methods for synchronizing polymerase activity using a temperature sensitive polymer to inhibit polymerase activity under the first temperature. In some embodiments, the temperature sensitive polymer is inactivated and/or degraded under a second temperature and polymerase activity is restored. In some embodiments, provided herein are compositions and methods for synchronizing polymerase activity using a polymerase inhibitor under a first temperature, wherein the polymerase inhibitor is inactivated and/or degraded by a heparin lyase under a second temperature. In some embodiment, the polymerase inhibitor comprises a heparin moiety. In some embodiments, provided herein are compositions and methods for synchronizing polymerase activity using a first amount of dNTPs comprising one or more bases, wherein the first amount of dNTPs is limiting for polymerase activity. In some embodiments, the polymerase activity is exhausted in the presence of the first amount of dNTPs. In some embodiments, a second amount of dNTPs comprising all four of A, T, C, and G bases or variants or analogs thereof is provided, wherein the second amount of dNTPs is not limiting for polymerase activity.

In some embodiments, the synchronization of polymerase activity leads to more homogeneously sized RCPs and/or brighter RCP signal spots. In some embodiments, an increase in RCP homogeneity leads to a reduction in amplification time. Overall, the synchronization of polymerase activity can improve RCP detection during in situ analysis of a biological sample.

### II. Synchronized Rolling Circle Amplification Insitu

Synchronization of RCA in situ may provide a number of advantages. For instance, synchronization of polymerase activity (e.g., starting the reaction at the same time) of circularized probes targeting sample analytes at different locations in a tissue section may provide more homogeneously sized RCA products. In some aspects, synchronized RCA reactions may lead to signal spots (for detecting RCA products) of homogeneous signal intensity and/or brightness. In some embodiments, the synchronization leads to fewer dim signal spots and/or more bright signal spots for the RCA products. In some embodiments, when RCA product size becomes more homogeneous, the amplification time can be decreased, which makes smaller RCA products of sufficient brightness to be detected. In some embodiments, as RCA product size and brightness become more homogeneous in the same microscope field of view, an RCA reaction time can be achieved so that there are few large signal spots (e.g., even for highly expressed genes) that would overlap with one another and/or mask adjacent smaller signal spots, thus ameliorating the issues of optical crowding In some embodiments, the RCA reaction time can be achieved such that there are few extremely bright signal spots that would render relatively dim spots to be detected simultaneously with the bright spots. In some embodiments, as the RCP size and brightness become more homogeneous in the same microscope field of view, the resolution of neighbouring spots is improved. In some aspects, the synchronized polymerase activity results in at least 40%, at least 50%, at least 60%, at least 70%, at least 80%, at least 90% or at least 95% of the RCPs having a signal within 50% of the mean size when detected. In some aspects, the synchronized polymerase activity results in at least 40%, at least 50%, at least 60%, at least 70%, at least 80%, at least 90% or at least 95% of the RCPs having a signal within 50% of the mean peak intensity. In some aspects, the synchronized polymerase activity results in at least 40%, at least 50%, at least 60%, at least 70%, at least 80%, at least 90% or at least 95% of the RCPs having a signal within 70% to 90% of the mean size when detected. In some aspects, the synchronized polymerase activity results in at least 40%, at least 50%, at least 60%, at least 70%, at least 80%, at least 90% or at least 95% of the RCPs having a signal within 70% to 90% of the mean peak intensity. In some embodiments, the synchronized polymerase activity results in at least 10% more, at least 20% more, at least 30% more, at least 40% more, or at least 50% more detected signal. In some cases, by synchronizing polymerase activity and the start of RCA in the biological sample, it may be easier to distinguish RCA signals from the background signals (e.g., noise).

In some embodiments, rolling circle amplification products are generated using a polymerase selected from the group consisting of Phi29 DNA polymerase, Phi29-like DNA polymerase, M2 DNA polymerase, B103 DNA polymerase, GA-1 DNA polymerase, phi-PRD1 polymerase, Vent DNA polymerase, Deep Vent DNA polymerase, Vent (exo-) DNA polymerase, KlenTaq DNA polymerase, DNA polymerase I, Klenow fragment of DNA polymerase I, DNA polymerase III, T3 DNA polymerase, T4 DNA polymerase, T5 DNA polymerase, T7 DNA polymerase, Bst polymerase, rBST DNA polymerase, N29 DNA polymerase, TopoTaq DNA polymerase, T7 RNA polymerase, SP6 RNA polymerase, T3 RNA polymerase, and variants or derivatives of any of said polymerases.

The amplification products may be immobilized within the matrix generally at the location of the nucleic acid being amplified, thereby creating a localized colony of amplicons. The amplification products may be immobilized within the matrix by steric factors. The amplification products may also be immobilized within the matrix by covalent or noncovalent bonding. In this manner, the amplification products may be considered to be attached to the matrix. By being immobilized to the matrix, such as by covalent bonding or cross-linking, the size and spatial relationship of the original amplicons is maintained. By being immobilized to the matrix, such as by covalent bonding or cross-linking, the amplification products are resistant to movement or unraveling under mechanical stress.

In some aspects, the amplification products are copolymerized and/or covalently attached to the surrounding matrix thereby preserving their spatial relationship and any information inherent thereto. For example, if the amplification products are those generated from DNA or RNA within a cell embedded in the matrix, the amplification products can also be functionalized to form covalent attachment to the matrix preserving their spatial information within the cell thereby providing a subcellular localization distribution pattern. In some embodiments, the provided methods involve embedding the one or more polynucleotide probe sets and/or the amplification products in the presence of hydrogel subunits to form one or more hydrogel-embedded amplification products. In some embodiments, the hydrogel-tissue chemistry described comprises covalently attaching nucleic acids to in situ synthesized hydrogel for tissue clearing, enzyme diffusion, and multiple-cycle sequencing while an existing hydrogel-tissue chemistry method cannot. In some embodiments, to enable amplification product embedding in the tissue-hydrogel setting, amine-modified nucleotides are comprised in the amplification step (e.g., RCA), functionalized with an acrylamide moiety using acrylic acid N-hydroxysuccinimide esters, and copolymerized with acrylamide monomers to form a hydrogel.

Following amplification, the sequence of the amplicon (e.g., RCA product) or a portion thereof, is determined or otherwise analyzed, for example by using detectably labeled probes and imaging. The sequencing or analysis of the amplification products can comprise sequencing by hybridization, sequencing by ligation, and/or fluorescent in situ sequencing, and/or wherein the in situ hybridization comprises sequential fluorescent in situ hybridization. In some instances, a sequence of the RCA product is detected using, e.g., the secondary and higher order probes and detection oligonucleotides described herein. In some embodiments, the detecting step comprises detecting signals associated with RCA products at multiple locations in the biological sample (e.g., as described in Section IV below).

(i) Temperature Modulation

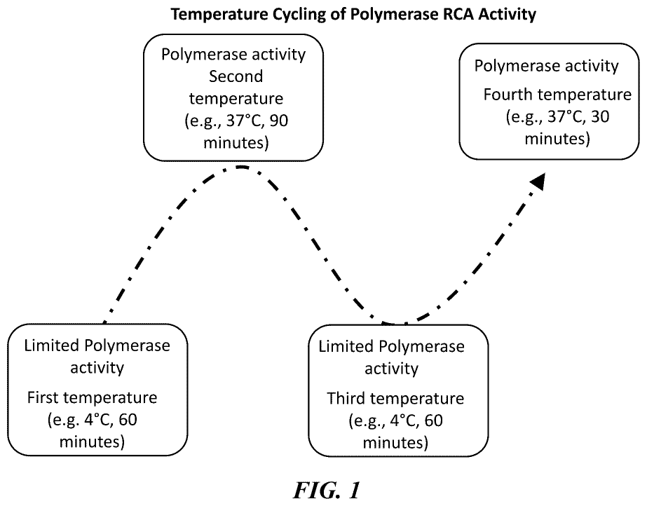

In some embodiments, provided herein is a method for analyzing a biological sample, comprising contacting the biological sample with a polymerase under a first temperature, wherein the biological sample comprises a plurality of circular nucleic acid molecules and the polymerase is capable of rolling circle amplification (RCA) of the plurality of circular nucleic acid molecules. In some embodiments, the method further comprises performing RCA in the biological sample using the polymerase under one or more second temperatures higher than the first temperature (e.g., as shown in FIG. 1). In some embodiments, the method further comprises lowering the temperature of the biological sample to a third temperature. In some embodiments, the method further comprises performing RCA of the plurality of circular nucleic acid molecules using the polymerase under one or more fourth temperatures higher than the first temperature and/or the third temperature. In some embodiments, the method further comprises detecting signals associated with RCA products of the plurality of circular nucleic acid molecules in the biological sample.

In some embodiments, the polymerase is substantially inactive under the first temperature. In some embodiments, the polymerase that is substantially inactive under the first temperature has negligible or significantly reduced polymerase activity. In some embodiments, the polymerase that is substantially inactive under the first temperature remains viable but has negligible or significantly reduced nucleic acid extension activity, reaction product generation, nucleic acid binding, or any combination thereof. In some embodiments, the substantially inactive polymerase under the first temperature has negligible or significantly reduced nucleic acid extension activity. In some embodiments, on average, the extension of nucleic acid molecules (e.g., RCA primers or short RCA products) hybridized to the plurality of circular nucleic acid molecules by the substantially inactive polymerase under the first temperature is no more than 5, no more than 10, no more than 15, no more than 20, no more than 30, no more than 40, no more than 50, no more than 75, or no more than 100 nucleotides per hour. In some embodiments, the substantially inactive polymerase under the first temperature has a negligible or significantly reduced reaction product (e.g., RCP) generation. In some embodiments, on average, the reaction product generated by the polymerase under the first temperature is no more than 1, no more than 2, no more than 3, no more than 4, no more than 5, no more than 10, no more than 15, or no more than 20 copies per hour. In some embodiments, the polymerase under the first temperature has a negligible or significantly reduced nucleic acid binding. In some embodiments, on average, the percent of nucleic acid bound to the polymerase under the first temperature is no more than 5, no more than 10, no more than 15, no more than 20, no more than 25, no more than 30, no more than 40, no more than 50 percent, or no more than 75 percent of the polymerase binding capacity. In some embodiments, the polymerase that is substantially inactive under the first temperature comprises negligible or significantly reduced nucleic acid extension activity, reaction product generation, nucleic acid binding, or a combination thereof. In some aspects, the substantially inactive polymerase under the first temperature has negligible or significantly reduced nucleic acid extension activity of no more than 40%, no more than 30%, no more than 20%, no more than 10%, or no more than 5% of the processivity of the polymerase under a second temperature. In some aspects, the substantially inactive polymerase under the first temperature has negligible or significantly reduced nucleic acid extension activity of no more than 40%, no more than 30%, no more than 20%, no more than 10%, or no more than 5% of the processivity of the polymerase under a reference temperature and condition optimized for polymerase activity (e.g., 30° C.).

In some embodiments, the biological sample and the polymerase are incubated under the first temperature in the presence of deoxynucleoside triphosphates (dNTPs) and/or nucleoside triphosphates (NTPs) and/or derivatives and/or analogs thereof. For instance, the binding mixture can comprise dATP, dTTP, dCTP, and/or dGTP. In some embodiments, the biological sample and the polymerase are incubated under the first temperature in the absence of dNTPs and/or NTPs and/or derivatives and/or analogs thereof. In some embodiments, the biological sample and the polymerase are incubated under the first temperature in the presence of less than about 1 nM, about 10 nM, about 25 nM, about 50 nM, about 100 nM, about 200 nM, about 500 nM, about 1 μM, about 5 μM, less than about 50 μM, or less than about 100 μM of dNTPs.

In some embodiments, the biological sample and the polymerase are incubated under the first temperature in the presence or absence of a cofactor of the polymerase (e.g., a non-protein chemical compound or metallic ion that is required for enzymatic activity). DNA and ribonucleic acid (RNA) polymerases often require a divalent or trivalent metal cofactor cation to catalyze the polymerization of individual nucleotides into a polynucleotide. In some embodiments herein, the presence and/or absence of particular divalent cation(s) can be used to alter the kinetics of polymerase activity. Absent the metal cofactor in the proper oxidation state, polymerization will not occur at an appreciable rate. Metal cations that function as polymerase cofactors include but are not limited to Co²⁺, Mn²⁺, Zn²⁺ and Mg²⁺. Exemplary cofactor cations are disclosed in Vashishtha et al., J Biol Chem 2016; 291(40):20869-20875; US Patent Application Publication No. 2021/0047669; U.S. Pat. Nos. 5,409,811; 8,133,672; 8,658,365; and 9,279,155, all of which are herein incorporated by reference in their entireties. The metal cofactors may be provided in the forms of salts such as MgCl₂ or CoCl₂. The salts form hydrates such as MgCl₂·(H₂O)ₓ or CoCl₂·nH₂O (n=1, 2, 6, and 9) in aqueous solution. One suitable metal cofactor is magnesium. Magnesium may be present as a magnesium salt such as magnesium chloride (MgCl₂). Magnesium may be provided as metallic magnesium, Mg(0), and can be oxidized by electrolysis at an anode in buffered solution to generate Mg(II). Another suitable metal cofactor is cobalt. Cobalt can be provided as a cobalt complex such as a cobalt (III) complex or a cobalt (I) complex. Example cobalt complexes include trans-Dichlorobis(ethylenediamine)cobalt(III) chloride, pentaaminechlorocobalt(III) chloride, hexamine cobalt(III) chloride, trans-dichlorotetrakis(imidazole)cobalt(III) chloride or chlorotris(triphenylphosphine)cobalt(I). The cobalt complex may be reduced or oxidized to cobalt(II) chloride (COCl₂). For example, a Co(III)-complex can be reduced to a Co(II)-complex which can undergo ligand exchange with a buffered aqueous solution to form Co(II) which can then coordinate with a polymerase to activate it for polynucleotide synthesis. A ligand exchange reaction involves the substitution of one or more ligands in a complex ion with one or more different ligands.

Certain divalent or trivalent metal cofactors such as magnesium and manganese h influence the progress of the polymerization reaction. Such catalytic metal cofactors coordinate with a polymerase and the triphosphate of a dNTP to catalyze the addition of a nucleotide to the 3′ terminal nucleotide on the end of the substrate (e.g., a primer). Other metal ions, such as Ca²⁺, can interact with a polymerase, such as Phi29 or a variant or derivative thereof, to negatively impact polymerization (e.g., stabilize the polymerase to slow or halt polymerization). Metal co-factors can have varying catalytic effects upon the polymerization reaction depending upon the various aspects of the polymerization reaction (e.g., polymerase, substrates, reaction conditions, etc.). In some embodiments, the presence and/or absences of polymerase cofactors can be used to synchronize polymerase activity. In some embodiments, the metal cofactors, for example, for Phi29 or a variant or derivative thereof, may include but are not limited to Mg²⁺, Co²⁺, Mn²⁺, or Zn²⁺, or any combination thereof. In some embodiments, the reaction mixture can be substantially free of Mg², Co², Mn²⁺, Zn²⁺, or any combination thereof, so as to halt the polymerase activity while allowing a polymerase (or a polymerase-nucleic acid complex such as a polymerase-primer complex) to diffuse in a sample and bind to circular nucleic acids, primers, and/or complexes thereof.

In some embodiments, the biological sample and the polymerase are incubated under the first temperature in the presence or absence of a cofactor of the polymerase. In some embodiments, the polymerase cofactor is Mg²⁺. In some embodiments, the biological sample and the polymerase are incubated under the first temperature in the presence or absence of a di-cation that is not a cofactor of the polymerase. In some embodiments, the di-cation is Ca²⁺.

In some aspects, the method comprises incubating the biological sample and the polymerase under the first temperature to synchronize polymerase activity. In some embodiments, incubating the biological sample and the polymerase under the first temperature provides time for the polymerase to find a nucleotide-based substrate in the biological sample. In some embodiments, the biological sample and the polymerase are incubated under the first temperature for no more than about 1 minute, no more than about 1 hour, no more than about 2 hours, no more than about 6 hours, no more than about 9 hours, no more than about 12 hours, or no more than about 16 hours. In some embodiments, the biological sample and the polymerase are incubated under the first temperature for about 1 minute, about 5 minutes, about 10 minutes, about 20 minutes, about 30 minutes, about 40 minutes, about 50 minutes, about 1 hour, about 2 hours, about 6 hours, about 9 hours, about 12 hours, or about 16 hours. In some embodiments, the biological sample and the polymerase are incubated under the first temperature for no more than about 16 hours. In some embodiments, incubation at the first temperature for extended periods reduces the RCP homogeneity and the sample signal. In some embodiments, the first temperature used for incubating the biological sample is lower than about 15° C. In some embodiments, the first temperature used for incubating the biological sample is lower than about 15° C., lower than about 10° C., lower than about 9° C., lower than about 8° C., lower than about 7° C., lower than about 6° C., or lower than about 5° C. In some embodiments, the first temperature used for incubating the biological sample is about 4° C.

In some embodiments, the method comprises performing RCA in the biological sample using the polymerase under one or more second temperatures higher than the first temperature. In some embodiments, the polymerase is more active under the one or more second temperatures than under the first temperature. In some embodiments, the polymerase activity comprises nucleic acid extension activity, reaction product (e.g., RCP) generation, nucleic acid binding, and/or any combination thereof. In some embodiments, on average, the extension of nucleic acid molecules hybridized to the plurality of circular nucleic acid molecules by the polymerase under the one or more second temperatures is more than 10, more than 25, more than 50, more than 100, more than 200, more than 500, more than 750, more than 1000, more than 1500, or more than 2000 bases per minute. In some embodiments, the extension of nucleic acid molecules by the polymerase under the one or more second temperatures is approaching the maximal rate of polymerization for the polymerase.

In some embodiments, the second temperature is higher than the first temperature, wherein the polymerase is more active under the one or more second temperatures than under the first temperature. In some embodiments, the second temperature is about 20° C., about 25° C., about 30° C., about 35° C., about 40° C., about 45° C., about 50° C., about 55° C., or about 60° C. In some embodiments, the biological sample and the polymerase are incubated under the same second temperature. In some embodiments, the same second temperature is about 37° C. In some embodiments, the biological sample and the polymerase are incubated under one second temperature and under a subsequent second temperature higher or lower than the one second temperature. In some embodiments, the one second temperature is about 30° C., about 35° C., about 40° C., or about 45° C. and the subsequent second temperature is about 45° C., about 50° C., about 55° C., or about 60° C. In some embodiments, the one second temperature is about 37° C. and the subsequent second temperature is about 60° C.

In some embodiments, the biological sample and the polymerase are incubated under the one or more second temperatures in the presence of dNTPs and/or NTPs and/or derivatives and/or analogs thereof. In some embodiments, the biological sample and the polymerase are incubated under the one or more second temperatures in the presence of a cofactor of the polymerase. In some embodiments, the cofactor of the polymerase is selected from Mg²⁺, Mn²⁺ and combinations thereof. In some embodiments, the biological sample and the polymerase are incubated under the one or more second temperatures in the presence of Mg²⁺. In some embodiments, the biological sample and the polymerase are incubated under the one or more second temperatures in the presence of a non-catalytic metal ion that of the polymerase. In some embodiments, the biological sample and the polymerase are incubated under the one or more second temperatures in the absence of a non-catalytic metal ion that of the polymerase. In some embodiments, the non-catalytic metal is selected from Ca²⁺, Zn²⁺, Co²⁺, Ni²⁺, Eu²⁺, Sr²⁺, Ba²⁺, Fe²⁺, Eu²⁺ and combinations thereof. In some embodiments, the biological sample and the polymerase are incubated under the one or more second temperatures in the absence of Ca²⁺.

In some embodiments, the method comprises performing the RCA under the one or more second temperatures in the same reaction mixture as that of the contacting step under the first temperature. In other embodiments, the method comprises performing the RCA under the one or more second temperatures in a different reaction mixture from that of the contacting step under the first temperature. In some embodiments, the reaction mixture comprises the biological sample, the polymerase, the dNTPs and/or derivatives thereof, and/or cofactors and/or other metals that influence polymerase activity. In some embodiments, the reaction mixture of the contacting step may reduce polymerase activity during the contacting step. In some embodiments, the reaction mixture of the contacting step may help to synchronize polymerase activity before contacting with the different reaction mixture and/or before the performing step. In some embodiments, polymerase activity is regulated by the composition of the reaction mixture (e.g., presence and/or absence of reaction reagents or polymerase cofactors and/or polymerase inhibitors). In some embodiments, the polymerase activity is synchronized in using the same reaction mixture for the contacting step and the performing step, wherein the temperature of the contacting step and the performing step are different. In some embodiments, the polymerase activity is synchronized in using different reaction mixtures for the contacting step and the performing step.

In some embodiments, the biological sample and the polymerase are incubated under the one or more second temperatures, independently, for about 5 minutes, about 10 minutes, about 15 minutes, about 20 minutes, about 30 minutes, about 40 minutes, about 50 minutes, about 1 hour, about 2 hours, about 3 hours, about 4 hours, about 5 hours, about 6 hours, about 8 hours, about 10 hours, about 12 hours, or longer. In some embodiments, the biological sample and the polymerase are incubated under the one or more second temperatures, independently, for different amounts of time, each for about 5 minutes, about 10 minutes, about 15 minutes, about 20 minutes, about 30 minutes, about 40 minutes, about 50 minutes, about 1 hour, about 2 hours, about 3 hours, about 4 hours, about 5 hours, about 6 hours, about 8 hours, about 10 hours, about 12 hours, or longer. In some embodiments, the one or more second temperatures are between about 15° C. and about 60° C. In any of the proceeding embodiments, the one or more second temperatures are, independently, about 15° C., about 20° C., about 25° C., about 30° C., about 35° C., about 40° C., about 45° C., about 50° C., about 55° C., or about 60° C. In any of the proceeding embodiments, the one or more second temperatures are between about 20° C. and about 50° C. In some embodiments, the incubation time can be a range between any of the incubation times listed above (e.g., between about 5 minutes and about 30 minutes, between about 15 minutes and about 2 hours, etc.). In some embodiments, the one or more second temperatures are between any of the temperatures listed above (e.g., between about 20° C. and about 35° C., between about 30° C. and about 50° C., etc.).

In some embodiments, the method comprises lowering the temperature of the biological sample comprising the polymerase to a third temperature. In some embodiments, the polymerase is substantially inactive under the third temperature. In some embodiments, the polymerase that is substantially inactive under the third temperature has negligible or significantly reduced polymerase activity. In some embodiments, the polymerase under the third temperature has negligible or significantly reduced nucleic acid extension activity. In some embodiments, on average, the extension of nucleic acid molecules hybridized to the plurality of circular nucleic acid molecules by the polymerase under the third temperature is no more than 5, no more than 10, no more than 15, no more than 20, no more than 30, no more than 40, no more than 50, no more than 75, or no more than 100 nucleotides per hour. In some embodiments, the polymerase under the first temperature has a negligible or significantly reduced reaction product (e.g., RCP) generation. In some embodiments, on average, the reaction product generated by the polymerase under the first temperature is no more than 1, no more than 2, no more than 3, no more than 4, no more than 5, no more than 10, no more than 15, or no more than 20 copies per hour. In some embodiments, the polymerase under the first temperature has a negligible or significantly reduced nucleic acid binding. In some embodiments, on average, the percent of polymerase in the sample with nucleic acid bound under the first temperature is no more than 5, no more than 10, no more than 15, no more than 20, no more than 25, no more than 30, no more than 40, no more than 50 percent, or no more than 75 percent. In some embodiments, the polymerase that is substantially inactive under the first temperature comprises negligible or significantly reduced nucleic acid extension activity, reaction product generation, nucleic acid binding, or a combination thereof. In some embodiments, the third temperature is the same as the first temperature. In some embodiments, the third temperature is 4° C. In some embodiments, the third temperatures is no more 5° C. higher or lower than the first temperature. In some embodiments, the third temperatures is no more 10° C. higher or lower than the first temperature. In some embodiments, the third temperature is at least about 10° C., at least about 15° C., at least about 20° C., at least about 25° C., at least about 30° C., at least about 35° C., at least about 40° C., at least about 45° C., at least about 50° C., or at least about 55° C. lower than the one or more second temperatures.

In some embodiments, the biological sample and the polymerase are incubated under the third temperature in the presence of dNTPs and/or derivatives and/or analogs thereof. In some embodiments, the biological sample and the polymerase are incubated under the third temperature in the presence of more than about 100 μM, more than about 150 μM, more than about 200 μM, more than about 250 μM, or more than about 300 μM, more than about 400 μM, more than about 500 μM, more than about 750 μM, or more than about 1000 μM of dNTPs. In some embodiments, the biological sample and the polymerase are incubated under the third temperature in the absence of dNTPs and/or derivatives and/or analogs thereof. In some embodiments, the biological sample and the polymerase are incubated under the third temperature in the presence of less than about 1 nM, about 10 nM, about 25 nM, about 50 nM, about 100 nM, about 200 nM, about 500 nM, about 1 μM, about 5 μM, less than about 50 μM, or less than about 100 μM of dNTPs. In some embodiments, the biological sample and the polymerase are incubated under the third temperature in the presence or absence of a cofactor of the polymerase, such as Mg²⁺, Co²⁺, Mn²⁺, or Zn²⁺, or any combination thereof. In some embodiments, the polymerase cofactor is Mg²⁺. In any of the proceeding embodiments, the biological sample and the polymerase are incubated under the third temperature in the presence or absence of a di-cation that is not a cofactor of the polymerase, such as Ca²⁺, Zn²⁺, Co²⁺, Ni²⁺, Eu²⁺, Sr²⁺, Ba²⁺, Fe²⁺, Eu²⁺ and combinations thereof. In some embodiments, the di-cation that is not a cofactor is Ca²⁺.

In some embodiments, the method comprises incubating the biological sample and the polymerase under the third temperature to synchronize polymerase activity for a subsequent round of RCA. In some embodiments, incubating the biological sample and the polymerase under the third temperature provides time for the polymerase to find a nucleotide-based substrate in the biological sample. In some embodiments, the biological sample and the polymerase are incubated under the third temperature for about 1 minute, about 5 minutes, about 10 minutes, about 20 minutes, about 30 minutes, about 40 minutes, about 50 minutes, about 1 hour, about 2 hours, about 6 hours, about 9 hours, about 12 hours, or about 16 hours. In some embodiments, the biological sample and the polymerase are incubated under the third temperature for no more than about 16 hours. In some embodiments, incubation at the third temperature for extended periods reduces the RCP homogeneity and the sample signal. In some embodiments, the biological sample and the polymerase are incubated under the third temperature in the same reaction mixture as that of the RCA under the one or more second temperatures. In other embodiments, the biological sample and the polymerase are incubated under the third temperature in a different reaction mixture from that of the RCA under the one or more second temperatures. In some embodiments, the third temperature used for incubating the biological sample is lower than about 15° C. In some embodiments, the first temperature used for incubating the biological sample is lower than about lower than about 15° C., about 10° C., lower than about 9° C., lower than about 8° C., lower than about 7° C., lower than about 6° C., or lower than about 5° C. In some embodiments, the third temperature is about 4° C.

In some embodiments, the method can further comprise, between the performing RCA under one or more second temperatures and lowering the temperature to a third temperature, an inactivation step to inactivate and/or degrade the polymerase. Polymerase inactivation and/or degradation ensures that polymerase activity is completely stopped before proceeding, wherein polymerase activity can later be restored in a synchronized manner. In some embodiments, the inactivation step comprises incubating the biological sample at a temperature that inactivates the polymerase. In some embodiments, the inactivation step irreversibly inactivates the polymerase. In some embodiments, the inactivation step is performed at a temperature of at least about 65° C. In some embodiments, the inactivation step is performed at a temperature of at least about 65° C., at least about 70° C., at least about 75° C., at least about 80° C., at least about 85° C., at least about 90° C., at least about 95° C., or at least about 100° C. In some embodiments, the inactivation step is performed at a temperature that inactivates the polymerase for about 5 seconds, about 10 seconds, about 20 seconds, about 30 seconds, about 1 minute, about 5 minutes, about 10 minutes, about 20 minutes, about 30 minutes or longer. In some embodiments, the inactivation step is performed at about 65° C. for about 5 seconds, about 10 seconds, about 20 seconds, about 30 seconds, about 1 minute, about 5 minutes, about 10 minutes, about 20 minutes, about 30 minutes or longer. In some embodiments, the inactivation step is performed at about 90° C. for about 5 seconds, about 10 seconds, about 20 seconds, about 30 seconds, about 1 minute, about 5 minutes, about 10 minutes, about 20 minutes, about 30 minutes or longer. In some embodiments, incubation at an inactivating temperature results in a an inactivated polymerase that is unable to perform further RCA polymerization reactions.

In other embodiments, the inactivation step comprises treating the biological sample with a protein cleavage enzyme that degrades the polymerase, such as a proteinase, protease, or peptidase. Proteinases are protein digesting enzymes that cleave peptide bonds in a protein to yield smaller peptide fragments. Proteinases, which are similar in function to proteases and peptidases and are described interchangeable, can be used to non-specifically inactivate proteins in a biological sample via protein digestion. In some embodiments, the inactivation step comprises treating the biological sample with one or more serine proteinase, cysteine protease, threonine protease, aspartic protease, guamic protease, metalloprotease, and/or asparagine peptide lyase. In some embodiments, the inactivation step comprises treating the biological sample with proteinase K. In some embodiments, the method further comprises inactivating the proteinase. In some embodiments, the proteinase is inactivated by incubating the biological sample at about 90° C. for about 5 seconds, about 10 seconds, about 20 seconds, about 30 seconds, about 1 minute, about 5 minutes, about 10 minutes, about 20 minutes, about 30 minutes or longer. In some embodiments, the proteinase is inactivated by treating the biological sample with an agent that inactivates the proteinase. In some embodiments, the proteinase is inactivated by treating the biological sample with an proteinase inhibitor. In some embodiments, the agent that inactivates the proteinase is a serine proteinase inhibitor. In some embodiments, the agent comprises phenylmethylsulfonyl fluoride (PMSF), diisopropyl fluorophosphate (DFP), and/or 4-benzenesulfonyl fluoride hydrochloride (AEBSF). In some embodiments, the proteinase is inactivated by treating the biological sample with one or more serine proteinase inhibitors for about 1 minute, about 5 minutes, about 10 minutes, about 20 minutes, about 30 minutes or longer. In some embodiments, the proteinase is inactivated by incubating the biological sample at a temperature of at least 90° C. (e.g., for at least 1 minute, at least 5 minutes, at least 10 minutes, at least 15 minutes, or at least 20 minutes). In some embodiments, molecules of the polymerase in the biological sample are irreversibly inactivated and/or degraded. In some embodiments, all or nearly all of the molecules of the polymerase in the biological sample are irreversibly inactivated and/or degraded.

A biological sample having been treated to inactivate or degrade all of the molecules of the polymerase can be subsequently contacted with new molecules of polymerase. In doing so, the RCA reaction can be resumed in a deliberate and synchronized manner, thus preserving the homogeneity of RCPs produced from the polymerase-driven RCA reactions. The biological sample can be contacted with additional polymerase at different times and/or different temperatures, wherein the parameters of time and temperatures can influence the extent of polymerase activity in the biological sample. In some embodiments, the method further comprises contacting the biological sample with additional molecules of the polymerase between the inactivation step and lowering the temperature to a third temperature. In some embodiments, the method further comprises contacting the biological sample with additional molecules of the polymerase during lowering the temperature to a third temperature. In some embodiments, the method further comprises contacting the biological sample with additional molecules of the polymerase after lowering the temperature to a third temperature. In some embodiments, the additional molecules of the polymerase are substantially inactive under a third temperature. In some embodiments, the biological sample and the additional molecules of the polymerase are incubated under the third temperature for about 1 minute, about 5 minutes, about 10 minutes, about 20 minutes, about 30 minutes, about 40 minutes, about 50 minutes, about 1 hour, about 2 hours, about 6 hours, or longer. In some embodiments, lowering the temperature of the biological sample before contacting with additional molecules of the polymerase limits uncontrolled polymerase activity in the biological sample. In some embodiments, lowering the temperature of the biological sample before contacting with additional molecules of the polymerase permits the polymerase to complex with the nucleotide-based substrate across the biological sample without permitting polymerase activity. In some embodiments, lowering the temperature of the biological sample before contacting with additional molecules of the polymerase helps to synchronize polymerase activity across the biological sample to improve reaction product (e.g., RCP) homogeneity in terms of size and intensity to improve overall signal output (e.g., for in situ analysis).

In some embodiments, the method further comprises performing RCA in the biological sample using the polymerase under one or more fourth temperatures higher than the first and/or third temperatures. In some embodiments, the polymerase is more active under the one or more fourth temperatures than under the first and/or third temperatures. In some embodiments, the one or more fourth temperatures are the same as the one or more second temperatures. In any of the proceeding embodiments, the one fourth temperature is about 37° C. and the subsequent fourth temperature is about 60° C. In some embodiments, the method comprises temperatures cycling as a means of modulating and synchronizing polymerase activity. In some embodiments, the synchronization of polymerase activity from using different temperatures results in an increase in homogeneity of RCPs. In some embodiments, more homogenous RCPs in terms of RCP size and intensity reduce optical crowding, increase the number of RCP signals observed, and improve overall RCP signal in the biological sample.

(ii) Temperature-Sensitive Polymerase Traps

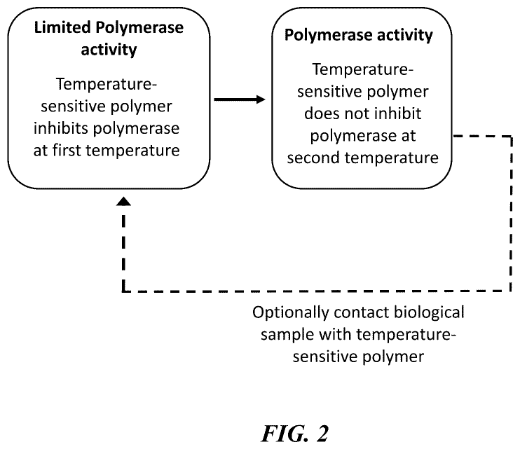

Provided herein are methods for modulating polymerase activity by binding and/or inhibiting a polymerase using a compound or molecule. Compounds containing a heparin-moiety and derivatives thereof are potent inhibitors of DNA polymerase enzymes. In addition to temperature regulation of polymerase activity for the purposes of polymerase activity and RCA reaction synchronization, employment of an tunable inhibition system (e.g., a temperature-sensitive small molecule inhibitor) can influence the rate of polymerase activity. For example, contacting the biological sample comprising the polymerase with a temperature-sensitive trap molecule, wherein the temperature-sensitive trap molecule inhibits polymerase activity under a first temperature and permits polymerase activity under a second temperature, can synchronize polymerase activity. In some embodiments, the polymerase is contacted with a polymerase binder and/or inhibitor (e.g., heparin or a temperature-sensitive polymer such as Hep-PCLA). Similar to the methods described above, changing temperatures can be used indirectly to influence polymerase activity through the temperature-sensitive function of a trap molecule. In some embodiments, provided herein is a method for analyzing a biological sample, comprising contacting the biological sample with a polymerase and a temperature-sensitive polymer under a first temperature, wherein the biological sample comprises a plurality of circular nucleic acid molecules, and the temperature-sensitive polymer inhibits rolling circle amplification (RCA) by the polymerase under the first temperature. In some embodiments, the method further comprises performing RCA of the plurality of circular nucleic acid molecules using the polymerase under a second temperature, which inactivates and/or degrades the temperature-sensitive polymer (e.g., as shown in FIG. 2). In some embodiments, the method further comprises detecting signals associated with RCA products of the plurality of circular nucleic acid molecules in the biological sample.

In some instances, after performing RCA at the second temperature, the method comprises incubating the biological sample at a third temperature, wherein the temperature-sensitive polymer inhibits RCA by the polymerase under the third temperature. In some cases, the third temperature is the same as the first temperature (e.g., as shown in FIG. 2). In some embodiments, the third temperature is different from the first temperature.

In some embodiments, the polymerase is substantially inactive under the first temperature. In some embodiments, the polymerase that is substantially inactive under the first temperature has negligible or significantly reduced polymerase activity. In some embodiments, the polymerase that is substantially inactive under the first temperature comprises negligible or significantly reduced nucleic acid extension activity, reaction product generation, nucleic acid binding, or any combination thereof. In some embodiments, the polymerase is more active under the one or more second temperatures than under the first temperature. In some embodiments, the presence and/or absence of dNTPs and/or NTPs and/or derivatives and/or analogs thereof can influence the rate of polymerase activity. In some embodiments, the presence and/or absence of a cofactor of the polymerase (e.g., Mg²⁺) can influence the rate of polymerase activity. In some embodiments, the presence and/or absence of a metal ion that is not a cofactor of the polymerase (e.g., Ca²⁺) can influence the rate of polymerase activity. In some embodiments, changing one or more components of the reaction mixture can be used in part to synchronize polymerase activity and RCA reactions.

In some embodiments, the method comprises using a first temperature that is lower than a second temperature. In some embodiments, the first temperature used for incubating the biological sample is lower than about 20° C., lower than about 15° C., lower than about 10° C., lower than about 9° C., lower than about 8° C., lower than about 7° C., lower than about 6° C., or lower than about 5° C. In some embodiments, the first temperature used for incubating the biological sample is about 4° C. In some embodiments, the temperature-sensitive polymer substantially inhibits polymerase activity under the first temperature. In some embodiments, the second temperature is at least about 30° C., at least about 35° C., at least about 40° C., at least about 45° C., at least about 50° C., at least about 55° C., or at least about 60° C. In some embodiments, the second temperature is between about 30° C. and about 60° C., between about 30° C. and about 40° C., or between about 35° C. and about 50° C. In some embodiments, the temperature-sensitive polymer is inactivated, disrupted, dissolved, and/or degraded under the second temperature, and the polymerase activity is restored. In some embodiments, the third temperature used for incubating the biological sample is lower than about 20° C., lower than about 15° C., lower than about 10° C., lower than about 9° C., lower than about 8° C., lower than about 7° C., lower than about 6° C., or lower than about 5° C. In some embodiments, the first temperature used for incubating the biological sample is about 4° C. In some embodiments, the temperature-sensitive polymer substantially inhibits polymerase activity under the third temperature.

In other embodiments, the method comprises using a first temperature that is higher than a second temperature. In some embodiments, the first temperature used for incubating the biological sample is higher than about 20° C., higher than about 25° C., higher than about 30° C., higher than about 35° C., or higher than about 37° C. In some embodiments, the first temperature used for incubating the biological sample and temperature-sensitive polymer is about 37° C. In some embodiments, the first temperature used for incubating the biological sample and the temperature-sensitive polymer is between about 30° C. and about 60° C., between about 30° C. and about 40° C., or between about 35° C. and about 50° C. In some embodiments, the temperature-sensitive polymer inhibits polymerase activity under a higher first temperature, wherein the temperature-sensitive polymer polymerizes at higher temperatures. In some embodiments, the temperature-sensitive polymer is inactivated and/or non-functional (e.g., non-inhibitory) under a lower second temperature, and the polymerase activity is restored. In some embodiments, the second temperature is lower than about 30° C., lower than about 25° C., or lower than about 20° C. In some embodiments, the second temperature is between about 18° C. and about 30° C., between about 20° C. and about 25° C., or between about 25° C. and about 30° C. In some embodiments, the biological sample is incubated at a third temperature, wherein the temperature sensitive-polymer inhibits RCA by the polymerase at the third temperature. The third temperature can be the same as the first temperature or different. In some embodiments, the third temperature used for incubating the biological sample is higher than about 20° C., higher than about 25° C., higher than about 30° C., higher than about 35° C., or higher than about 37° C. In some embodiments, the third temperature used for incubating the biological sample and temperature-sensitive polymer is about 37° C. In some embodiments, the third temperature used for incubating the biological sample and the temperature-sensitive polymer is between about 30° C. and about 60° C., between about 30° C. and about 40° C., or between about 35° C. and about 50° C. The biological sample can be cycled between temperatures at which the temperature-sensitive polymer inhibits the polymerase and temperatures at which the temperature sensitive polymer does not inhibit the polymerase any number of times.

Examples of temperature sensitive polymers include but are not limited to heparin-bearing poly(ε-caprolactone-co-lactide)-b-poly(ethylene glycol)-b-poly(ε-caprolactone-co-lactide) (Hep-PCLA). Hep-PCLA conjugates are capable of undergoing temperature-induced solution-to-gel transitions in an aqueous solution. The gelation rate, mechanical strength, and viscosity of Hep-PCLA conjugates are controllably tunable by varying the graft density of PCLA copolymers to heparin. In some embodiments, Hep-PCLA forms a gel at about 37° C., thereby inhibiting polymerase activity. In some embodiments, Hep-PCLA forms a free-flowing aqueous solution at 25° C., thereby allowing polymerase activity.

In some embodiments, the temperature-sensitive polymer is a polymerase inhibitor that can be modulated with temperature. In some embodiments, the temperature-sensitive polymer comprises a heparin moiety. In some embodiments, the temperature-sensitive polymer is heparin and/or a derivative thereof. In some embodiments, the temperature-sensitive polymer comprises a gel-forming molecule. In some embodiments, the limited polymerase activity under the first temperature allows polymerase activity to be synchronized prior to switching to a second temperature that is permissive for polymerase activity. In some embodiments, the use of a temperature-sensitive polymer with different temperatures enables synchronization of the polymerase activity in a biological sample. In some embodiments, the use of a temperature-sensitive polymer with different temperatures results in synchronization of the polymerase activity and more homogenous RCP in terms of size and intensity.

In any of the proceeding embodiments, the detecting step comprises detecting signals associated with RCA products at multiple locations in the biological sample. In any of the proceeding embodiments, the detecting step comprises an in situ analysis for detecting signals associated with RCA products at multiple locations in the biological sample.

(iii) Reversible Polymerase Traps

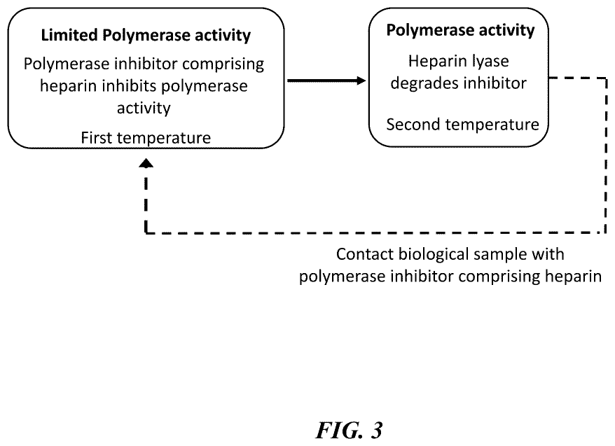

As described above, compounds containing a heparin-moiety and derivatives thereof are potent inhibitors of DNA polymerase enzymes. In addition to temperature regulation and tunable inhibitors, polymerase activity and RCA reaction synchronization can be achieved through the employment of an acute inhibition system (e.g., small molecule inhibitor and an inhibition reversal system). In some aspects, the biological sample comprising the polymerase is contacted with a heparin-moiety or derivatives thereof to remove or trap excess polymerase, thereby reducing non-specific binding in the biological sample. In some embodiments, reversible polymerase trap molecules can be used to synchronize polymerase activity, wherein the biological sample comprising the polymerase is contacted with a trap molecule that inhibits polymerase activity until the biological sample is further contacted with an inhibitor of the trap molecule. In some embodiments, provided herein is a method for analyzing a biological sample, comprising contacting the biological sample with a polymerase and a polymerase inhibitor comprising a heparin moiety under a first temperature, wherein the biological sample comprises a plurality of circular nucleic acid molecules, and the polymerase inhibitor inhibits rolling circle amplification (RCA) by the polymerase under the first temperature. In some embodiments, the method further comprises performing RCA of the plurality of circular nucleic acid molecules using the polymerase under a second temperature, wherein the polymerase inhibitor is inactivated and/or degraded by a heparin lyase (e.g., as shown in FIG. 3). In some embodiments, the method further comprises detecting signals associated with RCA products of the plurality of circular nucleic acid molecules in the biological sample.

Heparin lyases, such as heparinases, are a general class of enzymes that are capable of specifically cleaving the major glycosidic linkages in heparin and heparan sulfate. Three heparinases have been identified in Flavobacterium heparinum, a GAG-utilizing organism that also produces exoglycoronidases, glycosidases, sulfoesterases, and sulfamidases and other enzymes which further act on the lyase-generated oligosaccharide products (Yang, et al. J. Biol. Chem. 260, 1849-1857 (1987); Galliher, et al. Eur. J. Appl. Microbiol. Biotechnol. 15, 252-257 (1982). These lyases are designated as heparinase I (heparinase, EC 4.2.2.7), heparinase II (heparinase II, no EC number) and heparinase III (heparinase EC 4.2.2.8). The three purified heparinases differ in their capacity to cleave heparin and heparan sulfate: Heparinase I primarily cleaves heparin, heparinase III specifically cleaves heparan sulfate; and heparinase II acts on both heparin and heparan sulfate. Several Bacteroides species (Saylers, et al. Appl. Environ. Microbiol. 33, 319-322 (1977); Nakamura, et al. J. Clin. Microbiol. 26, 1070-1071 (1988)) also produce heparin lyases. A heparin lyase has also been purified to apparent homogeneity from an unidentified soil bacterium by Bohmer, et al. J. Biol. Chem. 265, 13609-13617 (1990). Heparin lysases are available for purchase from New England Biolabs, among other chemical and biological commercial entities.

In some embodiments, the polymerase is substantially inactive under the first temperature. In some embodiments, the polymerase that is substantially inactive under the first temperature has negligible or significantly reduced polymerase activity. In some embodiments, the polymerase that is substantially inactive under the first temperature comprises negligible or significantly reduced nucleic acid extension activity, reaction product generation, nucleic acid binding, or any combination thereof. In some embodiments, the polymerase is more active under the second temperatures than under the first temperature. In some embodiments, the presence and/or absence of dNTPs and/or NTPs and/or derivatives and/or analogs thereof can influence the rate of polymerase activity. In some embodiments, the presence and/or absence of a cofactor of the polymerase (e.g., Mg²⁺) can influence the rate of polymerase activity. In some embodiments, the presence and/or absence of a metal ion that is not a cofactor of the polymerase (e.g., Ca²⁺) can influence the rate of polymerase activity. In some embodiments, the presence and/or absence of one or more components of the reaction mixture can influence the rate of polymerase activity. In some embodiments, changing one or more components of the reaction mixture can be used in part to synchronize polymerase activity and RCA reactions.

In some embodiments, the trap molecule is a polymerase inhibitor that can be modulated by its presence and absence in the biological sample. In some embodiments, the trap molecule comprises a heparin moiety. In some embodiments, the trap molecule is heparin and/or a derivative thereof. In some embodiments, the trap molecule comprises a gel-forming molecule. In some embodiments, the limited polymerase activity under the first temperature allows polymerase activity to be synchronized prior to switching to a second temperature that is permissive for polymerase activity. In some embodiments, contacting the biological sample with the trap molecule synchronizes the polymerase activity in the biological sample. In some embodiments, modifying the presence and absence of the trap molecule results in synchronization of the polymerase activity and more homogenous RCP in terms of size and intensity. In some embodiments, the presence and absence of the trap molecule can be modified with an inhibitor of the trap molecule, wherein the inhibitor is a protein, an enzyme, a molecule, a chemical compound, an ion, or any combination thereof. In some embodiments, the presence and absence of the trap molecule can be modified with an inhibitor-specific enzyme. In some embodiments, the presence and absence of the trap molecule can be modified with heparin-lyase. In some embodiments, contacting the biological sample first with the trap molecule and then with an inhibitor of the trap molecule can synchronize the polymerase activity, resulting in more homogenous RCP in terms of size and intensity.

In some embodiments, the polymerase is substantially inactive in the presence of the trap molecule (e.g., polymerase inhibitor). In some embodiments, the polymerase that is substantially inactive in the presence of the trap molecule has negligible or significantly reduced polymerase activity. In some embodiments, the polymerase that is substantially inactive in the presence of the trap molecule comprises negligible or significantly reduced nucleic acid extension activity, reaction product generation, nucleic acid binding, or any combination thereof. In some embodiments, the polymerase is no more active under the second temperatures than under the first temperature in the presence of the trap molecule. In some embodiments, modifying the presence and absence of the trap molecule with an inhibitor of the trap molecule at a first temperature synchronizes polymerase activity. In some embodiments, modifying the presence and absence of the trap molecule with an inhibitor of the trap molecule at a second temperature permits synchronized polymerase activity and RCA reactions.

In some embodiments, the first temperature is lower than the second temperature. In some embodiments, the first temperature used for incubating the biological sample is lower than about 20° C., lower than about 15° C., lower than about 10° C., lower than about 9° C., lower than about 8° C., lower than about 7° C., lower than about 6° C., or lower than about 5° C. In some embodiments, the first temperature used for incubating the biological sample is about 4° C. In some embodiments, the second temperature is about 20° C., about 25° C., about 30° C., about 35° C., about 40° C., about 45° C., about 50° C., about 55° C., or about 60° C. In some embodiments, the trap molecule is unaffected by the first temperature or the second temperature. In some embodiments, the trap molecule is inactivated, disrupted, dissolved, and/or degraded under the second temperature, and the polymerase activity is restored.

In any of the proceeding embodiments, the detecting step comprises detecting signals associated with RCA products at multiple locations in the biological sample. In any of the proceeding embodiments, the detecting step comprises an in situ analysis for detecting signals associated with RCA products at multiple locations in the biological sample.

(iv) Polymerase Digestion

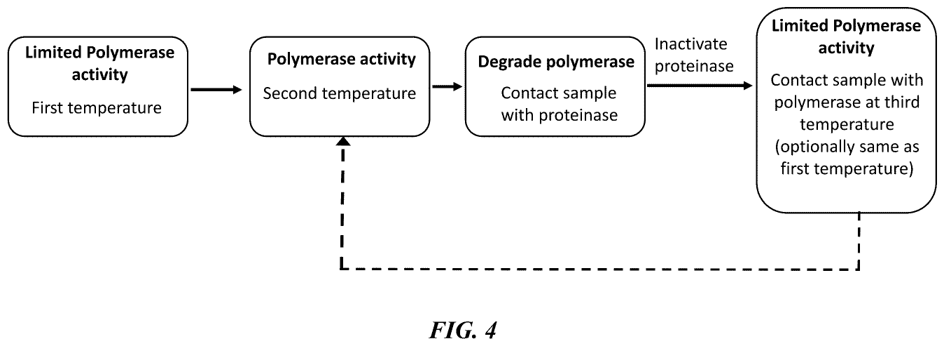

In some aspects, provided herein are methods that improve the control over the polymerase activity by acute termination of polymerase activity is possible by using a protein digestion enzyme (e.g., a proteinase) to the biological sample. For example, contacting the biological sample comprising the polymerase with a proteinase, wherein the proteinase digests the polymerase in the biological sample and inhibits polymerase activity, can further synchronize polymerase activity (e.g., compared to temperature cycling alone). Similarly to the methods described above, changing temperatures can be used to directly influence polymerase activity while addition of a protein digestion enzyme can ensure rapid halting of polymerase activity through polymerase protein digestion. In some embodiments, provided herein is a method for analyzing a biological sample, comprising contacting the biological sample with a polymerase under a first temperature, wherein the biological sample comprises a plurality of circular nucleic acid molecules and the polymerase is capable of rolling circle amplification (RCA) of the plurality of circular nucleic acid molecules. In some embodiments, the method further comprises performing RCA in the biological sample using the polymerase under one or more second temperatures higher than the first temperature. In some embodiments, the method further comprises contacting the biological sample with a proteinase that degrades the polymerase molecules in the biological sample (e.g., as shown in FIG. 4). In some embodiments, the method further comprises inactivating the proteinase. In some embodiments, the method further comprises lowering the temperature of the biological sample to a third temperature. In some embodiments, the method further comprises contacting the biological sample with additional molecules of the polymerase to perform RCA in the biological sample. In some embodiments, the method further comprises performing RCA of the plurality of circular nucleic acid molecules using the polymerase under one or more fourth temperatures higher than the first temperature and/or the third temperature. In some embodiments, the method further comprises detecting signals associated with RCA products of the plurality of circular nucleic acid molecules in the biological sample.

In some embodiments, the polymerase is substantially inactive under the first temperature. In some embodiments, the polymerase that is substantially inactive under the first temperature has negligible or significantly reduced polymerase activity. In some embodiments, the polymerase that is substantially inactive under the first temperature comprises negligible or significantly reduced nucleic acid extension activity, reaction product generation, nucleic acid binding, or any combination thereof. In some embodiments, the polymerase that is substantially inactive under the first temperature remains viable but has negligible or significantly reduced nucleic acid extension activity, reaction product generation, nucleic acid binding, or any combination thereof. In some embodiments, the polymerase is more active under the second temperatures than under the first temperature. In some embodiments, the presence and/or absence of dNTPs and/or NTPs and/or derivatives and/or analogs thereof can influence the rate of polymerase activity. In some embodiments, the presence and/or absence of a cofactor of the polymerase (e.g., Mg²⁺) can influence the rate of polymerase activity. In some embodiments, the presence and/or absence of a metal ion that is not a cofactor of the polymerase (e.g., Ca²⁺) can influence the rate of polymerase activity. In some embodiments, changing one or more components of the reaction mixture can be used in part to synchronize polymerase activity and RCA reactions.

In some embodiments, the proteinase is a polymerase inhibitor that can be modulated by its presence and absence in the biological sample. In some embodiments, the proteinase comprises one or more serine protease, cysteine protease, threonine protease, aspartic protease, guamic protease, metalloprotease, and/or asparagine peptide lyase. In some embodiments, the proteinase consists of proteinase K. In some embodiments, the limited polymerase activity under the first temperature allows polymerase activity to be synchronized prior to switching to a second temperature that is permissive for polymerase activity. In some embodiments, contacting the biological sample with the proteinase under the second temperature synchronizes the conclusion of polymerase activity in the biological sample. In some embodiments, modifying the presence and absence of the proteinase results in synchronized stoppage of the polymerase activity and more homogenous RCP in terms of size and intensity.

In some embodiments, the proteinase is inactivated by incubating the biological sample at about 90° C. for about 5 seconds, about 10 seconds, about 20 seconds, about 30 seconds, about 1 minute, about 5 minutes, about 10 minutes, about 20 minutes, about 30 minutes or longer. In some embodiments, the proteinase is inactivated by treating the biological sample with an agent that inactivates the proteinase. In some embodiments, the proteinase is inactivated by treating the biological sample with an proteinase inhibitor. In some embodiments, the agent that inactivates the proteinase is a serine proteinase inhibitor. In some embodiments, the agent comprises phenylmethylsulfonyl fluoride (PMSF), diisopropyl fluorophosphate (DFP), and/or 4-benzenesulfonyl fluoride hydrochloride (AEBSF). In some embodiments, the proteinase is inactivated by treating the biological sample with one or more serine proteinase inhibitors for about 1 minute, about 5 minutes, about 10 minutes, about 20 minutes, about 30 minutes or longer. In some embodiments, the proteinase is inactivated by incubating the biological sample at about 90° C. for at least about 30 seconds, at least about 1 minute, at least about 5 minutes, or longer. In some embodiments, molecules of the polymerase in the biological sample are irreversibly inactivated and/or degraded. In some embodiments, all or nearly all of the molecules of the polymerase in the biological sample are irreversibly inactivated and/or degraded.

A biological sample having been treated to inactivate or degrade all of the molecules of the polymerase can be subsequently contacted with new molecules of polymerase. In doing so, the RCA reaction can be resumed in a deliberate and synchronized manner, thus preserving the homogeneity of RCPs produced from the polymerase-driven RCA reactions. In some embodiments, the method further comprises contacting the biological sample with additional molecules of the polymerase after lowering the temperature to a third temperature. In some embodiments, the additional molecules of the polymerase are substantially inactive under a third temperature. In some embodiments, the biological sample and the additional molecules of the polymerase are incubated under the third temperature for about 1 minute, about 5 minutes, about 10 minutes, about 20 minutes, about 30 minutes, about 40 minutes, about 50 minutes, about 1 hour, about 2 hours, about 6 hours, or longer. In some embodiments, lowering the temperature of the biological sample before contacting with additional molecules of the polymerase limits uncontrolled polymerase activity in the biological sample. In some embodiments, lowering the temperature of the biological sample before contacting with additional molecules of the polymerase permits the polymerase to complex with the nucleotide-based substrate across the biological sample without permitting polymerase activity. In some embodiments, lowering the temperature of the biological sample before contacting with additional molecules of the polymerase helps to synchronize polymerase activity across the biological sample to improve reaction product (e.g., RCP) homogeneity in terms of size and intensity to improve overall signal output (e.g., for in situ analysis).

In some embodiments, the method further comprises performing RCA in the biological sample using the polymerase under one or more fourth temperatures higher than the first and/or third temperatures. In some embodiments, the polymerase is more active under the one or more fourth temperatures than under the first and/or third temperatures. In some embodiments, the one or more fourth temperatures are the same as the one or more second temperatures. In any of the proceeding embodiments, the one fourth temperature is about 37° C. and the subsequent fourth temperature is about 60° C. In some embodiments, the method comprises temperatures cycling as a means of modulating and synchronizing polymerase activity. In some embodiments, the synchronization of polymerase activity from using different temperatures results in an increase in homogeneity of RCPs. In some embodiments, more homogenous RCPs in terms of RCP size and intensity reduce optical crowding, increase the number of RCP signals observed, and improve overall RCP signal in the biological sample.

(iv) Deoxynucleoside Triphosphate (dNTP) Limitation

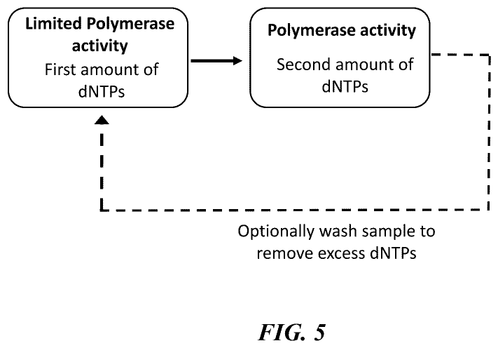

As described above, temperature regulation is an effective means of influencing polymerase activity for the purposes of synchronizing polymerase activity and RCA reactions. Another form of polymerase activity regulation includes deoxynucleoside (dNTP) limitation, which are essential substrates for the polymerization reaction. In some embodiments, providing a first amount of dNTPs that is limiting and then providing a second amount of dNTPs that is not limiting is used to synchronize polymerase activity and RCA reactions (e.g., as shown in FIG. 5). For example, contacting the biological sample comprising the polymerase with a first amount of dNTPs, wherein the first amount of dNTPs is substantially low, can limit and synchronize polymerase activity in a biological sample. Subsequent contact with the second amount of dNTPs can acutely initiate polymerase activity in a synchronized manner. Similar to the methods described in previous sections, controlling the amount of available substrate can be used to directly influence polymerase activity initiation. In some embodiments, provided herein is a method for analyzing a biological sample, comprising contacting the biological sample with a polymerase and a first amount of dNTPs comprising one or more bases, wherein the biological sample comprises a plurality of circular nucleic acid molecules and the polymerase is capable of rolling circle amplification (RCA) of the plurality of circular nucleic acid molecules. In some embodiments, the method further comprises performing RCA in the biological sample using the polymerase until the first amount of dNTPs is exhausted. In some embodiments, the method further comprises contacting the biological sample with a second amount of dNTPs comprising all four of A, T, C, and G bases or variants or analogs thereof. In some embodiments, the method further comprises performing RCA in the biological sample using the polymerase and the second amount of dNTPs. In some embodiments, the method further comprises detecting signals associated with RCA products of the plurality of circular nucleic acid molecules in the biological sample.

In some embodiments, the first amount of dNTPs comprise all four of A, T, C, and G bases and/or derivatives and/or analogs thereof. In some embodiments, the first amount of dNTPs does not comprise any one, two, or three of A, T, C, and G bases and/or derivatives and/or analogs thereof. In some embodiments, the first amount of dNTPs is less than about 1 nM, about 10 nM, about 25 nM, about 50 nM, about 100 nM, about 200 nM, about 500 nM, about 1 μM, about 5 μM, less than about 50 μM, or less than about 100 μM. In some embodiments, the first amount of dNTPs is exhausted in less than about 15 seconds, less than about 30 seconds, less than about 45 seconds, less than about 1 minute, less than about 5 minutes, less than about 10 minutes, less than about 15 minutes, less than about 20 minutes, less than about 25 minutes, or less than about 30 minutes from the initiation of RCA.

In some embodiments, the second amount of dNTPs is more than about 100 μM, more than about 150 μM, more than about 200 μM, more than about 250 μM, or more than about 300 μM, more than about 400 μM, more than about 500 μM, more than about 750 μM, or more than about 1000 μM. In some embodiments, the RCA in step d) is performed for more than about 10 minutes, more than about 20 minutes, more than about 30 minutes, more than about 40 minutes, more than about 50 minutes, more than about 1 hour, more than about 1.5 hours, more than about 2 hours, more than about 2.5 hours, more than about 3 hours, or more than about 6 hours without exhausting the second amount of dNTPs.

In some aspects, during the amplification step, modified nucleotides can be added to the reaction to incorporate the modified nucleotides in the amplification product (e.g., nanoball). Exemplary of the modified nucleotides comprise amine-modified nucleotides. In some aspects of the methods, for example, for anchoring or cross-linking of the generated amplification product (e.g., nanoball) to a scaffold, to cellular structures and/or to other amplification products (e.g., other nanoballs). In some aspects, the amplification products comprises a modified nucleotide, such as an amine-modified nucleotide. In some embodiments, the amine-modified nucleotide comprises an acrylic acid N-hydroxysuccinimide moiety modification. Examples of other amine-modified nucleotides comprise, but are not limited to, a 5-Aminoallyl-dUTP moiety modification, a 5-Propargylamino-dCTP moiety modification, a N6-6-Aminohexyl-dATP moiety modification, or a 7-Deaza-7-Propargylamino-dATP moiety modification.

### III. Nucleic Acid Probes

In some embodiments, a circular nucleic acid molecule used as a template for RCA to generate an RCA product according to the present disclosure is a circular nucleic acid probe that hybridizes to an endogenous analyte, labeling agent, or product thereof in the biological sample. In some embodiments, the circular nucleic acid molecule is generated using a circularizable probe or probe set. For example, in some cases the circular nucleic acid molecule is generated from a circularizable probe or probe set by ligation in situ in the biological sample. In some embodiments, detecting signals associated an RCA product of a circular nucleic acid molecule in the biological sample comprises contacting the biological sample with a probe that hybridizes to the RCA product, wherein the probe is associated with a signal, thereby associating the signal with the RCA product, and detecting the signal. Thus, provided herein in some aspects are nucleic acid probes and/or probe sets that are introduced into a cell or used to otherwise contact a biological sample such as a tissue sample.

The probes may comprise any of a variety of entities that can hybridize to a nucleic acid, typically by Watson-Crick base pairing, such as DNA, RNA, LNA, PNA, etc. The nucleic acid probe typically contains a targeting sequence that is able to directly or indirectly bind to at least a portion of a target nucleic acid. The nucleic acid probe may be able to bind to a specific target nucleic acid (e.g., an mRNA, or other nucleic acids as discussed herein). In some embodiments, the nucleic acid probes may be detected using a detectable label, and/or by using secondary nucleic acid probes able to bind to the nucleic acid probes. In some embodiments, the nucleic acid probes (e.g., primary probes and/or secondary probes) are compatible with one or more biological and/or chemical reactions. For instance, a nucleic acid probe disclosed herein can serve as a template or primer for a polymerase, a template or substrate for a ligase, a substrate for a click chemistry reaction, and/or a substrate for a nuclease (e.g., endonuclease or exonuclease for cleavage or digestion).

The probes that hybridize to a RCA product herein are referred to as secondary probes, and probes that hybridize to the secondary probes or to probes hybridized thereto are referred to as higher order probes. In some embodiments, more than one type of secondary probe may be contacted with a biological sample, e.g., simultaneously or sequentially in any suitable order, such as in sequential probe hybridization/unhybridization cycles. In some embodiments, more than one type of higher order nucleic acid probes may be contacted with a sample, e.g., simultaneously or sequentially in any suitable order, such as in sequential probe hybridization/unhybridization cycles. In some embodiments, the secondary probes are detectably labeled probes (comprising a detectable moiety that produces the signal). In some embodiments, any of the higher order probes may be detectably labeled probes (comprising a detectable moiety that produces the signal).

In some embodiments, at least 2, at least 5, at least 10, at least 25, at least 50, at least 75, at least 100, at least 300, at least 1,000, at least 3,000, at least 10,000, at least 30,000, at least 50,000, at least 100,000, at least 250,000, at least 500,000, or at least 1,000,000 distinguishable circular or circularizable nucleic acid probes or probe sets are contacted with a sample, e.g., simultaneously or sequentially in any suitable order. In some embodiments, the circular or circularizable probes are distinguished based on one or more barcode sequences or complements thereof in the probes, wherein the complementary sequence (barcode sequence) in the RCA product generated from the circular or circularizable probe can be detected to distinguish the probes.

Between any of the probe contacting steps disclosed herein, the method may comprise one or more intervening reactions and/or processing steps, such as modifications of a target nucleic acid, modifications of a probe or product thereof (e.g., via hybridization, ligation, extension, amplification, cleavage, digestion, branch migration, primer exchange reaction, click chemistry reaction, crosslinking, attachment of a detectable label, activating photo-reactive moieties, etc.), removal of a probe or product thereof (e.g., cleaving off a portion of a probe and/or unhybridizing the entire probe), signal modifications (e.g., quenching, masking, photo-bleaching, signal enhancement (e.g., via FRET), signal amplification, etc.), signal removal (e.g., cleaving off or permanently inactivating a detectable label), crosslinking, de-crosslinking, and/or signal detection.

The target-binding sequence (sometimes also referred to as the targeting region/sequence or the recognition region/sequence) of a probe may be positioned anywhere within the probe. For instance, the target-binding sequence of a primary probe that binds to a target nucleic acid can be 5′ or 3′ to any barcode sequence in the primary probe. Likewise, the target-binding sequence of a secondary probe (which binds to a primary probe or complement or product thereof) can be 5′ or 3′ to any barcode sequence in the secondary probe. In some embodiments, the target-binding sequence may comprise a sequence that is substantially complementary to a portion of a target nucleic acid. In some embodiments, the portions may be at least 50%, at least 60%, at least 70%, at least 75%, at least 80%, at least 85%, at least 90%, at least 92%, at least 94%, at least 95%, at least 96%, at least 97%, at least 98%, at least 99%, or 100% complementary.

The target-binding sequence of a circular or circularizable probe or probe set may be determined with reference to a target nucleic acid (e.g., a cellular RNA or product thereof, or a probe or reporter oligonucleotide of a labeling agent for a cellular analyte) that is present or suspected of being present in a sample. In some embodiments, more than one target-binding sequence can be used to identify a particular analyte comprising or associated with a target nucleic acid. The more than one target-binding sequence can be in the same probe or in different probes. For instance, multiple probes can be used, sequentially and/or simultaneously, that can bind to (e.g., hybridize to) different regions of the same target nucleic acid. In other examples, a probe may comprise target-binding sequences that can bind to different target nucleic acid sequences, e.g., various intron and/or exon sequences of the same gene (for detecting splice variants, for example), or sequences of different genes, e.g., for detecting a product that comprises the different target nucleic acid sequences, such as a genome rearrangement (e.g., inversion, transposition, translocation, insertion, deletion, duplication, and/or amplification).

After contacting the nucleic acid probes with a sample, the probes may be directly detected by determining detectable labels (if present), and/or detected by using one or more other probes that bind directly or indirectly to the probes or products thereof. The one or more other probes may comprise a detectable label. For instance, a primary nucleic acid probe can bind to a target nucleic acid in the sample, and a secondary nucleic acid probe can be introduced to bind to an amplification product of the primary nucleic acid probe, where the secondary nucleic acid probe or a product thereof can then be detected using detectably labeled probes. Higher order probes that directly or indirectly bind to the secondary nucleic acid probe or product thereof may also be used, and the higher order probes or products thereof can then be detected using detectably labeled probes.

In some embodiments, the detection may be spatial, e.g., in two or three dimensions. In some embodiments, the detection may be quantitative, e.g., the amount or concentration of a primary nucleic acid probe (and of a target nucleic acid) may be determined. In some embodiments, the primary probes, secondary probes, higher order probes, and/or detectably labeled probes may comprise any of a variety of entities able to hybridize a nucleic acid, e.g., DNA, RNA, LNA, and/or PNA, etc., depending on the application.

A secondary nucleic acid probe may contain a recognition sequence able to bind to or hybridize with an RCA product e.g., at a barcode sequence or portion(s) thereof of the RCA product. In some embodiments, a secondary nucleic acid probe may bind to a combination of barcode sequences (which may be continuous or spaced from one another) in a RCA product. In some embodiments, the binding is specific, or the binding may be such that a recognition sequence preferentially binds to or hybridizes with only one of the barcode sequences or complements thereof that are present. The secondary nucleic acid probe may also contain one or more detectable labels. If more than one secondary nucleic acid probe is used, the detectable labels may be the same or different.

The recognition sequences may be of any length, and multiple recognition sequences in the same or different secondary nucleic acid probes may be of the same or different lengths. If more than one recognition sequence is used, the recognition sequences may independently have the same or different lengths. For instance, the recognition sequence may be at least 4, at least 5, least 6, least 7, least 8, least 9, at least 10, least 11, least 12, least 13, least 14, at least 15, least 16, least 17, least 18, least 19, at least 20, at least 25, at least 30, at least 35, at least 40, or at least 50 nucleotides in length. In some embodiments, the recognition sequence may be no more than 48, no more than 40, no more than 32, no more than 24, no more than 16, no more than 12, no more than 10, no more than 8, or no more than 6 nucleotides in length. Combinations of any of these are also possible, e.g., the recognition sequence may have a length of between 5 and 8, between 6 and 12, or between 7 and 15 nucleotides, etc. In one embodiment, the recognition sequence is of the same length as a barcode sequence or complement thereof of a primary nucleic acid probe or a product thereof. In some embodiments, the recognition sequence may be at least 50%, at least 60%, at least 70%, at least 75%, at least 80%, at least 85%, at least 90%, at least 92%, at least 94%, at least 95%, at least 96%, at least 97%, at least 98%, at least 99%, or at least 100% complementary to the barcode sequence or complement thereof.

The barcode sequences, if present, may be of any length. If more than one barcode sequence is used, the barcode sequences may independently have the same or different lengths, such as at least 5, at least 10, at least 15, at least 20, at least 25, at least 30, at least 35, at least 40, at least 50 nucleotides in length. In some embodiments, the barcode sequence may be no more than 120, no more than 112, no more than 104, no more than 96, no more than 88, no more than 80, no more than 72, no more than 64, no more than 56, no more than 48, no more than 40, no more than 32, no more than 24, no more than 16, or no more than 8 nucleotides in length. Combinations of any of these are also possible, e.g., the barcode sequence may be between 5 and 10 nucleotides, between 8 and 15 nucleotides, etc.

The barcode sequence may be arbitrary or random. In certain cases, the barcode sequences are chosen so as to reduce or minimize homology with other components in a sample, e.g., such that the barcode sequences do not themselves bind to or hybridize with other nucleic acids suspected of being within the cell or other sample. In some embodiments, between a particular barcode sequence and another sequence (e.g., a cellular nucleic acid sequence in a sample or other barcode sequences in probes added to the sample), the homology may be less than 10%, less than 8%, less than 7%, less than 6%, less than 5%, less than 4%, less than 3%, less than 2%, or less than 1%. In some embodiments, the homology may be less than 20, 19, 18, 17, 16, 15, 14, 13, 12, 11, 10, 9, 8, 7, 6, 5, 4, 3, or 2 bases, and in some embodiments, the bases are consecutive bases.

In some embodiments, the nucleic acid probes disclosed herein may be made using only 2 or only 3 of the 4 bases, such as leaving out all the “G”s and/or leaving out all of the “C”s within the probe. Sequences lacking either “G”s or “C”s may form very little secondary structure, and can contribute to more uniform, faster hybridization in certain embodiments.

In some embodiments, a nucleic acid probe disclosed herein may contain a detectable label such as a fluorophore. In some embodiments, one or more probes of a plurality of nucleic acid probes used in an assay may lack a detectable label, while one or more other probes in the plurality each comprises a detectable label selected from a limited pool of distinct detectable labels (e.g., red, green, yellow, and blue fluorophores), and the absence of detectable label may be used as a separate “color.” As such, detectable labels are not required in all cases. In some embodiments, a primary nucleic acid probe (e.g., a circularizable probe such as a padlock probe) disclosed herein lacks a detectable label. While a detectable label may be incorporated into an amplification product of the primary nucleic acid probe, such as via incorporation of a modified nucleotide into an RCA product of a circularizable probe, the amplification product in some embodiments is not detectably labeled. In some embodiments, a probe that binds to the primary nucleic acid probe or a product thereof (e.g., a secondary nucleic acid probe that binds to a barcode sequence or complement thereof in the primary nucleic acid probe or product thereof) comprises a detectable label and may be used to detect the primary nucleic acid probe or product thereof. In some embodiments, a secondary nucleic acid probe disclosed herein lacks a detectable label, and a detectably labeled probe that binds to the secondary nucleic acid probe or a product thereof (e.g., at a barcode sequence or complement thereof in the secondary nucleic acid probe or product thereof) can be used to detect the second nucleic acid probe or product thereof. In some embodiments, signals associated with the detectably labeled probes can be used to detect one or more barcode sequences in the secondary probe and/or one or more barcode sequences in the primary probe, e.g., by using sequential hybridization of detectably labeled probes, sequencing-by-ligation, and/or sequencing-by-hybridization. In some embodiments, the barcode sequences (e.g., in the secondary probe and/or in the primary probe) are used to combinatorially encode a plurality of analytes of interest. As such, signals associated with the detectably labeled probes at particular locations in a biological sample can be used to generate distinct signal signatures that each corresponds to an analyte in the sample, thereby identifying the analytes at the particular locations, e.g., for in situ spatial analysis of the sample.

In some embodiments, a nucleic acid probe herein comprises one or more other components, such as one or more primer binding sequences (e.g., to allow for enzymatic amplification of probes), enzyme recognition sequences (e.g., for endonuclease cleavage), or the like. The components of the nucleic acid probe may be arranged in any suitable order

In some aspects, a high fidelity ligase, such as a thermostable DNA ligase (e.g., a Taq DNA ligase), is used. Thermostable DNA ligases are active at elevated temperatures, allowing further discrimination by incubating the ligation at a temperature near the melting temperature (Tₘ) of the DNA strands. This selectively reduces the concentration of annealed mismatched substrates (expected to have a slightly lower Tₘ around the mismatch) over annealed fully base-paired substrates. Thus, high-fidelity ligation can be achieved through a combination of the intrinsic selectivity of the ligase active site and balanced conditions to reduce the incidence of annealed mismatched dsDNA.

In some embodiments, a probe that hybridizes to an RCA product comprises a target recognition sequence that hybridizes to a sequence of the RCA product (e.g., a barcode sequence in the RCA product) and a sequence that does not hybridize the RCA product, such as a 5′ overhang and/or a 3′ overhang. In some embodiments, the sequence (e.g., the 5′ overhang, 3′ overhang, and/or linker or spacer) hybridizes to another probe, such as a detectably labeled probes, thereby associating the probe with a signal produced by the detectably labeled probe.

Any suitable circularizable probe or probe set may be used to generate the RCA template which is used to generate the RCA product. In some instances, the circularizable probe or probe set is in the form of a linear nucleic acid molecule or set of linear nucleic acid molecules having ligatable ends which may be circularized by ligating the ends together directly or indirectly, e.g. to each other. In some embodiments, the linear nucleic acid molecule or set of molecules are circularized by ligating the ligatable ends of an intervening (“gap”) oligonucleotide to ends of the linear nucleic acid molecule(s). In some embodiments, an end of the linear nucleic acid molecule is extended by a polymerase (e.g., using a target nucleic acid as a template), and the extended end of the linear nucleic acid molecule can be ligated to another end of the linear nucleic acid molecule or set of molecules to generate a circular nucleic acid molecule. A circularizable template may be provided in two or more parts, namely two or more molecules (e.g. oligonucleotides) which may be ligated together to form a circle. When said RCA template is circularizable it is circularized by ligation prior to RCA. Ligation may be templated using a ligation template, and in the case of a circularizable probe or probe set, e.g., padlock and molecular inversion probes and such like the target analyte may provide the ligation template, or it may be separately provided. The circularizable RCA template (or template part or portion) will comprise at its respective 3′ and 5′ ends regions of complementarity to corresponding cognate complementary regions (or binding sites) in the ligation template, which may be adjacent where the ends are directly ligated to each other, or non-adjacent, with an intervening “gap” sequence, where indirect ligation is to take place.

In some embodiments, the ligation involves chemical ligation (e.g., click chemistry ligation). In some embodiments, the chemical ligation involves template dependent ligation. In some embodiments, the chemical ligation involves template independent ligation. In some embodiments, the click reaction is a template-independent reaction (see, e.g., Xiong and Seela (2011), J. Org. Chem. 76(14): 5584-5597, incorporated by reference herein in its entirety). In some embodiments, the click reaction is a template-dependent reaction or template-directed reaction. In some embodiments, the template-dependent reaction is sensitive to base pair mismatches such that reaction rate is significantly higher for matched versus unmatched templates. In some embodiments, the click reaction is a nucleophilic addition template-dependent reaction. In some embodiments, the click reaction is a cyclopropane-tetrazine template-dependent reaction

In some embodiments, the ligation involves enzymatic ligation. In some embodiments, the enzymatic ligation involves use of a ligase. In some aspects, the ligase used herein comprises an enzyme that is commonly used to join polynucleotides together or to join the ends of a single polynucleotide. An RNA ligase, a DNA ligase, or another variety of ligase can be used to ligate two nucleotide sequences together. Ligases comprise ATP-dependent double-strand polynucleotide ligases, NAD-i-dependent double-strand DNA or RNA ligases and single-strand polynucleotide ligases, for example any of the ligases described in EC 6.5.1.1 (ATP-dependent ligases), EC 6.5.1.2 (NAD+-dependent ligases), EC 6.5.1.3 (RNA ligases). Specific examples of ligases comprise bacterial ligases such as E. coli DNA ligase, Tth DNA ligase, Thermococcus sp. (strain 9° N) DNA ligase (9° N™ DNA ligase, New England Biolabs), Taq DNA ligase, Ampligase™ (Epicentre Biotechnologies) and phage ligases such as T3 DNA ligase, T4 DNA ligase and T7 DNA ligase and mutants thereof. In some embodiments, the ligase is a T4 RNA ligase. In some embodiments, the ligase is a splintR ligase. In some embodiments, the ligase is a single stranded DNA ligase. In some embodiments, the ligase is a T4 DNA ligase. In some embodiments, the ligase is a ligase that has an DNA-splinted DNA ligase activity. In some embodiments, the ligase is a ligase that has an RNA-splinted DNA ligase activity.

In some embodiments, the ligation herein is a direct ligation. In some embodiments, the ligation herein is an indirect ligation. “Direct ligation” means that the ends of the polynucleotides hybridize immediately adjacently to one another to form a substrate for a ligase enzyme resulting in their ligation to each other (intramolecular ligation). Alternatively, “indirect” means that the ends of the polynucleotides hybridize non-adjacently to one another, e.g., separated by one or more intervening nucleotides or “gaps”. In some embodiments, said ends are not ligated directly to each other, but instead occurs either via the intermediacy of one or more intervening (so-called “gap” or “gap-filling” (oligo)nucleotides) or by the extension of the 3′ end of a probe to “fill” the “gap” corresponding to said intervening nucleotides (intermolecular ligation). In some cases, the gap of one or more nucleotides between the hybridized ends of the polynucleotides may be “filled” by one or more “gap” (oligo)nucleotide(s) which are complementary to a splint, a circularizable probe or probe set (e.g., a padlock probe), or target nucleic acid. The gap may be a gap of 1 to 60 nucleotides or a gap of 1 to 40 nucleotides or a gap of 3 to 40 nucleotides. In specific embodiments, the gap may be a gap of about 1, 2, 3, 4, 5, 6, 7, 8, 9 or 10 or more nucleotides, of any integer (or range of integers) of nucleotides in between the indicated values. In some embodiments, the gap between said terminal regions may be filled by a gap oligonucleotide or by extending the 3′ end of a polynucleotide. In some cases, ligation involves ligating the ends of the probe to at least one gap (oligo)nucleotide, such that the gap (oligo)nucleotide becomes incorporated into the resulting polynucleotide. In some embodiments, the ligation herein is preceded by gap filling. In other embodiments, the ligation herein does not require gap filling.

In the case of circularizable probes or probe sets (e.g., padlock probes), in one embodiment the ends of the circularizable probe or probe set may be brought into proximity to each other by hybridization to adjacent sequences on a target nucleic acid molecule (such as a target analyte), which acts as a ligation template, thus allowing the ends to be ligated together to form a circular nucleic acid molecule, allowing the circularized probe or probe set to act as a template for an RCA reaction. In such an example, the terminal sequences of the circularizable probe or probe set, which hybridize to the target nucleic acid molecule, will be specific to the target analyte in question, and will be replicated repeatedly in the RCA product. They may therefore act as a marker sequence indicative of that target analyte. Accordingly, it can be seen that the marker sequence in the RCA product may be equivalent to a sequence present in the target analyte itself. Alternatively, a marker sequence (e.g. tag or barcode sequence) may be provided in the non-target complementary parts of the circularizable probe or probe set. In still a further embodiment, the marker sequence may be present in the gap oligonucleotide which is hybridized between the respective hybridized ends of the circularizable probe or probe set, where they are hybridized to non-adjacent sequences in the target molecule. Such gap-filling circularizable probe or probe sets are akin to molecular inversion probes.

In some embodiments, similar circular RCA template molecules can be generated using molecular inversion probes. Like circularizable probes, these are also typically linear nucleic acid molecules capable of hybridizing to a target nucleic acid molecule (such as a target analyte) and being circularized. The two ends of the molecular inversion probe may hybridize to the target nucleic acid molecule at sites which are proximate but not directly adjacent to each other, resulting in a gap between the two ends. The size of this gap may range from only a single nucleotide in some embodiments, to larger gaps of 100 to 500 nucleotides, or longer, in other embodiments. Accordingly, it is necessary to supply a polymerase and a source of nucleotides, or an additional gap-filling oligonucleotide, in order to fill the gap between the two ends of the molecular inversion probe, such that it can be circularized.

As with the circularizable probes or probe sets, the terminal sequences of the molecular inversion probe which hybridize to the target nucleic acid molecule, and the sequence between them, will be specific to the target analyte in question, and will be replicated repeatedly in the RCA product. They may therefore act as a marker sequence indicative of that target analyte. Alternatively, a marker sequence (e.g. tag or barcode sequence) may be provided in the non-target complementary parts of the molecular inversion probe.

In some embodiments, the probes disclosed herein may be invader probes, e.g., for generating a circular nucleic acid such as a circularized probe. Such probes are of particular utility in the detection of single nucleotide polymorphisms. The detection method of the present invention may, therefore, be used in the detection of a single nucleotide polymorphism, or indeed any variant base, in the target nucleic acid sequence. Probes for use in such a method may be designed such that the 3′ ligatable end of the probe is complementary to and capable of hybridizing to the nucleotide in the target molecule which is of interest (the variant nucleotide), and the nucleotide at the 3′ end of the 5′ additional sequence at the 5′ end of the probe or at the 5′ end of another, different, probe part is complementary to the same said nucleotide, but is prevented from hybridizing thereto by a 3′ ligatable end (e.g., it is a displaced nucleotide). Cleavage of the probe to remove the additional sequence provides a 5′ ligatable end, which may be ligated to the 3′ ligatable end of the probe or probe part if the 3′ ligatable end is hybridized correctly to (e.g. is complementary to) the target nucleic acid molecule. Probes designed according to this principle provide a high degree of discrimination between different variants at the position of interest, as only probes in which the 3′ ligatable end is complementary to the nucleotide at the position of interest may participate in a ligation reaction. In one embodiment, the probe is provided in a single part, and the 3′ and 5′ ligatable ends are provided by the same probe. In some embodiments, an invader probe is a circularizable probe (e.g., an invader padlock or “iLock”), e.g., as described in Krzywkowski et al., Nucleic Acids Research 45, e161, 2017 and US 2020/0224244, which are incorporated herein by reference in their entireties.

Other types of probe which result in circular molecules which can be detected by RCA and which comprise either a target analyte sequence or a complement thereof include selector-type probes described in US20190144940, which comprise sequences capable of directing the cleavage of a target nucleic acid molecule (e.g. a target analyte) so as to release a fragment comprising a target sequence from the target analyte and sequences capable of templating the circularization and ligation of the fragment. U.S. Pat. No. 11,352,658, the content or which is herein incorporated by reference in its entirety, describes probes which comprise a 3′ sequence capable of hybridizing to a target nucleic acid molecule (e.g. a target analyte) and acting as a primer for the production of a complement of a target sequence within the target nucleic acid molecule (e.g. by target templated extension of the primer), and an internal sequence capable of templating the circularization and ligation of the extended probe comprising the reverse complement of the target sequence within the target analyte and a portion of the probe. In the case of both such probes, target sequences or complements thereof are incorporated into a circularized molecule which acts as the template for the RCA reaction to generate the RCA product, which consequently comprises concatenated repeats of said target sequence. In some embodiments, said target sequence may act as, or may comprise a marker sequence within the RCA product indicative of the target analyte in question. Alternatively, a marker sequence (e.g. tag or barcode sequence) may be provided in the non-target complementary parts of the probes.

In some embodiments, a nucleic acid probe disclosed herein can be pre-assembled from multiple components, e.g., prior to contacting the nucleic acid probe with a target nucleic acid or a sample. In some embodiments, a nucleic acid probe disclosed herein can be assembled during and/or after contacting a target nucleic acid or a sample with multiple components. In some embodiments, a nucleic acid probe disclosed herein is assembled in situ in a sample. In some embodiments, the multiple components can be contacted with a target nucleic acid or a sample in any suitable order and any suitable combination. For instance, a first component and a second component can be contacted with a target nucleic acid, to allow binding between the components and/or binding between the first and/or second components with the target nucleic acid. Optionally a reaction involving either or both components and/or the target nucleic acid, between the components, and/or between either one or both components and the target nucleic acid can be performed, such as hybridization, ligation, primer extension and/or amplification, chemical or enzymatic cleavage, click chemistry, or any combination thereof. In some embodiments, a third component can be added prior to, during, or after the reaction. In some embodiments, a third component can be added prior to, during, or after contacting the sample with the first and/or second components. In some embodiments, the first, second, and third components can be contacted with the sample in any suitable combination, sequentially or simultaneously. In some embodiments, the nucleic acid probe can be assembled in situ in a stepwise manner, each step with the addition of one or more components, or in a dynamic process where all components are assembled together. One or more removing steps, e.g., by washing the sample such as under stringent conditions, may be performed at any point during the assembling process to remove or destabilize undesired intermediates and/or components at that point and increase the chance of accurate probe assembly and specific target binding of the assembled probe.

In some aspects, the polynucleotides and/or amplification product (e.g., amplicon) can be anchored to a polymer matrix. For example, the polymer matrix can be a hydrogel. In some embodiments, one or more of the polynucleotide probe(s) can be modified to contain functional groups that can be used as an anchoring site to attach the polynucleotide probes and/or amplification product to a polymer matrix. Exemplary modification and polymer matrix that can be employed in accordance with the provided embodiments comprise those described in, for example, U.S. Pat. Nos. 10,138,509, 11,542,554, U.S. 2016/0024555, U.S. 2018/0251833 and U.S. 2017/0219465, which are herein incorporated by reference in their entireties. In some examples, the scaffold also contains modifications or functional groups that can react with or incorporate the modifications or functional groups of the probe set or amplification product. In some examples, the scaffold can comprise oligonucleotides, polymers or chemical groups, to provide a matrix and/or support structures.

In some cases, following formation of the circular nucleic acid, in some instances, an amplification primer is added for RCA. In other instances, the amplification primer is added with the primary and/or secondary probes. In some instances, the amplification primer may also be complementary to the target nucleic acid and the circularizable probe (e.g., a SNAIL probe). In some embodiments, a washing step is performed to remove any unbound probes, primers, etc. In some embodiments, the wash is a stringency wash. Washing steps can be performed at any point during the process to remove non-specifically bound probes, probes that have ligated, etc.

### IV. Signal Amplification, Detection and Analysis

In some aspects, the provided methods involve analyzing, e.g., detecting or determining, one or more sequences present in the probes (e.g., described in Section III) and/or in a reaction product or derivative thereof, such as in an amplification product. In some cases, the analysis is performed on one or more images captured, and may comprise processing the image(s) and/or quantifying signals observed. For example, the analysis may comprise processing information of one or more cell types, one or more types of biomarkers, a number or level of a biomarker, and/or a number or level of cells detected in a particular region of the sample. In some embodiments, the analysis comprises detecting a sequence (e.g., a barcode present in the sample). In some embodiments, the analysis includes quantification of puncta (e.g., if amplification products are detected). In some cases, the analysis includes determining whether particular cells and/or signals are present that correlate with one or more biomarkers from a particular panel. In some embodiments, the obtained information may be compared to a positive and negative control, or to a threshold of a feature to determine if the sample exhibits a certain feature or phenotype. In some cases, the information may comprise signals from a cell, a region, and/or comprise readouts from multiple detectable labels. In some case, the analysis further includes displaying the information from the analysis or detection step. In some embodiments, software may be used to automate the processing, analysis, and/or display of data.

In some embodiments detecting signals associated with RCA products of the plurality of circular nucleic acid molecules in the biological sample comprises probes to the RCA products, wherein the probes are associated with signals. In some embodiments, the probes are detectably labeled probes. In some aspects, a signal associated with a probe that hybridizes to an RCA product herein is amplified in situ in the biological sample or in a matrix embedding the biological sample.

In some embodiments, the analysis comprises determining the sequence of all or a portion of the amplification product. In some embodiments, the analysis comprises detecting a sequence present in the amplification product. In some embodiments, the sequence of all or a portion of the amplification product is indicative of the identity of a region of interest in a target nucleic acid. In other embodiments, the provided methods involve analyzing, e.g., detecting or determining, one or more sequences present in the polynucleotide probes (e.g., a barcode sequence).

In some embodiments, the methods comprise sequencing all or a portion of the amplification product, such as one or more barcode sequences present in the amplification product. In some embodiments, the analysis and/or sequence determination comprises sequencing all or a portion of the amplification product or the probe(s) and/or in situ hybridization to the amplification product or the probe(s). In some embodiments, the sequencing step involves sequencing by hybridization, sequencing by ligation, and/or fluorescent in situ sequencing, hybridization-based in situ sequencing and/or wherein the in situ hybridization comprises sequential fluorescent in situ hybridization. In some embodiments, the detection or determination comprises hybridizing to the amplification product (or a probe bound thereto) a detection oligonucleotide labeled with a fluorophore, an isotope, a mass tag, or any combination thereof. In some embodiments, the detection or determination comprises imaging the amplification product. In some embodiments, the target nucleic acid is an mRNA in a tissue sample, and the detection or determination is performed when the target nucleic acid and/or the amplification product is in situ in the tissue sample.

In some aspects, the provided methods comprise imaging the amplification product (e.g., amplicon) and/or one or more portions of the polynucleotides, for example, via binding of the detection probe and detecting an associated detectable label. In some embodiments, the probes (e.g., detection probes) for binding to the amplification product are L-shaped probes comprising overhang regions for hybridization of detectably labeled probes imaged to detect signals associated with the RCA products. In some embodiments, the one or more detection probes comprise one or more overhang regions (e.g., a 5′ and/or 3′ end of the probe that does not hybridize to the rolling circle amplification product). A probe comprising a single overhang region may be referred to as an “L-shaped probe,” and a probe comprising two overhangs may be referred to as a “U-shaped probe.” In some cases, the overhang region comprises a binding region for binding one or more detectably-labeled probes. In some embodiments, the detecting comprises contacting the biological sample with a pool of detection probes corresponding to different barcode sequences or portions thereof, and a pool of detectably-labeled probes corresponding to different detectable labels. In some embodiments, the biological sample is sequentially contacted with different pools of detection probes. In some instances, a common or universal pool of detectably-labeled probes is used in a plurality of sequential hybridization steps (e.g., with different pools of detection probes).

In some embodiments, the detection probe or detectably labeled probe comprises a detectable label that can be measured and quantitated. The terms “label” and “detectable label” comprise a directly or indirectly detectable moiety that is associated with (e.g., conjugated to) a molecule to be detected, e.g., a detectable probe, comprising, but not limited to, fluorophores, radioactive isotopes, fluorescers, chemiluminescers, enzymes, enzyme substrates, enzyme cofactors, enzyme inhibitors, chromophores, dyes, metal ions, metal sols, ligands (e.g., biotin or haptens) and the like.

The term “fluorophore” comprises a substance or a portion thereof that is capable of exhibiting fluorescence in the detectable range. Particular examples of labels that may be used in accordance with the provided embodiments comprise, but are not limited to phycoerythrin, Alexa dyes, fluorescein, YPet, CyPet, Cascade blue, allophycocyanin, Cy3, Cy5, Cy7, rhodamine, dansyl, umbelliferone, Texas red, luminol, acradimum esters, biotin, green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), firefly luciferase, Renilla luciferase, NADPH, beta-galactosidase, horseradish peroxidase, glucose oxidase, alkaline phosphatase, chloramphenical acetyl transferase, and urease.

Fluorescence detection in tissue samples can often be hindered by the presence of strong background fluorescence. “Autofluorescence” is the general term used to distinguish background fluorescence (that can arise from a variety of sources, including aldehyde fixation, extracellular matrix components, red blood cells, lipofuscin, and the like) from the desired immunofluorescence from the fluorescently labeled antibodies or probes. Tissue autofluorescence can lead to difficulties in distinguishing the signals due to fluorescent antibodies or probes from the general background. In some embodiments, a method disclosed herein utilizes one or more agents to reduce tissue autofluorescence, for example, Autofluorescence Eliminator (Sigma/EMD Millipore), TrueBlack Lipofuscin Autofluorescence Quencher (Biotium), MaxBlock Autofluorescence Reducing Reagent Kit (MaxVision Biosciences), and/or a very intense black dye (e.g., Sudan Black, or comparable dark chromophore).

In some embodiments, a detectable probe containing a detectable label can be used to detect one or more polynucleotide(s) and/or amplification products (e.g., amplicon) described herein. In some embodiments, the methods involve incubating the detectable probe containing the detectable label with the sample, washing unbound detectable probe, and detecting the label, e.g., by imaging.

Examples of detectable labels comprise but are not limited to various radioactive moieties, enzymes, prosthetic groups, fluorescent markers, luminescent markers, bioluminescent markers, metal particles, protein-protein binding pairs and protein-antibody binding pairs. Examples of fluorescent proteins comprise, but are not limited to, yellow fluorescent protein (YFP), green fluorescence protein (GFP), cyan fluorescence protein (CFP), umbelliferone, fluorescein, fluorescein isothiocyanate, rhodamine, dichlorotriazinylamine fluorescein, dansyl chloride and phycoerythrin.

Examples of bioluminescent markers comprise, but are not limited to, luciferase (e.g., bacterial, firefly and click beetle), luciferin, aequorin and the like. Examples of enzyme systems having visually detectable signals comprise, but are not limited to, galactosidases, glucorimidases, phosphatases, peroxidases and cholinesterases. Identifiable markers also comprise radioactive compounds such as ¹²⁵I, ³⁵S, ¹⁴C, or ³H. Identifiable markers are commercially available from a variety of sources.

Examples of fluorescent labels and nucleotides and/or polynucleotides conjugated to such fluorescent labels comprise those described in, for example, Hoagland, Handbook of Fluorescent Probes and Research Chemicals, Ninth Edition (Molecular Probes, Inc., Eugene, 2002); Keller and Manak, DNA Probes, 2nd Edition (Stockton Press, New York, 1993); Eckstein, editor, Oligonucleotides and Analogues: A Practical Approach (IRL Press, Oxford, 1991); and Wetmur, Critical Reviews in Biochemistry and Molecular Biology, 26:227-259 (1991), all of which are herein incorporated by reference in their entireties. In some embodiments, exemplary techniques and methods methodologies applicable to the provided embodiments comprise those described in, for example, U.S. Pat. Nos. 4,757,141, 5,151,507 and 5,091,519, all of which are herein incorporated by reference in their entireties. In some embodiments, one or more fluorescent dyes are used as labels for labeled target sequences, for example, as described in U.S. Pat. No. 5,188,934 (4,7-dichlorofluorescein dyes); U.S. Pat. No. 5,366,860 (spectrally resolvable rhodamine dyes); U.S. Pat. No. 5,847,162 (4,7-dichlororhodamine dyes); U.S. Pat. No. 4,318,846 (ether-substituted fluorescein dyes); U.S. Pat. No. 5,800,996 (energy transfer dyes); U.S. Pat. No. 5,066,580 (xanthine dyes); and U.S. Pat. No. 5,688,648 (energy transfer dyes), all of which are herein incorporated by reference in their entireties. Labeling can also be carried out with quantum dots, as described in U.S. Pat. Nos. 6,322,901, 6,576,291, 6,423,551, 6,251,303, 6,319,426, 6,426,513, 6,444,143, 5,990,479, 6,207,392, U.S. 2002/0045045 and U.S. 2003/0017264, all of which are herein incorporated by reference in their entireties.

Examples of commercially available fluorescent nucleotide analogues readily incorporated into nucleotide and/or polynucleotide sequences comprise, but are not limited to, Cy3-dCTP, Cy3-dUTP, Cy5-dCTP, Cy5-dUTP (Amersham Biosciences, Piscataway, N.J.), fluorescein-12-dUTP, tetramethylrhodamine-6-dUTP, TEXAS RED™-5-dUTP, CASCADE BLUE™-7-dUTP, BODIPY TMFL-14-dUTP, BODIPY TMR-14-dUTP, BODIPY TMTR-14-dUTP, RHOD AMINE GREEN™-5-dUTP, OREGON GREENR™ 488-5-dUTP, TEXAS RED™-12-dUTP, BODIPY™ 630/650-14-dUTP, BODIPY™ 650/665-14-dUTP, ALEXA FLUOR™ 488-5-dUTP, ALEXA FLUOR™ 532-5-dUTP, ALEXA FLUOR™ 568-5-dUTP, ALEXA FLUOR™ 594-5-dUTP, ALEXA FLUOR™ 546-14-dUTP, fluorescein-12-UTP, tetramethylrhodamine-6-UTP, TEXAS RED™-5-UTP, mCherry, CASCADE BLUE™-7-UTP, BODIPY™ FL-14-UTP, BODIPY TMR-14-UTP, BODIPY™ TR-14-UTP, RHOD AMINE GREEN™-5-UTP, ALEXA FLUOR™ 488-5-UTP, and ALEXA FLUOR™ 546-14-UTP (Molecular Probes, Inc. Eugene, Oreg.). Methods for custom synthesis of nucleotides having other fluorophores include those disclosed in Henegariu et al. (2000) Nature Biotechnol. 18:345, which is incorporated herein by reference in its entirety for all purposes.

Other fluorophores available for post-synthetic attachment comprise, but are not limited to, ALEXA FLUOR™ 350, ALEXA FLUOR™ 532, ALEXA FLUOR™ 546, ALEXA FLUOR™ 568, ALEXA FLUOR™ 594, ALEXA FLUOR™ 647, BODIPY 493/503, BODIPY FL, BODIPY R6G, BODIPY 530/550, BODIPY TMR, BODIPY 558/568, BODIPY 558/568, BODIPY 564/570, BODIPY 576/589, BODIPY 581/591, BODIPY 630/650, BODIPY 650/665, Cascade Blue, Cascade Yellow, Dansyl, lissamine rhodamine B, Marina Blue, Oregon Green 488, Oregon Green 514, Pacific Blue, rhodamine 6G, rhodamine green, rhodamine red, tetramethyl rhodamine, Texas Red (available from Molecular Probes, Inc., Eugene, Oreg.), Cy2, Cy3.5, Cy5.5, and Cy7 (Amersham Biosciences, Piscataway, N.J.). FRET tandem fluorophores may also be used, comprising, but not limited to, PerCP-Cy5.5, PE-Cy5, PE-Cy5.5, PE-Cy7, PE-Texas Red, APC-Cy7, PE-Alexa dyes (610, 647, 680), and APC-Alexa dyes.

In some cases, metallic silver or gold particles may be used to enhance signal from fluorescently labeled nucleotide and/or polynucleotide sequences (Lakowicz et al. Bio Techniques 2003; 34(1):62-6).

Biotin, or a derivative thereof, may also be used as a label on a nucleotide and/or a polynucleotide sequence, and subsequently bound by a detectably labeled avidin/streptavidin derivative (e.g., phycoerythrin-conjugated streptavidin), or a detectably labeled anti-biotin antibody. Digoxigenin may be incorporated as a label and subsequently bound by a detectably labeled anti-digoxigenin antibody (e.g., fluoresceinated anti-digoxigenin). An aminoallyl-dUTP residue may be incorporated into a polynucleotide sequence and subsequently coupled to an N-hydroxy succinimide (NHS) derivatized fluorescent dye. In general, any member of a conjugate pair may be incorporated into a detection polynucleotide provided that a detectably labeled conjugate partner can be bound to permit detection. In various embodiments, an antibody may refer to an antibody molecule of any class, or any sub-fragment thereof, such as a Fab.

Other suitable labels for a polynucleotide sequence may comprise fluorescein (FAM), digoxigenin, dinitrophenol (DNP), dansyl, biotin, bromodeoxyuridine (BrdU), hexahistidine (6×His), and phosphor-amino acids (e.g., P-tyr, P-ser, P-thr). In some embodiments the following hapten/antibody pairs are used for detection, in which each of the antibodies is derivatized with a detectable label: biotin/a-biotin, digoxigenin/a-digoxigenin, dinitrophenol (DNP)/a-DNP, 5-Carboxyfluorescein (FAM)/a-FAM.

In some embodiments, a nucleotide and/or an polynucleotide sequence can be indirectly labeled, especially with a hapten that is then bound by a capture agent, e.g., as disclosed in U.S. Pat. Nos. 5,344,757, 5,702,888, 5,354,657, 5,198,537 and 4,849,336, and PCT publication WO 91/17160, all of which are herein incorporated by reference in their entireties. Many different hapten-capture agent pairs are available for use. Exemplary haptens comprise, but are not limited to, biotin, des-biotin and other derivatives, dinitrophenol, dansyl, fluorescein, Cy5, and digoxigenin. For biotin, a capture agent may be avidin, streptavidin, or antibodies. Antibodies may be used as capture agents for the other haptens (many dye-antibody pairs being commercially available, e.g., Molecular Probes, Eugene, Oreg.).

In some aspects, the analysis and/or sequence determination can be carried out at room temperature for best preservation of tissue morphology with low background noise and error reduction. In some embodiments, the analysis and/or sequence determination comprises eliminating error accumulation as sequencing proceeds.

In some embodiments, the analysis and/or sequence determination involves washing to remove unbound polynucleotides, thereafter revealing a fluorescent product for imaging.

In some aspects, the detecting involves using detection methods such as flow cytometry; sequencing; probe binding and electrochemical detection; pH alteration; catalysis induced by enzymes bound to DNA tags; quantum entanglement; Raman spectroscopy; terahertz wave technology; and/or scanning electron microscopy. In some aspects, the flow cytometry is mass cytometry or fluorescence-activated flow cytometry. In some aspects, the detecting comprises performing microscopy, scanning mass spectrometry or other imaging techniques described herein. In such aspects, the detecting comprises determining a signal, e.g., a fluorescent signal.

In some aspects, the detection (comprising imaging) is carried out using any of a number of different types of microscopy, e.g., confocal microscopy, two-photon microscopy, light-field microscopy, intact tissue expansion microscopy, and/or CLARITY™-optimized light sheet microscopy (COLM).

In some embodiments, fluorescence microscopy is used for detection and imaging of the detection probe. In some aspects, a fluorescence microscope is an optical microscope that uses fluorescence and phosphorescence instead of, or in addition to, reflection and absorption to study properties of organic or inorganic substances. In fluorescence microscopy, a sample is illuminated with light of a wavelength which excites fluorescence in the sample. The fluoresced light, which is usually at a longer wavelength than the illumination, is then imaged through a microscope objective. Two filters may be used in this technique; an illumination (or excitation) filter which ensures the illumination is near monochromatic and at the correct wavelength, and a second emission (or barrier) filter which ensures none of the excitation light source reaches the detector. Alternatively, these functions may both be accomplished by a single dichroic filter. The “fluorescence microscope” comprises any microscope that uses fluorescence to generate an image, whether it is a more simple set up like an epifluorescence microscope, or a more complicated design such as a confocal microscope, which uses optical sectioning to get better resolution of the fluorescent image.

In some embodiments, confocal microscopy is used for detection and imaging of the detection probe. Confocal microscopy uses point illumination and a pinhole in an optically conjugate plane in front of the detector to eliminate out-of-focus signal. As only light produced by fluorescence very close to the focal plane can be detected, the image's optical resolution, particularly in the sample depth direction, is much better than that of wide-field microscopes. However, as much of the light from sample fluorescence is blocked at the pinhole, this increased resolution is at the cost of decreased signal intensity, so long exposures are often required. As only one point in the sample is illuminated at a time, 2D or 3D imaging requires scanning over a regular raster (e.g., a rectangular pattern of parallel scanning lines) in the specimen. The achievable thickness of the focal plane is defined mostly by the wavelength of the used light divided by the numerical aperture of the objective lens, but also by the optical properties of the specimen. The thin optical sectioning possible makes these types of microscopes particularly good at 3D imaging and surface profiling of samples. CLARITY™-optimized light sheet microscopy (COLM) provides an alternative microscopy for fast 3D imaging of large clarified samples. COLM interrogates large immunostained tissues, permits increased speed of acquisition and results in a higher quality of generated data.

Other types of microscopy that can be employed comprise bright field microscopy, oblique illumination microscopy, dark field microscopy, phase contrast, differential interference contrast (DIC) microscopy, interference reflection microscopy (also known as reflected interference contrast, or RIC), single plane illumination microscopy (SPIM), super-resolution microscopy, laser microscopy, electron microscopy (EM), Transmission electron microscopy (TEM), Scanning electron microscopy (SEM), reflection electron microscopy (REM), Scanning transmission electron microscopy (STEM) and low-voltage electron microscopy (LVEM), scanning probe microscopy (SPM), atomic force microscopy (ATM), ballistic electron emission microscopy (BEEM), chemical force microscopy (CFM), conductive atomic force microscopy (C-AFM), electrochemical scanning tunneling microscope (ECSTM), electrostatic force microscopy (EFM), fluidic force microscope (FluidFM), force modulation microscopy (FMM), feature-oriented scanning probe microscopy (FOSPM), kelvin probe force microscopy (KPFM), magnetic force microscopy (MFM), magnetic resonance force microscopy (MRFM), near-field scanning optical microscopy (NSOM) (or SNOM, scanning near-field optical microscopy, SNOM, Piezoresponse Force Microscopy (PFM), PSTM, photon scanning tunneling microscopy (PSTM), PTMS, photothermal microspectroscopy/microscopy (PTMS), SCM, scanning capacitance microscopy (SCM), SECM, scanning electrochemical microscopy (SECM), SGM, scanning gate microscopy (SGM), SHPM, scanning Hall probe microscopy (SHPM), SICM, scanning ion-conductance microscopy (SICM), SPSM spin polarized scanning tunneling microscopy (SPSM), SSRM, scanning spreading resistance microscopy (SSRM), SThM, scanning thermal microscopy (SThM), STM, scanning tunneling microscopy (STM), STP, scanning tunneling potentiometry (STP), SVM, scanning voltage microscopy (SVM), and synchrotron x-ray scanning tunneling microscopy (SXSTM), and intact tissue expansion microscopy (exM).

In some embodiments, sequencing can be performed in situ. In situ sequencing typically involves incorporation of a labeled nucleotide (e.g., fluorescently labeled mononucleotides or dinucleotides) in a sequential, template-dependent manner or hybridization of a labeled primer (e.g., a labeled random hexamer) to a nucleic acid template such that the identities (e.g., nucleotide sequence) of the incorporated nucleotides or labeled primer extension products can be determined, and consequently, the nucleotide sequence of the corresponding template nucleic acid. Aspects of in situ sequencing are described, for example, in Mitra et al., Anal Biochem 2003; 320:55-65, and Lee et al., Science 2014; 343(6177):1360-63. In addition, examples of methods and systems for performing in situ sequencing are described in U.S. 2016/0024555, U.S. 2019/0194709, and in U.S. Pat. Nos. 10,138,509, 10,494,662 and 10,179,932, all of which are herein incorporated by reference in their entireties. Exemplary techniques for in situ sequencing comprise, but are not limited to, STARmap (described for example in Wang et al., Science 2018; 361(6499):5691), MERFISH (described for example in Moffitt and Zhuang, Methods in Enzymology 2016; 572:1-49), hybridization-based in situ sequencing (HybISS) (described for example in Gyllborg et al., Nucleic Acids Res 2020; 48(19):e112, and FISSEQ (described for example in U.S. 2019/0032121).

In some embodiments, sequencing can be performed by sequencing-by-synthesis (SBS). In some embodiments, a sequencing primer is complementary to sequences at or near the one or more barcode(s). In such embodiments, sequencing-by-synthesis can comprise reverse transcription and/or amplification in order to generate a template sequence from which a primer sequence can bind. Exemplary SBS methods comprise those described for example, but not limited to, U.S. 2007/0166705, U.S. 2006/0188901, U.S. Pat. No. 7,057,026, U.S. 2006/0240439, U.S. 2006/0281109, U.S. 2011/005986, U.S. 2005/0100900, U.S. Pat. No. 9,217,178, U.S. 2009/0118128, U.S. 2012/0270305, U.S. 2013/0260372, and U.S. 2013/0079232, all of which are herein incorporated by reference in their entireties.

In some embodiments, sequencing can be performed by sequential fluorescence hybridization (e.g., sequencing by hybridization). Sequential fluorescence hybridization can involve sequential hybridization of detection probes comprising an oligonucleotide and a detectable label.

In some embodiments, sequencing can be performed using single molecule sequencing by ligation. Such techniques utilize DNA ligase to incorporate oligonucleotides and identify the incorporation of such oligonucleotides. The oligonucleotides typically have different labels that are correlated with the identity of a particular nucleotide in a sequence to which the oligonucleotides hybridize. Aspects and features involved in sequencing by ligation are described, for example, in Shendure et al., Science 2005; 309:1728-32, and in U.S. Pat. Nos. 5,599,675; 5,750,341; 6,969,488; 6,172,218; and 6,306,597. all of which are herein incorporated by reference in their entireties.

In some embodiments, the barcodes of the probes are targeted by detectably labeled detection oligonucleotides, such as fluorescently labeled oligonucleotides. In some embodiments, one or more decoding schemes are used to decode the signals, such as fluorescence, for sequence determination. In any of the embodiments herein, barcodes (e.g., primary and/or secondary barcode sequences) can be analyzed (e.g., detected or sequenced) using any suitable methods or techniques, comprising those described herein, such as RNA sequential probing of targets (RNA SPOTs), sequential fluorescent in situ hybridization (seqFISH), single-molecule fluorescent in situ hybridization (smFISH), multiplexed error-robust fluorescence in situ hybridization (MERFISH), hybridization-based in situ sequencing (HybISS), in situ sequencing, targeted in situ sequencing, fluorescent in situ sequencing (FISSEQ), or spatially-resolved transcript amplicon readout mapping (STARmap). In some embodiments, the methods provided herein comprise analyzing the barcodes by sequential hybridization and detection with a plurality of labelled probes (e.g., detection oligonucleotides). Exemplary decoding schemes are described in Eng et al., Nature 2019; 568(7751):235-239; Chen et al., Science 2015; 348(6233):aaa6090; Gyllborg et al., Nucleic Acids Res 2020; 48(19):el12; U.S. Pat. No. 10,457,980; U.S. 2016/0369329, U.S. 2017/0220733; and WO 2018/026873, all of which are incorporated by reference in their entirety. In some embodiments, these assays enable signal amplification, combinatorial decoding, and error correction schemes at the same time.

In some embodiments, nucleic acid hybridization can be used for sequencing. These methods utilize labeled nucleic acid decoder probes that are complementary to at least a portion of a barcode sequence. Multiplex decoding can be performed with pools of many different probes with distinguishable labels. Non-limiting examples of nucleic acid hybridization sequencing are described for example in U.S. Pat. No. 8,460,865, and in Gunderson et al., Genome Res 2004; 14:870-877, all of which are herein incorporated by reference in their entireties.

In some embodiments, real-time monitoring of DNA polymerase activity can be used during sequencing. For example, nucleotide incorporations can be detected through fluorescence resonance energy transfer (FRET), as described for example in Levene et al., Science 2003; 299:682-6, Lundquist et al., Opt Lett 2008; 33:1026-1028, and Korlach et al., PNAS 2008; 105, 1176-81, all of which are herein incorporated by reference in their entireties.

### V. Kits

Also provided herein are kits, for example comprising one or more polynucleotides, e.g., any of the probes and/or primers described in Section III, and reagents for performing the methods provided herein, for example reagents required for one or more steps comprising hybridization, ligation, amplification, detection, sequencing, and/or sample preparation as described herein. In some embodiments, the kit further comprises a labeling agent e.g., any described in Section VI(B)(ii). In some embodiments, the kit comprises one or more reagents for synchronizing polymerase activity described in Section II, e.g., a proteinase, an inhibitor of polymerase, a polymerase trap, and/or a limiting amount of dNTPs. In some embodiments, any or all of the polynucleotides are DNA molecules. In some embodiments, the target nucleic acid is a messenger RNA molecule. In some embodiments, the kit further comprises a ligase, for instance for forming a circular probe from the circularizable probe, e.g., a padlock probe. In some embodiments, the ligase has DNA-splinted DNA ligase activity. In some embodiments, the ligase has RNA-splinted ligase activity. In some embodiments, the kit further comprises a polymerase, for instance for performing amplification of the circularizable probe, e.g., using any of the methods described in Section II. In some embodiments, the kit further comprises a primer for amplification.

In some embodiments, disclosed herein is a kit for analyzing a biological sample, comprising: (i) a binding mixture comprising a plurality of complexes each comprising a polymerase bound to a primer, and a chelating agent, wherein the binding mixture is substantially free of deoxynucleoside triphosphates (dNTPs); (ii) a primer extension reaction mixture comprising dNTPs and a di-cation, wherein the primer extension reaction mixture is substantially free of the polymerase, and (iii) individual reagents for altering polymerase activity, including a proteinase, a temperature-sensitive polymer, a trap molecule, and an inhibitor of the trap molecule (e.g., described in Section II). In some embodiments, the primers in the plurality of complexes are the same. In some embodiments, the primers in two or more of the plurality of complexes are different. In some embodiments, the polymerase is Phi29 DNA polymerase. In some embodiments, the di-cation is Mg²⁺, Co²⁺, and/or Mn²⁺. In some embodiments, the temperature-sensitive polymer is temperature-sensitive heparin and/or a derivative thereof and/or a gel-forming molecule. In some instances, the trap molecule is heparin and/or a derivative thereof. In some cases, the inhibitor of the trap molecule is heparin lyase and/or a combination of lithium chloride and ethanol.

The various components of the kit may be present in separate containers or certain compatible components may be pre-combined into a single container. In some embodiments, the kits further contain instructions for using the components of the kit to practice the provided methods.

In some embodiments, the kits can contain reagents and/or consumables required for performing one or more steps of the provided methods. In some embodiments, the kits contain reagents for fixing, embedding, and/or permeabilizing the biological sample. In some embodiments, the kits contain reagents, such as enzymes and buffers for ligation and/or amplification, such as ligases and/or polymerases. In some aspects, the kit can also comprise any of the reagents described herein, e.g., a proteinase, a temperature-sensitive polymer, a trap molecule, and an inhibitor of the trap molecule. In some embodiments, the kits contain reagents for detection and/or sequencing, such as barcode detection probes or detectable labels. In some embodiments, the kits optionally contain other components, for example nucleic acid primers, enzymes and reagents, buffers, nucleotides, modified nucleotides, reagents for additional assays.

### VI. Biological Samples and Analytes

A. Samples

A sample disclosed herein can be or derived from any biological sample. Methods and compositions disclosed herein may be used for analyzing a biological sample, which may be obtained from a subject using any of a variety of techniques including, but not limited to, biopsy, surgery, and laser capture microscopy (LCM), and generally includes cells and/or other biological material from the subject. In addition to the subjects described above, a biological sample can be obtained from a prokaryote such as a bacterium, an archaea, a virus, or a viroid. A biological sample can also be obtained from non-mammalian organisms (e.g., a plant, an insect, an arachnid, a nematode, a fungus, or an amphibian). A biological sample can also be obtained from a eukaryote, such as a tissue sample, a patient derived organoid (PDO) or patient derived xenograft (PDX). A biological sample from an organism may comprise one or more other organisms or components therefrom. For example, a mammalian tissue section may comprise a prion, a viroid, a virus, a bacterium, a fungus, or components from other organisms, in addition to mammalian cells and non-cellular tissue components. Subjects from which biological samples can be obtained can be healthy or asymptomatic individuals, individuals that have or are suspected of having a disease (e.g., a patient with a disease such as cancer) or a pre-disposition to a disease, and/or individuals in need of therapy or suspected of needing therapy.

The biological sample can include any number of macromolecules, for example, cellular macromolecules and organelles (e.g., mitochondria and nuclei). The biological sample can include nucleic acids (such as DNA or RNA), proteins/polypeptides, carbohydrates, and/or lipids. The biological sample can be obtained as a tissue sample, such as a tissue section, biopsy, a core biopsy, needle aspirate, or fine needle aspirate. The sample can be a fluid sample, such as a blood sample, urine sample, or saliva sample. The sample can be a skin sample, a colon sample, a cheek swab, a histology sample, a histopathology sample, a plasma or serum sample, a tumor sample, living cells, cultured cells, a clinical sample such as, for example, whole blood or blood-derived products, blood cells, or cultured tissues or cells, including cell suspensions. In some embodiments, the biological sample may comprise cells which are deposited on a surface.

Biological samples can be derived from a homogeneous culture or population of the subjects or organisms mentioned herein or alternatively from a collection of several different organisms, for example, in a community or ecosystem.

Biological samples can include one or more diseased cells. A diseased cell can have altered metabolic properties, gene expression, protein expression, and/or morphologic features. Examples of diseases include inflammatory disorders, metabolic disorders, nervous system disorders, and cancer. Cancer cells can be derived from solid tumors, hematological malignancies, cell lines, or obtained as circulating tumor cells. Biological samples can also include fetal cells and immune cells.

Biological samples can include analytes (e.g., protein, RNA, and/or DNA) embedded in a 3D matrix. In some embodiments, amplicons (e.g., rolling circle amplification products) derived from or associated with analytes (e.g., protein, RNA, and/or DNA) can be embedded in a 3D matrix. In some embodiments, a 3D matrix may comprise a network of natural molecules and/or synthetic molecules that are chemically and/or enzymatically linked, e.g., by crosslinking. In some embodiments, a 3D matrix may comprise a synthetic polymer. In some embodiments, a 3D matrix comprises a hydrogel.

In some embodiments, a substrate herein can be any support that is insoluble in aqueous liquid and which allows for positioning of biological samples, analytes, features, and/or reagents (e.g., probes) on the support. In some embodiments, a biological sample can be attached to a substrate. Attachment of the biological sample can be irreversible or reversible, depending upon the nature of the sample and subsequent steps in the analytical method. In certain embodiments, the sample can be attached to the substrate reversibly by applying a suitable polymer coating to the substrate, and contacting the sample to the polymer coating. The sample can then be detached from the substrate, e.g., using an organic solvent that at least partially dissolves the polymer coating. Hydrogels are examples of polymers that are suitable for this purpose.

In some embodiments, the substrate can be coated or functionalized with one or more substances to facilitate attachment of the sample to the substrate. Suitable substances that can be used to coat or functionalize the substrate include, but are not limited to, lectins, poly-lysine, antibodies, and polysaccharides.

A variety of steps can be performed to prepare or process a biological sample for and/or during an assay. Except where indicated otherwise, the preparative or processing steps described below can generally be combined in any manner and in any order to appropriately prepare or process a particular sample for and/or analysis.

### (i) Tissue Sectioning

A biological sample can be harvested from a subject (e.g., via surgical biopsy, whole subject sectioning) or grown in vitro on a growth substrate or culture dish as a population of cells, and prepared for analysis as a tissue slice or tissue section. Grown samples may be sufficiently thin for analysis without further processing steps. Alternatively, grown samples, and samples obtained via biopsy or sectioning, can be prepared as thin tissue sections using a mechanical cutting apparatus such as a vibrating blade microtome. As another alternative, in some embodiments, a thin tissue section can be prepared by applying a touch imprint of a biological sample to a suitable substrate material.

The thickness of the tissue section can be a fraction of (e.g., less than 0.9, 0.8, 0.7, 0.6, 0.5, 0.4, 0.3, 0.2, or 0.1) the maximum cross-sectional dimension of a cell. However, tissue sections having a thickness that is larger than the maximum cross-section cell dimension can also be used. For example, cryostat sections can be used, which can be, e.g., 10-20 μm thick.

More generally, the thickness of a tissue section typically depends on the method used to prepare the section and the physical characteristics of the tissue, and therefore sections having a wide variety of different thicknesses can be prepared and used. For example, the thickness of the tissue section can be at least 0.1, 0.2, 0.3, 0.4, 0.5, 0.7, 1.0, 1.5, 2, 3, 4, 5, 6, 7, 8, 9, 10, 12, 13, 14, 15, 20, 30, 40, or 50 μm. Thicker sections can also be used if desired or convenient, e.g., at least 70, 80, 90, or 100 μm or more. Typically, the thickness of a tissue section is between 1-100 μm, 1-50 μm, 1-30 μm, 1-25 μm, 1-20 μm, 1-15 μm, 1-10 μm, 2-8 μm, 3-7 μm, or 4-6 μm, but as mentioned above, sections with thicknesses larger or smaller than these ranges can also be analysed.

Multiple sections can also be obtained from a single biological sample. For example, multiple tissue sections can be obtained from a surgical biopsy sample by performing serial sectioning of the biopsy sample using a sectioning blade. Spatial information among the serial sections can be preserved in this manner, and the sections can be analysed successively to obtain three-dimensional information about the biological sample.

### (ii) Freezing

In some embodiments, the biological sample (e.g., a tissue section as described above) can be prepared by deep freezing at a temperature suitable to maintain or preserve the integrity (e.g., the physical characteristics) of the tissue structure. The frozen tissue sample can be sectioned, e.g., thinly sliced, onto a substrate surface using any number of suitable methods. For example, a tissue sample can be prepared using a chilled microtome (e.g., a cryostat) set at a temperature suitable to maintain both the structural integrity of the tissue sample and the chemical properties of the nucleic acids in the sample. Such a temperature can be, e.g., less than −15° C., less than −20° C., or less than −25° C.

(iii) Fixation and Postfixation

In some embodiments, the biological sample can be prepared using formalin-fixation and paraffin-embedding (FFPE), which are established methods. In some embodiments, cell suspensions and other non-tissue samples can be prepared using formalin-fixation and paraffin-embedding. Following fixation of the sample and embedding in a paraffin or resin block, the sample can be sectioned as described above. Prior to analysis, the paraffin-embedding material can be removed from the tissue section (e.g., deparaffinization) by incubating the tissue section in an appropriate solvent (e.g., xylene) followed by a rinse (e.g., 99.5% ethanol for 2 minutes, 96% ethanol for 2 minutes, and 70% ethanol for 2 minutes).

As an alternative to formalin fixation described above, a biological sample can be fixed in any of a variety of other fixatives to preserve the biological structure of the sample prior to analysis. For example, a sample can be fixed via immersion in ethanol, methanol, acetone, paraformaldehyde (PFA)-Triton, and combinations thereof.

In some embodiments, acetone fixation is used with fresh frozen samples, which can include, but are not limited to, cortex tissue, mouse olfactory bulb, human brain tumor, human post-mortem brain, and breast cancer samples. When acetone fixation is performed, pre-permeabilization steps (described below) may not be performed. Alternatively, acetone fixation can be performed in conjunction with permeabilization steps.

In some embodiments, the methods provided herein comprises one or more post-fixing (also referred to as postfixation) steps. In some embodiments, one or more post-fixing step is performed after contacting a sample with a polynucleotide disclosed herein, e.g., one or more probes such as a circular or circularizable probe. In some embodiments, one or more post-fixing step is performed after a hybridization complex comprising a probe and a target is formed in a sample. In some embodiments, one or more post-fixing step is performed prior to a ligation reaction disclosed herein, such as the ligation to circularize a probe.

In some embodiments, one or more post-fixing step is performed after contacting a sample with a binding or labeling agent (e.g., an antibody or antigen binding fragment thereof) for a non-nucleic acid analyte such as a protein analyte. The labeling agent can comprise a nucleic acid molecule (e.g., reporter oligonucleotide) comprising a sequence corresponding to the labeling agent and therefore corresponds to (e.g., uniquely identifies) the analyte. In some embodiments, the labeling agent can comprise a reporter oligonucleotide comprising one or more barcode sequences.

A post-fixing step may be performed using any suitable fixation reagent disclosed herein, for example, 3% (w/v) paraformaldehyde in DEPC-PBS.

### (iv) Embedding

As an alternative to paraffin embedding described above, a biological sample can be embedded in any of a variety of other embedding materials to provide structural substrate to the sample prior to sectioning and other handling steps. In some cases, the embedding material can be removed e.g., prior to analysis of tissue sections obtained from the sample. Suitable embedding materials include, but are not limited to, waxes, resins (e.g., methacrylate resins), epoxies, and agar.

In some embodiments, the biological sample can be embedded in a matrix (e.g., a hydrogel matrix). Embedding the sample in this manner typically involves contacting the biological sample with a hydrogel such that the biological sample becomes surrounded by the hydrogel. For example, the sample can be embedded by contacting the sample with a suitable polymer material, and activating the polymer material to form a hydrogel. In some embodiments, the hydrogel is formed such that the hydrogel is internalized within the biological sample.

In some embodiments, the biological sample is immobilized in the hydrogel via cross-linking of the polymer material that forms the hydrogel. Cross-linking can be performed chemically and/or photochemically, or alternatively by any other suitable hydrogel-formation method.

The composition and application of the hydrogel-matrix to a biological sample typically depends on the nature and preparation of the biological sample (e.g., sectioned, non-sectioned, type of fixation). As one example, where the biological sample is a tissue section, the hydrogel-matrix can include a monomer solution and an ammonium persulfate (APS) initiator/tetramethylethylenediamine (TEMED) accelerator solution. As another example, where the biological sample consists of cells (e.g., cultured cells or cells disassociated from a tissue sample), the cells can be incubated with the monomer solution and APS/TEMED solutions. For cells, hydrogel-matrix gels are formed in compartments, including but not limited to devices used to culture, maintain, or transport the cells. For example, hydrogel-matrices can be formed with monomer solution plus APS/TEMED added to the compartment to a depth ranging from about 0.1 μm to about 2 mm.

Additional methods and aspects of hydrogel embedding of biological samples are described for example in Chen et al., Science 347(6221):543-548, 2015, the entire contents of which is incorporated herein by reference.

### (v) Staining and Immunohistochemistry (IHC)

To facilitate visualization, biological samples can be stained using a wide variety of stains and staining techniques. In some embodiments, for example, a sample can be stained using any number of stains and/or immunohistochemical reagents. One or more staining steps may be performed to prepare or process a biological sample for an assay described herein or may be performed during and/or after an assay. In some embodiments, the sample can be contacted with one or more nucleic acid stains, membrane stains (e.g., cellular or nuclear membrane), cytological stains, or combinations thereof. In some examples, the stain may be specific to proteins, phospholipids, DNA (e.g., dsDNA, ssDNA), RNA, an organelle or compartment of the cell. The sample may be contacted with one or more labeled antibodies (e.g., a primary antibody specific for the analyte of interest and a labeled secondary antibody specific for the primary antibody). In some embodiments, cells in the sample can be segmented using one or more images taken of the stained sample.

In some embodiments, the stain is performed using a lipophilic dye. In some examples, the staining is performed with a lipophilic carbocyanine or aminostyryl dye, or analogs thereof (e.g, DiI, DiO, DiR, DiD). Other cell membrane stains may include FM and RH dyes or immunohistochemical reagents specific for cell membrane proteins. In some examples, the stain may include but is not limited to, acridine orange, acid fuchsin, Bismarck brown, carmine, coomassie blue, cresyl violet, DAPI, eosin, ethidium bromide, acid fuchsine, haematoxylin, Hoechst stains, iodine, methyl green, methylene blue, neutral red, Nile blue, Nile red, osmium tetroxide, ruthenium red, propidium iodide, rhodamine, (e.g., rhodamine B), or safranine, or derivatives thereof. In some embodiments, the sample may be stained with haematoxylin and eosin (H&E). The sample can be stained using hematoxylin and eosin (H&E) staining techniques, using Papanicolaou staining techniques, Masson's trichrome staining techniques, silver staining techniques, Sudan staining techniques, and/or using Periodic Acid Schiff (PAS) staining techniques. PAS staining is typically performed after formalin or acetone fixation. In some embodiments, the sample can be stained using Romanowsky stain, including Wright's stain, Jenner's stain, Can-Grunwald stain, Leishman stain, and Giemsa stain.

In some embodiments, biological samples can be destained. Any suitable methods of destaining or discoloring a biological sample may be utilized, and generally depend on the nature of the stain(s) applied to the sample. For example, in some embodiments, one or more immunofluorescent stains are applied to the sample via antibody coupling. Such stains can be removed using techniques such as cleavage of disulfide linkages via treatment with a reducing agent and detergent washing, chaotropic salt treatment, treatment with antigen retrieval solution, and treatment with an acidic glycine buffer. Methods for multiplexed staining and destaining are described, for example, in Bolognesi et al., J. Histochem. Cytochem. 2017; 65(8): 431-444, Lin et al., Nat Commun. 2015; 6:8390, Pirici et al., J. Histochem. Cytochem. 2009; 57:567-75, and Glass et al., J. Histochem. Cytochem. 2009; 57:899-905, the entire contents of each of which are incorporated herein by reference.

### (vi) Isometric Expansion

In some embodiments, a biological sample embedded in a matrix (e.g., a hydrogel) can be isometrically expanded. Isometric expansion methods that can be used include hydration, a preparative step in expansion microscopy, as described in, e.g., Chen et al., Science 347(6221):543-548, 2015 and U.S. Pat. No. 10,059,990, which are herein incorporated by reference in their entireties.

Isometric expansion can be performed by anchoring one or more components of a biological sample to a gel, followed by gel formation, proteolysis, and swelling. In some embodiments, analytes in the sample, products of the analytes, and/or probes associated with analytes in the sample can be anchored to the matrix (e.g., hydrogel). Isometric expansion of the biological sample can occur prior to immobilization of the biological sample on a substrate, or after the biological sample is immobilized to a substrate. In some embodiments, the isometrically expanded biological sample can be removed from the substrate prior to contacting the substrate with probes disclosed herein.

In general, the steps used to perform isometric expansion of the biological sample can depend on the characteristics of the sample (e.g., thickness of tissue section, fixation, cross-linking), and/or the analyte of interest (e.g., different conditions to anchor RNA, DNA, and protein to a gel).

In some embodiments, proteins in the biological sample are anchored to a swellable gel such as a polyelectrolyte gel. sDNA and/or RNA in a biological sample can also be anchored to the swellable gel via a suitable linker. Examples of such linkers include, but are not limited to, 6-((Acryloyl)amino) hexanoic acid (Acryloyl-X SE) (available from ThermoFisher, Waltham, MA), Label-IT Amine (available from MirusBio, Madison, WI) and Label X (described for example in Chen et al., Nat. Methods 13:679-684, 2016, and U.S. Pat. No. 10,059,990, the entire contents of which are incorporated herein by reference).

Isometric expansion of the sample can increase the spatial resolution of the subsequent analysis of the sample. The increased resolution in spatial profiling can be determined by comparison of an isometrically expanded sample with a sample that has not been isometrically expanded.

In some embodiments, a biological sample is isometrically expanded to a size at least 2×, 2.1×, 2.2×, 2.3×, 2.4×, 2.5×, 2.6×, 2.7×, 2.8×, 2.9×, 3×, 3.1×, 3.2×, 3.3×, 3.4×, 3.5×, 3.6×, 3.7×, 3.8×, 3.9×, 4×, 4.1×, 4.2×, 4.3×, 4.4×, 4.5×, 4.6×, 4.7×, 4.8×, or 4.9× its non-expanded size. In some embodiments, the sample is isometrically expanded to at least 2× and less than 20× of its non-expanded size.

(vii) Crosslinking and De-Crosslinking

In some embodiments, the biological sample is reversibly cross-linked prior to or during an in situ assay. In some aspects, the analytes, polynucleotides and/or amplification product (e.g., amplicon) of an analyte or a probe bound thereto can be anchored to a polymer matrix. For example, the polymer matrix can be a hydrogel. In some embodiments, one or more of the polynucleotide probe(s) and/or amplification product (e.g., amplicon) thereof can be modified to contain functional groups that can be used as an anchoring site to attach the polynucleotide probes and/or amplification product to a polymer matrix. In some embodiments, a modified probe comprising oligo dT may be used to bind to mRNA molecules of interest, followed by reversible or irreversible crosslinking of the mRNA molecules.

In some embodiments, the biological sample is immobilized in a hydrogel via cross-linking of the polymer material that forms the hydrogel. Cross-linking can be performed chemically and/or photochemically, or alternatively by any other suitable hydrogel-formation method. A hydrogel may include a macromolecular polymer gel including a network. Within the network, some polymer chains can optionally be cross-linked, although cross-linking does not always occur.

In some embodiments, a hydrogel can include hydrogel subunits, such as, but not limited to, acrylamide, bis-acrylamide, polyacrylamide and derivatives thereof, poly(ethylene glycol) and derivatives thereof (e.g. PEG-acrylate (PEG-DA), PEG-RGD), gelatin-methacryloyl (GelMA), methacrylated hyaluronic acid (MeHA), polyaliphatic polyurethanes, polyether polyurethanes, polyester polyurethanes, polyethylene copolymers, polyamides, polyvinyl alcohols, polypropylene glycol, polytetramethylene oxide, polyvinyl pyrrolidone, polyacrylamide, poly(hydroxyethyl acrylate), and poly(hydroxyethyl methacrylate), collagen, hyaluronic acid, chitosan, dextran, agarose, gelatin, alginate, protein polymers, methylcellulose, and the like, and combinations thereof.

In some embodiments, a hydrogel includes a hybrid material, e.g., the hydrogel material includes elements of both synthetic and natural polymers. Examples of suitable hydrogels are described, for example, in U.S. Pat. Nos. 6,391,937, 9,512,422, and 9,889,422, and in U.S. Patent Application Publication Nos. 2017/0253918, 2018/0052081 and 2010/0055733, the entire contents of each of which are incorporated herein by reference.

In some embodiments, the hydrogel can form the substrate. In some embodiments, the substrate includes a hydrogel and one or more second materials. In some embodiments, the hydrogel is placed on top of one or more second materials. For example, the hydrogel can be pre-formed and then placed on top of, underneath, or in any other configuration with one or more second materials. In some embodiments, hydrogel formation occurs after contacting one or more second materials during formation of the substrate. Hydrogel formation can also occur within a structure (e.g., wells, ridges, projections, and/or markings) located on a substrate.

In some embodiments, hydrogel formation on a substrate occurs before, contemporaneously with, or after probes are provided to the sample. For example, hydrogel formation can be performed on the substrate already containing the probes.

In some embodiments, hydrogel formation occurs within a biological sample. In some embodiments, a biological sample (e.g., tissue section) is embedded in a hydrogel. In some embodiments, hydrogel subunits are infused into the biological sample, and polymerization of the hydrogel is initiated by an external or internal stimulus.

In embodiments in which a hydrogel is formed within a biological sample, functionalization chemistry can be used. In some embodiments, functionalization chemistry includes hydrogel-tissue chemistry (HTC). Any hydrogel-tissue backbone (e.g., synthetic or native) suitable for HTC can be used for anchoring biological macromolecules and modulating functionalization. Non-limiting examples of methods using HTC backbone variants include CLARITY, PACT, ExM, SWITCH and ePACT. In some embodiments, hydrogel formation within a biological sample is permanent. For example, biological macromolecules can permanently adhere to the hydrogel allowing multiple rounds of interrogation. In some embodiments, hydrogel formation within a biological sample is reversible.

In some embodiments, additional reagents are added to the hydrogel subunits before, contemporaneously with, and/or after polymerization. For example, additional reagents can include but are not limited to oligonucleotides (e.g., probes), endonucleases to fragment DNA, fragmentation buffer for DNA, DNA polymerase enzymes, dNTPs used to amplify the nucleic acid and to attach the barcode to the amplified fragments. Other enzymes can be used, including without limitation, RNA polymerase, ligase, proteinase K, and DNAse. Additional reagents can also include reverse transcriptase enzymes, including enzymes with terminal transferase activity, primers, and switch oligonucleotides. In some embodiments, optical labels are added to the hydrogel subunits before, contemporaneously with, and/or after polymerization.

In some embodiments, HTC reagents are added to the hydrogel before, contemporaneously with, and/or after polymerization. In some embodiments, a cell labeling agent is added to the hydrogel before, contemporaneously with, and/or after polymerization. In some embodiments, a cell-penetrating agent is added to the hydrogel before, contemporaneously with, and/or after polymerization.

Hydrogels embedded within biological samples can be cleared using any suitable method. For example, electrophoretic tissue clearing methods can be used to remove biological macromolecules from the hydrogel-embedded sample. In some embodiments, a hydrogel-embedded sample is stored before or after clearing of hydrogel, in a medium (e.g., a mounting medium, methylcellulose, or other semi-solid mediums).

In some embodiments, a method disclosed herein comprises de-crosslinking the reversibly cross-linked biological sample. The de-crosslinking does not need to be complete. In some embodiments, only a portion of crosslinked molecules in the reversibly cross-linked biological sample are de-crosslinked and allowed to migrate.

(viii) Tissue Permeabilization and Treatment

In some embodiments, a biological sample can be permeabilized to facilitate transfer of species (such as probes) into the sample. If a sample is not permeabilized sufficiently, the transfer of species (such as probes) into the sample may be too low to enable adequate analysis. Conversely, if the tissue sample is too permeable, the relative spatial relationship of the analytes within the tissue sample can be lost. Hence, a balance between permeabilizing the tissue sample enough to obtain good signal intensity while still maintaining the spatial resolution of the analyte distribution in the sample is desirable.

In general, a biological sample can be permeabilized by exposing the sample to one or more permeabilizing agents. Suitable agents for this purpose include, but are not limited to, organic solvents (e.g., acetone, ethanol, and methanol), cross-linking agents (e.g., paraformaldehyde), detergents (e.g., saponin, Triton X-100™ or Tween-20™), and enzymes (e.g., trypsin, proteases). In some embodiments, the biological sample can be incubated with a cellular permeabilizing agent to facilitate permeabilization of the sample. Additional methods for sample permeabilization are described, for example, in Jamur et al., Method Mol. Biol. 588:63-66, 2010, the entire contents of which are incorporated herein by reference. Any suitable method for sample permeabilization can generally be used in connection with the samples described herein.

In some embodiments, the biological sample can be permeabilized by adding one or more lysis reagents to the sample. Examples of suitable lysis agents include, but are not limited to, bioactive reagents such as lysis enzymes that are used for lysis of different cell types, e.g., gram positive or negative bacteria, plants, yeast, mammalian, such as lysozymes, achromopeptidase, lysostaphin, labiase, kitalase, lyticase, and a variety of other commercially available lysis enzymes.

Other lysis agents can additionally or alternatively be added to the biological sample to facilitate permeabilization. For example, surfactant-based lysis solutions can be used to lyse sample cells. Lysis solutions can include ionic surfactants such as, for example, sarcosyl and sodium dodecyl sulfate (SDS). More generally, chemical lysis agents can include, without limitation, organic solvents, chelating agents, detergents, surfactants, and chaotropic agents.

In some embodiments, the biological sample can be permeabilized by non-chemical permeabilization methods. For example, non-chemical permeabilization methods that can be used include, but are not limited to, physical lysis techniques such as electroporation, mechanical permeabilization methods (e.g., bead beating using a homogenizer and grinding balls to mechanically disrupt sample tissue structures), acoustic permeabilization (e.g., sonication), and thermal lysis techniques such as heating to induce thermal permeabilization of the sample.

Additional reagents can be added to a biological sample to perform various functions prior to analysis of the sample. In some embodiments, DNase and RNase inactivating agents or inhibitors such as proteinase K, and/or chelating agents such as EDTA, can be added to the sample. For example, a method disclosed herein may comprise a step for increasing accessibility of a nucleic acid for binding, e.g., a denaturation step to open up DNA in a cell for hybridization by a probe. For example, proteinase K treatment may be used to free up DNA with proteins bound thereto.

### (ix) Selective Enrichment of RNA Species

In some embodiments, where RNA is the analyte, one or more RNA analyte species of interest can be selectively enriched. For example, one or more species of RNA of interest can be selected by addition of one or more oligonucleotides to the sample. In some embodiments, the additional oligonucleotide is a sequence used for priming a reaction by an enzyme (e.g., a polymerase). For example, one or more primer sequences with sequence complementarity to one or more RNAs of interest can be used to amplify the one or more RNAs of interest, e.g., to generate cDNA, thereby selectively enriching these RNAs.

In some aspects, when two or more analytes are analyzed, a first and second probe that is specific for (e.g., specifically hybridizes to) each RNA or cDNA analyte can be used. For example, in some embodiments of the methods provided herein, templated ligation is used to detect gene expression in a biological sample. An analyte of interest (such as a protein), bound by a labeling agent or binding agent (e.g., an antibody or epitope binding fragment thereof), wherein the binding agent is conjugated or otherwise associated with a reporter oligonucleotide comprising a reporter sequence that identifies the binding agent, can be targeted for analysis. Probes may be hybridized to the reporter oligonucleotide and ligated in a templated ligation reaction to generate a product for analysis. In some embodiments, gaps between the probe oligonucleotides may first be filled prior to ligation, using, for example, Mu polymerase, DNA polymerase, RNA polymerase, reverse transcriptase, VENT polymerase, Taq polymerase, and/or any combinations, derivatives, and variants (e.g., engineered mutants) thereof. In some embodiments, the assay can further include extension or amplification of templated ligation products (e.g., by rolling circle amplification of a circular product generated in a templated ligation reaction).

A biological sample may comprise one or a plurality of analytes of interest. Methods for performing multiplexed assays to analyze two or more different analytes in a single biological sample are provided.

B. Analytes

The methods and compositions disclosed herein can be used to detect and analyze a wide variety of different analytes. In some aspects, an analyte can include any biological substance, structure, moiety, or component to be analyzed. In some aspects, a target disclosed herein may similarly include any analyte of interest. In some examples, a target or analyte can be directly or indirectly detected.

Analytes can be derived from a specific type of cell and/or a specific sub-cellular region. For example, analytes can be derived from cytosol, from cell nuclei, from mitochondria, from microsomes, and more generally, from any other compartment, organelle, or portion of a cell. Permeabilizing agents that specifically target certain cell compartments and organelles can be used to selectively release analytes from cells for analysis, and/or allow access of one or more reagents (e.g., probes for analyte detection) to the analytes in the cell or cell compartment or organelle.

The analyte may include any biomolecule or chemical compound, including a macromolecule such as a protein or peptide, a lipid or a nucleic acid molecule, or a small molecule, including organic or inorganic molecules. The analyte may be a cell or a microorganism, including a virus, or a fragment or product thereof. An analyte can be any substance or entity for which a specific binding partner (e.g. an affinity binding partner) can be developed. Such a specific binding partner may be a nucleic acid probe (for a nucleic acid analyte) and may lead directly to the generation of a RCA template (e.g. a padlock probe or other circularizable probe or probe set). Alternatively, the specific binding partner may be coupled to a nucleic acid, which may be detected using an RCA strategy, e.g. in an assay which uses or generates a circular nucleic acid molecule which can be the RCA template.

Analytes of particular interest may include nucleic acid molecules, such as DNA (e.g. genomic DNA, mitochondrial DNA, plastid DNA, viral DNA, etc.) and RNA (e.g. mRNA, microRNA, rRNA, snRNA, viral RNA, etc.), and synthetic and/or modified nucleic acid molecules, (e.g. including nucleic acid domains comprising or consisting of synthetic or modified nucleotides such as LNA, PNA, morpholino, etc.), proteinaceous molecules such as peptides, polypeptides, proteins or prions or any molecule which includes a protein or polypeptide component, etc., or fragments thereof, or a lipid or carbohydrate molecule, or any molecule which comprise a lipid or carbohydrate component. The analyte may be a single molecule or a complex that contains two or more molecular subunits, e.g. including but not limited to protein-DNA complexes, which may or may not be covalently bound to one another, and which may be the same or different. Thus in addition to cells or microorganisms, such a complex analyte may also be a protein complex or protein interaction. Such a complex or interaction may thus be a homo- or hetero-multimer. Aggregates of molecules, e.g. proteins may also be target analytes, for example aggregates of the same protein or different proteins. The analyte may also be a complex between proteins or peptides and nucleic acid molecules such as DNA or RNA, e.g. interactions between proteins and nucleic acids, e.g. regulatory factors, such as transcription factors, and DNA or RNA.

(i) Endogenous Analytes

In some embodiments, an analyte herein is endogenous to a biological sample and can include nucleic acid analytes and non-nucleic acid analytes. Methods and compositions disclosed herein can be used to analyze nucleic acid analytes (e.g., using a nucleic acid probe or probe set that directly or indirectly hybridizes to a nucleic acid analyte) and/or non-nucleic acid analytes (e.g., using a labeling agent that comprises a reporter oligonucleotide and binds directly or indirectly to a non-nucleic acid analyte) in any suitable combination.

Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral coat proteins, extracellular and intracellular proteins, antibodies, and antigen binding fragments. In some embodiments, the analyte is inside a cell or on a cell surface, such as a transmembrane analyte or one that is attached to the cell membrane. In some embodiments, the analyte can be an organelle (e.g., nuclei or mitochondria). In some embodiments, the analyte is an extracellular analyte, such as a secreted analyte. Exemplary analytes include, but are not limited to, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, an extracellular matrix protein, a posttranslational modification (e.g., phosphorylation, glycosylation, ubiquitination, nitrosylation, methylation, acetylation or lipidation) state of a cell surface protein, a gap junction, and an adherens junction.

Examples of nucleic acid analytes include DNA analytes such as single-stranded DNA (ssDNA), double-stranded DNA (dsDNA), genomic DNA, methylated DNA, specific methylated DNA sequences, fragmented DNA, mitochondrial DNA, in situ synthesized PCR products, and RNA/DNA hybrids. The DNA analyte can be a transcript of another nucleic acid molecule (e.g., DNA or RNA such as mRNA) present in a tissue sample.

Examples of nucleic acid analytes also include RNA analytes such as various types of coding and non-coding RNA. Examples of the different types of RNA analytes include messenger RNA (mRNA), including a nascent RNA, a pre-mRNA, a primary-transcript RNA, and a processed RNA, such as a capped mRNA (e.g., with a 5′ 7-methyl guanosine cap), a polyadenylated mRNA (poly-A tail at the 3′ end), and a spliced mRNA in which one or more introns have been removed. Also included in the analytes disclosed herein are non-capped mRNA, a non-polyadenylated mRNA, and a non-spliced mRNA. The RNA analyte can be a transcript of another nucleic acid molecule (e.g., DNA or RNA such as viral RNA) present in a tissue sample. Examples of a non-coding RNAs (ncRNA) that is not translated into a protein include transfer RNAs (tRNAs) and ribosomal RNAs (rRNAs), as well as small non-coding RNAs such as microRNA (miRNA), small interfering RNA (siRNA), Piwi-interacting RNA (piRNA), small nucleolar RNA (snoRNA), small nuclear RNA (snRNA), extracellular RNA (exRNA), small Cajal body-specific RNAs (scaRNAs), and the long ncRNAs such as Xist and HOTAIR. The RNA can be small (e.g., less than 200 nucleic acid bases in length) or large (e.g., RNA greater than 200 nucleic acid bases in length). Examples of small RNAs include 5.8S ribosomal RNA (rRNA), 5S rRNA, tRNA, miRNA, siRNA, snoRNAs, piRNA, tRNA-derived small RNA (tsRNA), and small rDNA-derived RNA (srRNA). The RNA can be double-stranded RNA or single-stranded RNA. The RNA can be circular RNA. The RNA can be a bacterial rRNA (e.g., 16s rRNA or 23s rRNA).

In some embodiments described herein, an analyte may be a denatured nucleic acid, wherein the resulting denatured nucleic acid is single-stranded. The nucleic acid may be denatured, for example, optionally using formamide, heat, or both formamide and heat. In some embodiments, the nucleic acid is not denatured for use in a method disclosed herein.

Methods and compositions disclosed herein can be used to analyze any number of analytes. For example, the number of analytes that are analyzed can be at least about 2, at least about 3, at least about 4, at least about 5, at least about 6, at least about 7, at least about 8, at least about 9, at least about 10, at least about 11, at least about 12, at least about 13, at least about 14, at least about 15, at least about 20, at least about 25, at least about 30, at least about 40, at least about 50, at least about 100, at least about 1,000, at least about 10,000, at least about 100,000 or more different analytes present in a region of the sample or within an individual feature of the substrate.

(ii) Labeling Agents

In some embodiments, provided herein are methods and compositions for analyzing endogenous analytes (e.g., RNA, ssDNA, cell surface or intracellular proteins and/or metabolites) in a sample using one or more labeling agents. In some embodiments, an analyte labeling agent may include an agent that interacts with an analyte (e.g., an endogenous analyte in a sample). In some embodiments, the labeling agents can comprise a reporter oligonucleotide that is indicative of the analyte or portion thereof interacting with the labelling agent. For example, the reporter oligonucleotide may comprise a barcode sequence that permits identification of the labeling agent. In some cases, the sample contacted by the labelling agent can be further contacted with a probe (e.g., a single-stranded probe sequence), that hybridizes to a reporter oligonucleotide of the labeling agent, in order to identify the analyte associated with the labeling agent. In some embodiments, the analyte labeling agent comprises an analyte binding moiety and a labeling agent barcode domain comprising one or more barcode sequences, e.g., a barcode sequence that corresponds to the analyte binding moiety and/or the analyte. An analyte binding moiety barcode includes to a barcode that is associated with or otherwise identifies the analyte binding moiety. In some embodiments, by identifying an analyte binding moiety by identifying its associated analyte binding moiety barcode, the analyte to which the analyte binding moiety binds can also be identified. An analyte binding moiety barcode can be a nucleic acid sequence of a given length and/or sequence that is associated with the analyte binding moiety. An analyte binding moiety barcode can generally include any of the variety of aspects of barcodes described herein.

In some embodiments, the method comprises one or more post-fixing (also referred to as post-fixation) steps after contacting the sample with one or more labeling agents.

In the methods and systems described herein, one or more labeling agents capable of binding to or otherwise coupling to one or more features may be used to characterize analytes, cells and/or cell features. In some instances, cell features include cell surface features. Analytes may include, but are not limited to, a protein, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, a gap junction, an adherens junction, or any combination thereof. In some instances, cell features may include intracellular analytes, such as proteins, protein modifications (e.g., phosphorylation status or other post-translational modifications), nuclear proteins, nuclear membrane proteins, or any combination thereof.

In some embodiments, an analyte binding moiety may include any molecule or moiety capable of binding to an analyte (e.g., a biological analyte, e.g., a macromolecular constituent). A labeling agent may include, but is not limited to, a protein, a peptide, an antibody (or an epitope binding fragment thereof), a lipophilic moiety (such as cholesterol), a cell surface receptor binding molecule, a receptor ligand, a small molecule, a bi-specific antibody, a bi-specific T-cell engager, a T-cell receptor engager, a B-cell receptor engager, a pro-body, an aptamer, a monobody, an affimer, a darpin, and a protein scaffold, or any combination thereof. The labeling agents can include (e.g., are attached to) a reporter oligonucleotide that is indicative of the cell surface feature to which the binding group binds. For example, the reporter oligonucleotide may comprise a barcode sequence that permits identification of the labeling agent. For example, a labeling agent that is specific to one type of cell feature (e.g., a first cell surface feature) may have coupled thereto a first reporter oligonucleotide, while a labeling agent that is specific to a different cell feature (e.g., a second cell surface feature) may have a different reporter oligonucleotide coupled thereto. For a description of exemplary labeling agents, reporter oligonucleotides, and methods of use, see, e.g., U.S. Pat. No. 10,550,429; U.S. Pat. Pub. 20190177800; and U.S. Pat. Pub. 20190367969, which are each incorporated by reference herein in their entirety.

In some embodiments, an analyte binding moiety includes one or more antibodies or antigen binding fragments thereof. The antibodies or antigen binding fragments including the analyte binding moiety can specifically bind to a target analyte. In some embodiments, the analyte is a protein (e.g., a protein on a surface of the biological sample (e.g., a cell) or an intracellular protein). In some embodiments, a plurality of analyte labeling agents comprising a plurality of analyte binding moieties bind a plurality of analytes present in a biological sample. In some embodiments, the plurality of analytes includes a single species of analyte (e.g., a single species of polypeptide). In some embodiments in which the plurality of analytes includes a single species of analyte, the analyte binding moieties of the plurality of analyte labeling agents are the same. In some embodiments in which the plurality of analytes includes a single species of analyte, the analyte binding moieties of the plurality of analyte labeling agents are the different (e.g., members of the plurality of analyte labeling agents can have two or more species of analyte binding moieties, wherein each of the two or more species of analyte binding moieties binds a single species of analyte, e.g., at different binding sites). In some embodiments, the plurality of analytes includes multiple different species of analyte (e.g., multiple different species of polypeptides).

In other instances, e.g., to facilitate sample multiplexing, a labeling agent that is specific to a particular cell feature may have a first plurality of the labeling agent (e.g., an antibody or lipophilic moiety) coupled to a first reporter oligonucleotide and a second plurality of the labeling agent coupled to a second reporter oligonucleotide.

In some aspects, these reporter oligonucleotides may comprise nucleic acid barcode sequences that permit identification of the labeling agent which the reporter oligonucleotide is coupled to. The selection of oligonucleotides as the reporter may provide advantages of being able to generate significant diversity in terms of sequence, while also being readily attachable to most biomolecules, e.g., antibodies, etc., as well as being readily detected, e.g., using the in situ detection techniques described herein.

Attachment (coupling) of the reporter oligonucleotides to the labeling agents may be achieved through any of a variety of direct or indirect, covalent or non-covalent associations or attachments. For example, oligonucleotides may be covalently attached to a portion of a labeling agent (such a protein, e.g., an antibody or antibody fragment) using chemical conjugation techniques (e.g., Lightning-Link® antibody labeling kits available from Innova Biosciences), as well as other non-covalent attachment mechanisms, e.g., using biotinylated antibodies and oligonucleotides (or beads that include one or more biotinylated linker, coupled to oligonucleotides) with an avidin or streptavidin linker. Antibody and oligonucleotide biotinylation techniques are available. See, e.g., Fang, et al., “Fluoride-Cleavable Biotinylation Phosphoramidite for 5′-end-Labeling and Affinity Purification of Synthetic Oligonucleotides,” Nucleic Acids Res. Jan. 15, 2003; 31(2):708-715, which is entirely incorporated herein by reference for all purposes. Likewise, protein and peptide biotinylation techniques have been developed and are readily available. See, e.g., U.S. Pat. No. 6,265,552, which is entirely incorporated herein by reference for all purposes. Furthermore, click reaction chemistry may be used to couple reporter oligonucleotides to labeling agents. Commercially available kits, such as those from Thunderlink and Abcam, and techniques common in the art may be used to couple reporter oligonucleotides to labeling agents as appropriate. In another example, a labeling agent is indirectly (e.g., via hybridization) coupled to a reporter oligonucleotide comprising a barcode sequence that identifies the label agent. For instance, the labeling agent may be directly coupled (e.g., covalently bound) to a hybridization oligonucleotide that comprises a sequence that hybridizes with a sequence of the reporter oligonucleotide. Hybridization of the hybridization oligonucleotide to the reporter oligonucleotide couples the labeling agent to the reporter oligonucleotide. In some embodiments, the reporter oligonucleotides are releasable from the labeling agent, such as upon application of a stimulus. For example, the reporter oligonucleotide may be attached to the labeling agent through a labile bond (e.g., chemically labile, photolabile, thermally labile, etc.) as generally described for releasing molecules from supports elsewhere herein.

In some cases, the labeling agent can comprise a reporter oligonucleotide and a label. A label can be fluorophore, a radioisotope, a molecule capable of a colorimetric reaction, a magnetic particle, or any other suitable molecule or compound capable of detection. The label can be conjugated to a labeling agent (or reporter oligonucleotide) either directly or indirectly (e.g., the label can be conjugated to a molecule that can bind to the labeling agent or reporter oligonucleotide). In some cases, a label is conjugated to a first oligonucleotide that is complementary (e.g., hybridizes) to a sequence of the reporter oligonucleotide.

In some embodiments, multiple different species of analytes (e.g., polypeptides) from the biological sample can be subsequently associated with the one or more physical properties of the biological sample. For example, the multiple different species of analytes can be associated with locations of the analytes in the biological sample. Such information (e.g., proteomic information when the analyte binding moiety(ies) recognizes a polypeptide(s)) can be used in association with other spatial information (e.g., genetic information from the biological sample, such as DNA sequence information, transcriptome information (e.g., sequences of transcripts), or both). For example, a cell surface protein of a cell can be associated with one or more physical properties of the cell (e.g., a shape, size, activity, or a type of the cell). The one or more physical properties can be characterized by imaging the cell. The cell can be bound by an analyte labeling agent comprising an analyte binding moiety that binds to the cell surface protein and an analyte binding moiety barcode that identifies that analyte binding moiety. Results of protein analysis in a sample (e.g., a tissue sample or a cell) can be associated with DNA and/or RNA analysis in the sample.

(iii) Products of Endogenous Analyte and/or Labeling Agent

In some embodiments, provided herein are methods and compositions for analyzing one or more products of an endogenous analyte and/or a labeling agent in a biological sample. In some embodiments, an endogenous analyte (e.g., a viral or cellular DNA or RNA) or a product (e.g., a hybridization product, a ligation product, an extension product (e.g., by a DNA or RNA polymerase), a replication product, a transcription/reverse transcription product, and/or an amplification product such as a rolling circle amplification (RCA) product) thereof is analyzed. In some embodiments, a labeling agent that directly or indirectly binds to an analyte in the biological sample is analyzed. In some embodiments, a product (e.g., a hybridization product, a ligation product, an extension product (e.g., by a DNA or RNA polymerase), a replication product, a transcription/reverse transcription product, and/or an amplification product such as a rolling circle amplification (RCA) product) of a labeling agent that directly or indirectly binds to an analyte in the biological sample is analyzed.

### VII. Applications

In some aspects, the provided embodiments can be applied in an in situ method of analyzing nucleic acid sequences, such as an in situ transcriptomic analysis or in situ sequencing, for example from intact tissues or samples in which the spatial information has been preserved. In some aspects, the embodiments can be applied in an imaging or detection method for multiplexed nucleic acid analysis. In some aspects, the provided embodiments can be used to identify or detect regions of interest in target nucleic acids.

In some aspects, the embodiments can be applied in investigative and/or diagnostic applications, for example, for characterization or assessment of particular cell or a tissue from a subject. Applications of the provided method can comprise biomedical research and clinical diagnostics. For example, in biomedical research, applications comprise, but are not limited to, spatially resolved gene expression analysis for biological investigation or drug screening. In clinical diagnostics, applications comprise, but are not limited to, detecting gene markers such as disease, immune responses, bacterial or viral DNA/RNA for patient samples.

In some aspects, the embodiments can be applied to visualize the distribution of genetically encoded markers in whole tissue at subcellular resolution, for example, chromosomal abnormalities (inversions, duplications, translocations, etc.), loss of genetic heterozygosity, the presence of gene alleles indicative of a predisposition towards disease or good health, likelihood of responsiveness to therapy, or in personalized medicine or ancestry.

### VIII. Terminology

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

The terms “polynucleotide,” “polynucleotide,” and “nucleic acid molecule,” used interchangeably herein, refer to polymeric forms of nucleotides of any length, either ribonucleotides or deoxyribonucleotides. Thus, this term comprises, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. The backbone of the polynucleotide can comprise sugars and phosphate groups (as may typically be found in RNA or DNA), or modified or substituted sugar or phosphate groups.

“Hybridization” as used herein may refer to the process in which two single-stranded polynucleotides bind non-covalently to form a stable double-stranded polynucleotide. In one aspect, the resulting double-stranded polynucleotide can be a “hybrid” or “duplex.” “Hybridization conditions” typically include salt concentrations of approximately less than 1 M, often less than about 500 mM and may be less than about 200 mM. A “hybridization buffer” includes a buffered salt solution such as 5% SSPE, or other such buffers. Hybridization temperatures can be as low as 5° C., but are typically greater than 22° C., and more typically greater than about 30° C., and typically in excess of 37° C. Hybridizations are often performed under stringent conditions, e.g., conditions under which a sequence will hybridize to its target sequence but will not hybridize to other, non-complementary sequences. Stringent conditions are sequence-dependent and are different in different circumstances. For example, longer fragments may require higher hybridization temperatures for specific hybridization than short fragments. As other factors may affect the stringency of hybridization, including base composition and length of the complementary strands, presence of organic solvents, and the extent of base mismatching, the combination of parameters is more important than the absolute measure of any one parameter alone. Generally stringent conditions are selected to be about 5° C. lower than the Tm for the specific sequence at a defined ionic strength and pH. The melting temperature Tm can be the temperature at which a population of double-stranded nucleic acid molecules becomes half dissociated into single strands. As indicated by standard references, a simple estimate of the Tm value may be calculated by the equation, Tm=81.5+0.41 (% G+C), when a nucleic acid is in aqueous solution at 1 M NaCl (see e.g., Anderson and Young, Quantitative Filter Hybridization, in Nucleic Acid Hybridization (1985), incorporated herein by reference in its entirety). Other references (e.g., Allawi and SantaLucia, Jr., Biochemistry, 36:10581-94 (1997), incorporated herein by reference in their entireties) include alternative methods of computation which take structural and environmental, as well as sequence characteristics into account for the calculation of Tm.

In general, the stability of a hybrid is a function of the ion concentration and temperature. Typically, a hybridization reaction is performed under conditions of lower stringency, followed by washes of varying, but higher, stringency. Exemplary stringent conditions include a salt concentration of at least 0.01 M to no more than 1 M sodium ion concentration (or other salt) at a pH of about 7.0 to about 8.3 and a temperature of at least 25° C. For example, conditions of 5×SSPE (750 mM NaCl, 50 mM sodium phosphate, 5 mM EDTA at pH 7.4) and a temperature of approximately 30° C. are suitable for allele-specific hybridizations, though a suitable temperature depends on the length and/or GC content of the region hybridized. In one aspect, “stringency of hybridization” in determining percentage mismatch can be as follows: 1) high stringency: 0.1×SSPE, 0.1% SDS, 65° C.; 2) medium stringency: 0.2×SSPE, 0.1% SDS, 50° C. (also referred to as moderate stringency); and 3) low stringency: 1.0×SSPE, 0.1% SDS, 50° C. It is understood that equivalent stringencies may be achieved using alternative buffers, salts and temperatures. For example, moderately stringent hybridization can refer to conditions that permit a nucleic acid molecule such as a probe to bind a complementary nucleic acid molecule. The hybridized nucleic acid molecules generally have at least 60% identity, including for example at least any of 70%, 75%, 80%, 85%, 90%, or 95% identity. Moderately stringent conditions can be conditions equivalent to hybridization in 50% formamide, 5×Denhardt's solution, 5×SSPE, 0.2% SDS at 42° C., followed by washing in 0.2×SSPE, 0.2% SDS, at 42° C. High stringency conditions can be provided, for example, by hybridization in 50% formamide, 5×Denhardt's solution, 5×SSPE, 0.2% SDS at 42° C., followed by washing in 0.1×SSPE, and 0.1% SDS at 65° C. Low stringency hybridization can refer to conditions equivalent to hybridization in 10% formamide, 5×Denhardt's solution, 6×SSPE, 0.2% SDS at 22° C., followed by washing in 1× SSPE, 0.2% SDS, at 37° C. Denhardt's solution contains 1% Ficoll, 1% polyvinylpyrolidone, and 1% bovine serum albumin (BSA). 20×SSPE (sodium chloride, sodium phosphate, ethylene diamide tetraacetic acid (EDTA)) contains 3M sodium chloride, 0.2M sodium phosphate, and 0.025 M EDTA. Other suitable moderate stringency and high stringency hybridization buffers and conditions are described, for example, in Sambrook et al., Molecular Cloning: A Laboratory Manual, 2nd ed., Cold Spring Harbor Press, Plainview, N.Y. (1989); and Ausubel et al., Short Protocols in Molecular Biology, 4th ed., John Wiley & Sons (1999).

Alternatively, substantial complementarity exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See M. Kanehisa, Nucleic Acids Res. 12:203 (1984), incorporated herein by reference in its entirety.

A “primer” used herein can be an oligonucleotide, either natural or synthetic, that is capable, upon forming a duplex with a polynucleotide template, of acting as a point of initiation of nucleic acid synthesis and being extended from its 3′ end along the template so that an extended duplex is formed. The sequence of nucleotides added during the extension process is determined by the sequence of the template polynucleotide. Primers usually are extended by a DNA polymerase.

“Ligation” may refer to the formation of a covalent bond or linkage between the termini of two or more nucleic acids, e.g., oligonucleotides and/or polynucleotides, in a template-driven reaction. The nature of the bond or linkage may vary widely and the ligation may be carried out enzymatically or chemically. As used herein, ligations are usually carried out enzymatically to form a phosphodiester linkage between a 5′ carbon terminal nucleotide of one oligonucleotide with a 3′ carbon of another nucleotide.

“Sequencing,” “sequence determination” and the like means determination of information relating to the nucleotide base sequence of a nucleic acid. Such information may include the identification or determination of partial as well as full sequence information of the nucleic acid. Sequence information may be determined with varying degrees of statistical reliability or confidence. In one aspect, the term includes the determination of the identity and ordering of a plurality of contiguous nucleotides in a nucleic acid. “High throughput digital sequencing” or “next generation sequencing” means sequence determination using methods that determine many (typically thousands to billions) of nucleic acid sequences in an intrinsically parallel manner, e.g., where DNA templates are prepared for sequencing not one at a time, but in a bulk process, and where many sequences are read out preferably in parallel, or alternatively using an ultra-high throughput serial process that itself may be parallelized. Such methods include but are not limited to pyrosequencing (for example, as commercialized by 454 Life Sciences, Inc., Branford, Conn.); sequencing by ligation (for example, as commercialized in the SOLiD™ technology, Life Technologies, Inc., Carlsbad, Calif.); sequencing by synthesis using modified nucleotides (such as commercialized in TruSeq™ and HiSeq™ technology by Illumina, Inc., San Diego, Calif; HeliScope™ by Helicos Biosciences Corporation, Cambridge, Ma.; and PacBio RS by Pacific Biosciences of California, Inc., Menlo Park, Calif), sequencing by ion detection technologies (such as Ion Torrent™ technology, Life Technologies, Carlsbad, Calif); sequencing of DNA nanoballs (Complete Genomics, Inc., Mountain View, Calif.); nanopore-based sequencing technologies (for example, as developed by Oxford Nanopore Technologies, LTD, Oxford, UK), and like highly parallelized sequencing methods. “Multiplexing” or “multiplex assay” herein may refer to an assay or other analytical method in which the presence and/or amount of multiple targets, e.g., multiple nucleic acid target sequences, can be assayed simultaneously by using more than one probes, each of which has at least one different detection characteristic, e.g., fluorescence characteristic (for example excitation wavelength, emission wavelength, emission intensity, FWHM (full width at half maximum peak height), or fluorescence lifetime) or a unique nucleic acid or protein sequence characteristic.

As used herein, the term “fluorescent label” comprises a signaling moiety that conveys information through the fluorescent absorption and/or emission properties of one or more molecules. Exemplary fluorescent properties comprise fluorescence intensity, fluorescence lifetime, emission spectrum characteristics and energy transfer.

The term “about” as used herein refers to the usual error range for the respective value readily known to the skilled person in this technical field. Reference to “about” a value or parameter herein comprises (and describes) embodiments that are directed to that value or parameter per se.

As used herein, the singular forms “a,” “an,” and “the” comprise plural referents unless the context clearly dictates otherwise. For example, “a” or “an” means “at least one” or “one or more.”

Throughout the present disclosure, various aspects are presented in a range format. It should be understood that the description in range format is merely for convenience and brevity and should not be construed as an inflexible limitation on the scope of the present disclosure. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the present disclosure. The upper and lower limits of these smaller ranges may independently be comprised in the smaller ranges, and are also encompassed within the present disclosure, subject to any specifically excluded limit in the stated range. Where the stated range comprises one or both of the limits, ranges excluding either or both of those comprised limits are also comprised in the present disclosure. This applies regardless of the breadth of the range.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, use of a), b), etc., or i), ii), etc. does not by itself connote any priority, precedence, or order of steps in the claims. Similarly, the use of these terms in the specification does not by itself connote any required priority, precedence, or order.

### EXAMPLES

The following examples are included for illustrative purposes only and are not intended to limit the scope of the present disclosure.

### Example 1: Synchronizing In Situ Rolling Circle Amplification (RCA) Using Temperature

This example describes methods for improving in situ target detection and image analysis, which can be achieved by synchronizing the polymerase activity (e.g., polymerization initiation and/or termination) with different temperatures to improve control over in situ RCA reactions.

Tissue samples are prepared for in situ analysis of one or more target genes for gene expression. Circularizable probes are added to the sample and allowed to hybridize to target nucleic acids (e.g., RNA) in the sample. Following probe hybridization, the tissue sample is washed to remove unbound probes. For circularizable probe ligation, the sample is incubated at 37° C. for 1 hour with a ligase (e.g., T4 DNA ligase) for ligation of the circularizable probes to form circular probes. Before an amplification step and optionally before the ligation step, probe(s) that do not specifically hybridize to target nucleic acids in the sample can be disassociated from the target nucleic acids in the sample (e.g., using a stringent washing step). A reaction mixture containing dNTPs, Mg²⁺, and optionally Ca²⁺, along with Phi29 polymerase and primers for the circularized probes, can be added to each tissue sample at low temperatures (e.g., 4° C.) to limit or prevent the RCA reaction from happening. To initiate or ramp up the RCA reaction, temperatures are elevated to 37° C. In this example, experimental and control samples are investigated as shown in Table E1 below.

TABLE E1
Samples for investigating temperature control
over polymerase activity

| Sample | Incubation | Reaction Conditions |
|---|---|---|
| Control | 4º C. for 60 minutes | 37° C. for 120 minutes |
| A | 4º C. for 60 minutes | 37° C. for 90 minutes, |
| | | 4º C. for 60 minutes, |
| | | 37° C. for 30 minutes |

Prior to RCA reaction initiation, both samples are cooled to 4° C. Phi29 polymerase and primers for the circularized probes are added to the samples and incubated for 60 minutes. This incubation allows the Phi29-primer complexes to diffuse through the sample and “find” circularized probes hybridized to the target nucleic acids prior to initiation of the RCA reaction. Unbound complexes are removed by washing each sample. Following the low temperature incubation, the sample temperature is increased to 37° C. to initiate or ramp up the RCA reaction. For the experimental sample (Sample A), the biological sample is then cooled down to 4° C. to slow down or pause the RCA reaction. Optionally, the sample can be contacted with additional reaction mixture (e.g., containing dNTPs, Mg²⁺, and optionally Ca²⁺), Phi29 polymerase, and/or primer. After the incubation at 4° C. is complete, the temperature is increased to 37° C. to allow the RCA reaction to resume.

After completion of the RCA reaction for each sample, samples are washed and contacted with probes (e.g., detectably labeled probes, or intermediate probes such as L-shaped probes comprising overhang regions for hybridization of detectably labeled probes) and imaged to detect signals associated with the RCA products. The methods disclosed herein are expected to result in greater uniformity of RCA products.

### Example 2: Synchronizing In Situ RCA Using Polymerase Binder and/or Inhibitor

To further assess the impact of synchronizing polymerase activity on amplification and the resulting RCA product homogeneity, another set of experiments is performed, essentially as described above in Example 1 and using a temperature-sensitive polymer. Polymeric compounds containing a heparin moiety and gel-forming molecules can be used to inhibit polymerase activity, including Phi29 polymerase activity. In this example, temperature-sensitive polymers (e.g., heparin and heparin-bearing poly(ε-caprolactone-co-lactide)-b-poly(ethylene glycol)-b-poly(ε-caprolactone-co-lactide); Hep-PCLA) are employed. In some particular examples, the polyanionic structure of heparin can mimic nucleic acids, making it useful for affinity binding to nucleic acid-binding proteins including DNA and RNA polymerases. As such, heparin can be used to trap molecules such as Phi29 polymerase, and heparin can bind to the nucleic acid binding site of a polymerase, thereby inhibiting the polymerase activity. In some particular examples, Hep-PCLA can be used, wherein at 25° C., Hep-PCLA is free flowing in aqueous solution and can be used to trap Phi29 polymerase and/or inhibit Phi29 polymerase activity. The gel window in which Hep-PCLA forms a gel covers 37° C., under with temperature Hep-PCLA polymerizes to form a hydrogel and may not trap Phi29 polymerase and/or inhibit Phi29 polymerase activity. Experimental and control groups are set up as shown in Table E2 below. Here, using heparin to trap molecules of a polymerase and optionally using a temperature sensitive polymer can afford added control over polymerase activity, wherein only after release from binding and/or inhibition (e.g., by heparin or Hep-PCLA) can a polymerase catalyze amplification, leading to synchronizing of polymerase activity and polymerization.

TABLE E2
Samples for investigating control over polymerase activity using
binder and/or inhibitor of the polymerase

| | | | Polymerase Binder |
|---|---|---|---|
| Sample | Incubation | Reaction Conditions | and/or Inhibitor |
| Control | 4º C. for 60 min | 37º C. for 120 minutes | None |
| A | 4º C. for 60 min | 25° C. for 120 minutes | Hep-PCLA |
| B | 4º C. for 60 min | 37º C. for 120 minutes | Hep-PCLA |

In this example, prior to RCA reaction initiation, all of the samples are cooled to 4° C. Phi29 polymerase, polymerase binder and/or inhibitor (e.g., heparin or a temperature-sensitive polymer such as Hep-PCLA), dNTPs, and primers for the circularized probes added to the samples and incubated for 60 minutes. This incubation allows the Phi29, polymerase binder and/or inhibitor, dNTPs, and primers to equilibrate prior to initiation of the RCA reaction. Unbound complexes are removed by washing each sample. Following the low temperature incubation, the sample temperature is increased to either 37° C. or 25° C. to initiate or ramp up the RCA reactions.

After completion of the RCA reaction for each sample, samples are washed and contacted with probes (e.g., detectably labeled probes, or intermediate probes such as L-shaped probes comprising overhang regions for hybridization of detectably labeled probes) and imaged to detect signals associated with the RCA products. The methods disclosed herein are expected to result in greater uniformity of RCA products.

### Example 3: Synchronizing In Situ RCA Using Reversible Polymerase Traps

To further assess the impact of synchronizing polymerase activity on amplification and the resulting RCA product homogeneity, another set of experiments is performed, essentially as described above in Example 1 and using a reversible polymerase trap. Polymeric compounds containing a heparin moiety and gel-forming molecules can be used to trap molecules of a polymerase and/or inhibit polymerase activity, including Phi29 polymerase activity. In this example, a heparin containing polymer is used to modulate the polymerization by Phi29 polymerase activity, wherein heparin lyase is further added to inactivate or degrade the polymerase inhibitor (e.g., heparin). Experimental and control groups are set up as shown in Table E3 below. Here, addition of heparin followed by heparin lyase (e.g., herparinase) can afford added control over polymerase activity, synchronizing polymerase activity and RCA product generation.

TABLE E3
Samples for investigating control over polymerase activity
using a reversible trap

| Sample | Incubation | Reaction Conditions | Reversible Trap |
|---|---|---|---|
| Control | 4º C. for 60 min | 37º C. for 120 minutes | None |
| A | 4º C. for 60 min | 37° C. for 120 minutes | Heparin and |
| | | | Heparin Lyase |

In this example, prior to RCA reaction initiation, both samples are cooled to 4° C. Phi29 polymerase, dNTPs, and primers for the circularized probes are added to the samples and incubated for 60 minutes. In Sample A, a reversible trapping molecule (e.g., heparin) is also added for the low temperature incubation. This incubation allows the Phi29, reversible trapping molecule, dNTPs, and primers to equilibrate prior to initiation of the RCA reaction. Unbound complexes are removed by washing each sample. Following the low temperature incubation, the sample temperature is increased to 37° C. to initiate or ramp up the RCA reactions. For Sample A, heparin inhibition is reversed by adding heparin lyase to the sample, which rapidly degrades heparin.

After completion of the RCA reaction for each sample, samples are washed and contacted with probes (e.g., detectably labeled probes, or intermediate probes such as L-shaped probes comprising overhang regions for hybridization of detectably labeled probes) and imaged to detect signals associated with the RCA products. The methods disclosed herein are expected to result in greater uniformity of RCA products.

### Example 4: Synchronizing In Situ RCA Using Polymerase Digestion

To further assess the impact of synchronizing polymerase activity on amplification and the resulting RCA product homogeneity, another set of experiments is performed, essentially as described above in Example 1 and using proteinase for polymerase digestion. Proteinases, protease, and peptidases are enzymes that cleave peptide bonds in proteins to produce smaller peptide fragments. Introduction of a proteinase (e.g., proteinase K) to a biological sample containing an active polymerase (e.g., Phi29 polymerase) results in polymerase digestion and inactivation. After heat inactivation of the proteinase K in the biological sample, additional polymerase can be added to the biological sample in preparation for one or more subsequent rounds of amplification to increase RCA product signal. Experimental and control groups are set up as shown in Table E4 below. Here, pairing the temperature modulations described in Example 1 with the addition of proteinase K can afford added polymerase activity control, wherein a lower temperature synchronizes initiation of polymerization by Phi29 and acute addition of proteinase K to inactivate Phi29 synchronizes termination of polymerization.

TABLE E4
Samples for investigating control over polymerase activity
using proteinase K

| Sample | Incubation | Reaction Conditions | Digestion |
|---|---|---|---|
| Control | 4º C. for 60 min | 37º C. for 90 minutes, | None |
| | | 4º C. for 60 minutes, | |
| | | 37° C. for 30 minutes | |
| A | 4º C. for 60 min | 37º C. for 90 minutes, | Proteinase K |
| | | 37° C. for 5 minutes | |
| | | (Proteinase K incubation), | |
| | | 90° C. for 5, | |
| | | 4° C. for 60 minutes, | |
| | | 37º C. for 30 minutes | |

In this example, prior to RCA reaction initiation, both samples are cooled to 4° C. Phi29 polymerase, dNTPs, and primers for the circularized probes are added to the samples and incubated for 60 minutes. This incubation allows the Phi29, dNTPs, and primers to equilibrate prior to initiation of the RCA reaction. Unbound complexes are removed by washing each sample. Following the low temperature incubation, the sample temperature is increased to 37° C. to initiate or ramp up the RCA reactions. In Sample A, a proteinase (e.g., proteinase K) is added at the conclusion of the first RCA reaction and incubated for 5 minutes to pause polymerization. The temperature of Sample A is then raised to 90° C. to inactivate proteinase K in the sample. Both samples are then cooled down to 4° C. in preparation for a second round of RCA. The samples are contacted with more Phi29 polymerase, and optionally, more reaction mixture (e.g., containing dNTPs and Mg²⁺) and/or primer. After the incubation at 4° C. is complete, the temperature is increased to 37° C. for a subsequent round of RCA reaction.

After completion of the RCA reaction for each sample, samples are washed and contacted with probes (e.g., detectably labeled probes, or intermediate probes such as L-shaped probes comprising overhang regions for hybridization of detectably labeled probes) and imaged to detect signals associated with the RCA products. The methods disclosed herein are expected to result in greater uniformity of RCA products.

### Example 5: Synchronizing In Situ RCA Using dNTP Limitation

To further assess the impact of synchronizing polymerase activity on amplification and the resulting RCA product homogeneity, another set of experiments is performed, essentially as described above in Example 1 and using deoxynucleoside triphosphate (dNTP) limitation. Nucleotide substrates are essential for the polymerization reaction. Therefore, providing a limiting amount of substrate (e.g., dNTPs and/or derivatives thereof) allows the polymerase (e.g., Phi29) to load substrate but stall during polymerization due to limited amount of substrate. The loading of polymerase and subsequent pause in polymerization can synchronize the reaction, which can resume upon addition of dNTPs and/or derivatives thereof that can be incorporated by the polymerase. Experimental and control groups are set up as shown in Table E5 below. Here, dNTP limitation can afford added polymerase activity control, by pausing polymerization and allowing the reaction to resume upon addition of more dNTPs.

TABLE E5
Samples for investigating control over polymerase activity using
dNTP limitation

| Sample | Incubation | Reaction Conditions | Initial [dNTP] |
|---|---|---|---|
| Control | 4º C. for 60 min | 37° C. for 120 | 500 μM dNTPs |
| | | minutes | (excess) |
| A | 4° C. for 60 min | 37º C. for 120 | 5 nM dNTPs |
| | | minutes | (limiting) |

In this example, the reaction mixture for the Control sample includes an excess amount of dNTPs (e.g., 500 μM), while the reaction mixture for Sample A includes a limiting amount of dNTPs (e.g., 5 nM). Prior to RCA reaction initiation, both samples are cooled to 4° C. Phi29 polymerase and primers for the circularized probes are added to the samples and incubated for 60 minutes. This incubation allows the Phi29, primers, and dNTPs to equilibrate prior to initiation of the RCA reaction. Unbound complexes are removed by washing each sample. Following the low temperature incubation, the sample temperature is increased to 37° C. to initiate or ramp up the RCA reactions. To Sample A, after raising the temperature to 37° C., an excess amount of dNTPs is added to allow the paused RCA reaction (due to the low temperature and the limiting amount of dNTPs) to resume.

After completion of the RCA reaction for each sample, samples are washed and contacted with probes (e.g., detectably labeled probes, or intermediate probes such as L-shaped probes comprising overhang regions for hybridization of detectably labeled probes) and imaged to detect signals associated with the RCA products. The methods disclosed herein are expected to result in greater uniformity of RCA products.

### Example 6: Synchronizing In Situ RCA Using Oscillating Temperature

To further assess the impact of temperature oscillation on the synchronization of RCA in situ, a set of experiments was performed in which RCA was conducted in sequential high-temperature and low-temperature stages. In situ analysis of gene expression was conducted using circularizable probes complementary to a plurality of target nucleic acids. In a first hybridization cycle following RCA, a set of probes for detecting RCPs were incubated with the sample and imaging was conducted. The probes were then stripped from the sample, and another set of probes were incubated with the sample for sequential hybridization with RCPs associated with one of the three genes, and imaging was conducted to detect the probes.

In these experiments, a sample of fresh frozen mouse brain tissue was incubated with a plurality of circularizable probes overnight at 50° C. to allow the probes to hybridize to transcripts of the panel of 23 RNAs. The sample was also contacted with a plurality of negative control probes. Following probe hybridization, the tissue sample was washed to remove unbound probes. To circularize the probes, a ligase was added to the sample and incubated with the sample to ligate the circularizable probes to form circular probes. Phi29 polymerase was added to each sample and incubated at 4° C. with the sample for 30 minutes (the first low-temperature incubation stage). The low temperature (4° C.) limits the RCA reaction during the incubation with Phi29 polymerase. Following the first low-temperature incubation stage, the sample temperature was then increased to 30° C. (the first high-temperature stage) for 5 minutes to ramp up RCA and the formation of RCPs in. Following the first high-temperature stage, the temperature was lowered to 4° C. for 30 minutes to limit RCA (the second low-temperature stage). Following the second low-temperature stage, the temperature was then increased to 30° C. for another 5 minutes (the second high-temperature stage), allowing RCA to resume. Following the second high-temperature stage, the temperature was lowered to 4° C. for 30 minutes (the third low-temperature stage). The temperature was then increased to 30° C. for another 5 minutes (the third high-temperature stage), allowing RCA to resume.

In the Control group, Phi29 polymerase was added to each sample and incubated at 30° C. for 2 hours.

After completion of the RCA reaction, the samples were washed, contacted with probes for hybridization with RCPs, and imaged to detect signals associated with the RCPs associated with the three genes. Imaging was performed at 40× magnification, and RCP object counts and RCP size was determined for RCPs. Subsequently, the detection probes were stripped from the sample, and the sample was contacted with additional probes for hybridization with RCPs associated with one of the genes. Imaging was then performed a second time at 40× magnification to image the RCPs, and RCP object counts and RCP size was determined.

Object counts were comparable between the oscillating temperature group and the control group, with the oscillating temperature group producing smaller RCPs compared to control group. The size distribution for RCPs produced in the oscillating temperature group was narrower than the size distribution for RCPs produced in the control group.

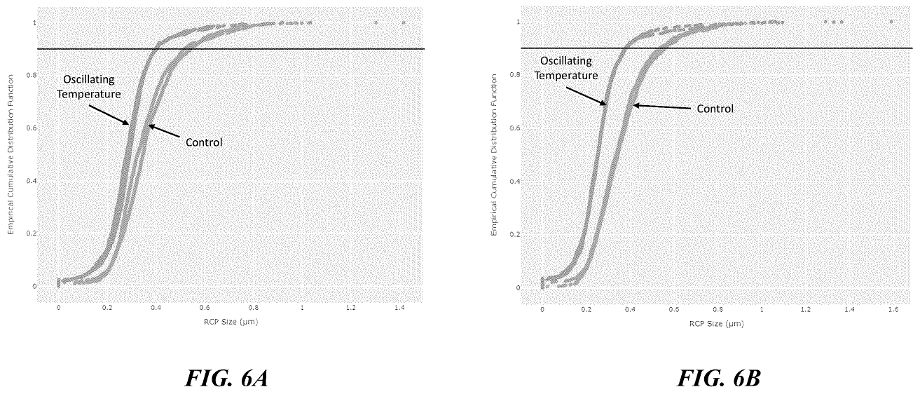

FIGS. 6A-6B show the cumulative distribution function of the size of RCPs produced during RCA performed according to the above oscillating temperature experimental protocol (“Oscillating Temperature”) compared to control. FIG. 6A shows a graph of the size of RCPs associated with the three target genes based on imaging of the sample. FIG. 6B shows a graph of the size of RCPs associated with one of the target genes based on imaging of the sample following the stripping and re-labelling of RCPs associated with one of the target genes. A trend of smaller RCP size was observed when RCA was conducted under oscillating temperatures. A narrower RCP size distribution and lower signal intensity was observed for samples at oscillating temperatures compared to control conditions. Taken together, these results indicate that temperature oscillation during RCA resulted in smaller RCPs and narrower size distribution of RCPs.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

1-74. (canceled)

75. A method for analyzing a biological sample, comprising:
a) contacting the biological sample with a polymerase under a first temperature,
wherein the biological sample comprises a plurality of circular nucleic acid molecules and the polymerase is capable of rolling circle amplification (RCA) of the plurality of circular nucleic acid molecules;
b) performing RCA in the biological sample using the polymerase under one or more second temperatures higher than the first temperature;
c) lowering the temperature of the biological sample to a third temperature;
d) performing RCA of the plurality of circular nucleic acid molecules using the polymerase under one or more fourth temperatures higher than the first temperature and/or the third temperature; and
e) detecting signals associated with RCA products of the plurality of circular nucleic acid molecules in the biological sample.

76. The method of claim 75, wherein the polymerase is substantially inactive under the first temperature.

77. The method of claim 75, wherein the biological sample and the polymerase are incubated under the first temperature for at least 5 minutes.

78. The method of claim 75, wherein the first temperature is lower than 10° C.

79. The method of claim 75, wherein the polymerase is more active under the one or more second temperatures than under the first temperature.

80. The method of claim 75, wherein the one or more second temperatures comprise a second temperature of at least 37° C.

81. The method of claim 75, wherein the RCA under the one or more second temperatures is performed in the same reaction mixture as that of the contacting step under the first temperature.

82. The method of claim 75, wherein the one or more second temperatures are between about 25° C. and about 60° C.

83. The method of claim 75, wherein the polymerase is substantially inactive under the third temperature.

84. The method of claim 75, wherein the third temperature is the same as the first temperature or no more 5° C. higher or lower than the first temperature.

85. The method of claim 75, wherein the third temperature is lower than 10° C.

86. The method of claim 75, further comprising an inactivation step between performing the RCA in b) and lowering the temperature in c) to inactivate and/or degrade the polymerase.

87. The method of claim 86, wherein the inactivation step comprises incubating the biological sample at a temperature that inactivates the polymerase.

88. The method of claim 86, wherein the inactivation step comprises treating the biological sample with a proteinase that degrades the polymerase.

89. The method of claim 86, further comprising contacting the biological sample with additional molecules of the polymerase after the inactivation step.

90. The method of claim 75, wherein the one or more fourth temperatures are the same as the one or more second temperatures.

91. The method of claim 75, wherein the biological sample is a tissue sample.

92. A method for analyzing a biological sample, comprising:
a) contacting the biological sample with a polymerase and a temperature-sensitive polymer under a first temperature, wherein:
the biological sample comprises a plurality of circular nucleic acid molecules, and
the temperature-sensitive polymer inhibits rolling circle amplification (RCA) by the polymerase under the first temperature;
b) performing RCA of the plurality of circular nucleic acid molecules using the polymerase under a second temperature, wherein the second temperature inactivates and/or degrades the temperature-sensitive polymer; and
c) detecting signals associated with RCA products of the plurality of circular nucleic acid molecules in the biological sample.

93. A method for analyzing a biological sample, comprising:
a) contacting the biological sample with a polymerase under a first temperature,
wherein the biological sample comprises a plurality of circular nucleic acid molecules and the polymerase is capable of rolling circle amplification (RCA) of the plurality of circular nucleic acid molecules;
b) performing RCA in the biological sample using the polymerase under one or more second temperatures higher than the first temperature;
c) contacting the biological sample with a proteinase that degrades the polymerase molecules in the biological sample;
d) inactivating the proteinase;
e) lowering the temperature of the biological sample to a third temperature;
f) contacting the biological sample with additional molecules of the polymerase to perform RCA in the biological sample;
g) performing RCA of the plurality of circular nucleic acid molecules using the polymerase under one or more fourth temperatures higher than the first temperature and/or the third temperature; and
h) detecting signals associated with RCA products of the plurality of circular nucleic acid molecules in the biological sample.

94. A method for analyzing a biological sample, comprising:
a) contacting the biological sample with a polymerase and a first amount of dNTPs comprising one or more bases,
wherein the biological sample comprises a plurality of circular nucleic acid molecules and the polymerase is capable of rolling circle amplification (RCA) of the plurality of circular nucleic acid molecules;
b) performing RCA in the biological sample using the polymerase until the first amount of dNTPs is exhausted;
c) contacting the biological sample with a second amount of dNTPs comprising all four of A, T, C, and G bases or variants or analogs thereof;
d) performing RCA in the biological sample using the polymerase and the second amount of dNTPs; and
e) detecting signals associated with RCA products of the plurality of circular nucleic acid molecules in the biological sample.
